(19) United States
(12) Patent Application Publication
(10) Pub. No.: US 2003/0103900 A1
(43) Pub. Date: Jun. 5, 2003

(54) Use of disease-associated organisms

(76) Inventor: Samir Chachoua, unknown (AU)

Correspondence Address: Ted W. Whitlock, 5323 SW 38th Avenue, Ft. Lauderdale, FL, 33312, US

(21) Appl. No.: 09/999,547

(22) Filed: Nov. 30, 2001

(51) Int. Cl. A61K 49/00; G06F 17/60; G06F 19/00; G01N 33/48; G01N 33/50

(52) U.S. Cl. 424/9.1; 702/19; 705/3

(57) ABSTRACT

A method for the identification, production and use of disease and condition specific diagnostic, therapeutic and preventative agents from naturally occurring microorganisms, organisms, extracts or modifications thereof, and from other chemical or physical agents. Diagnostic, screening and therapeutic devices are also disclosed.

### REFERENCE TO RELATED APPLICATIONS

This application claims the benefit of U.S. Provisional Application Serial No. 60/003,686, filed Sep. 15, 1995, now abandoned; U.S. patent application Ser. No. 08/713,984, filed Sep. 15, 1996, now abandoned; and U.S. patent application Ser. No. 09/800,089, filed Mar. 5, 2001.

### FIELD OF THE INVENTION

The present invention relates to methods for disease prevention, diagnosis, treatment, prognosis and monitoring. More particularly, the present invention relates to the identification and use of disease-associated organisms, elements and forces which may be used, whole or in part, in the diagnoses, therapy and prevention of a targeted disease or other unwanted bodily condition and/or facilitate a desired state.

### BACKGROUND OF THE INVENTION

A wide variety of chemically and physically based treatments for cancer and other tumorous conditions have been developed and are well known, including inter alia, various types of chemotherapy, photodynamic therapy, radiotherapy, immunotherapy and combinations of various treatments. Although it is desirable that such treatments be generally toxic only to cells of a targeted disease, it has been difficult to identify agents having such specificity. To the contrary, the use of known anti-tumor therapeutic agents such as chemotherapy and radiotherapy is typically designed with the hope that the agent being used is more toxic to cells of the targeted disease than to the normal cells of the patient's body. In fact, many such agents and therapies rely almost solely on an increased rate of cell division or other mitotic activity in cancer cells, which leads to increased absorption for overcoming the problem of general toxicity, and thereby enabling the subsequent toxic efficacy of the agents and techniques. Unfortunately, there are also normal cells in the body which have high mitotic activity, such as cells of the bone marrow and intestines, which often suffer from such therapeutic agents and therapies.

Indeed, many chemically based therapies for tumorous conditions involve the use of antimitotic drugs such as adriamycin, vincristine, cisplatin and methotrexate, all of which exhibit strong undesirable side-effects on the normal cells of the patient. Such adverse side effects include hair loss, extreme nausea, vomiting, fatigue, weakness, sterility, and damage to the kidneys and heart. Because such side effects can impact severely on the ability of a body's immune system to focus on the original pathogenic agents, and on the body's ability to rebound from the effects of both the targeted disease and the chemotherapy, there is a strong need for anticancer agents having greater specificity in their targeting capabilities. Conventional chemotherapeutic treatments, furthermore, are not often able to circumvent the protective barriers built by most cancers against a host immune system. Indeed, most cancers are able to develop resistance to specific chemotherapeutic agents before such agents are able to completely penetrate such protective barriers.

In addition to conventional therapies which target the destruction of diseased tissue through the absorption of toxic agents, more recently, the patient's own immune system has been stimulated to prevent the formation of micrometastases or eliminate metastases already formed. Such attempts have generally precipitated the formation by the patient's body of tumor specific T-killer cells by immunizing the patient with oncolysates, or lysates from tumor cells. With this approach, the problem arises that cell lysates tend not to be sufficiently immunogenic and therefore, fail to induce sufficient stimulation of the patient's immune system to effectively prevent the formation of metastases and that they may be stimulating a depleted response.

It is generally believed that tumor specific T-lymphocytes, when they are present, occur only at low frequency among the lymphocytes, and that the antigenicity and immunogenicity of tumor agents is generally weak. It is further believed that these tumor specific cells are usually not fully activated in tumor-bearing animals, needing, therefore, additional activation signals. Furthermore, autoimmune diseases are thought to be caused by microorganisms and their products/extracts so well camouflaged that the immune system has difficulty distinguishing the said microbes and their products/extracts from normal tissue components which such diseases attack. In a patient infected with AIDS, an infected T-cell will ordinarily be attacked only by another T-cell, which itself will become subsequently infected. Indeed, some physicians have attempted to overcome the body's tendency to continue providing only susceptible T-cells at the disease by using immunosuppressive agents to keep the body from being flooded with infected T-cells. It is not difficult even for those unskilled in the medical arts to recognize the conflicting logical errors inherent in such an approach.

A related problem associated with many known anti-disease therapies concerns the components typically used in vaccines made in accordance with the teachings of the prior art. With a few exceptions, vaccines for use in the prevention or treatment of a targeted disease are generally made from either the targeted disease itself, diseased tissue or other material which is similar to the targeted disease. For example, current AIDS vaccines tend to be made from fragments of the HN membrane or other relatively stable viral fragment. Unfortunately, there are several problems associated with such practice. First, such vaccines may serve merely to amplify an already ineffective or inappropriate immune response, or further deplete an immune response. AIDS vaccines may lead to the production of more T-cells which are susceptible to the HIV virus.

Another problem associated with such vaccines is the inherent risk of administering to the body pathogenic material which may have an activity toward the body beyond that which is known. Consequently, in addition to stimulating the body to increase its natural immune response to such a vaccine, there may be other consequences of vaccinating the body with such pathogenic material. Also, it is well known by medical practitioners that the disease strains from which many vaccines are made are actually grown on “continuous cell lines,” which, for the sake of economy, tend to be cancer-like tissue. It is not fully known whether such practice mayor may not cause the transfer into the patient's body of dynamic pathogenic material which may be “hidden” in the selected deactivated and “benign” disease fragments (e.g., a temporarily dormant element of genetic code).

Another such problem associated with the use of vaccines made from pathogenic material of or similar to the targeted disease, is that such use with a patient already infected by the targeted disease involves further taxing of an already weakened immune system. Such attempted stimulation of a weakened immune system by disease material may actually cause or aggravate what is known as “threshold inhibition phenomenon,” in which the immune system is unable to produce additional immunological agents due to its having already reached its maximum capacity for such production or due to immunologic inhibition by factors often related to the total bulk of disease manifestation. A still further problem with using vaccines made in accordance with teachings of the prior art, is that a targeted disease is often able to develop a resistance to such vaccines, just as it may develop a resistance toward components of the body's naturally stimulated immune system. One of the known defense mechanisms of many organisms which cause or stimulate disease is the capability of mutating with extreme rapidity in order to build such resistance.

With regard to solid tumor masses, it is generally believed that the dormant cellular center which typically comprises the central core of such masses, is protected by a growing barrier of diseased cells, making the dormant core, thereby, unsusceptible to current chemical absorption therapies their relative metabolic stasis and dormancy also places them in a state of resistance to radiotherapy. The mainstays of conventional therapy, radiation and chemotherapy exert maximal effects against rapidly dividing cells. Cancer cells not only are possessed of adaptive and mutational capacity which promotes their survival but also resist toxic agents by relative metabolic inactivity in their central core. The dormant cellular center may further provide for the production of tumor clones, which are resistant to toxic absorption therapies or radiation when the center is awakened following partial destruction of the tumor mass. It is readily apparent, therefore, that in addition to the need for anti-tumor treatments having greater specificity in their targeting capability is a need for activity not based on metabolic/physical/chemical toxicity.

One system which may overcome this situation involves the selective immunological targeting of the cells. This has proven difficult but in theory would not favor survival of the dormant disease cells. The problem seems amplified by the apparent ability of the outer cancer layers to repel most routine immune responses.

There is another system which can turn the therapeutic dilemma of dormant cells and immunologic repulsion/evasion by tumor masses into a therapeutic advantage. This involves the isolation and identification of specific organisms with specific cancer affinity such that they or extracts thereof maybe used to specifically attack or mark the cancer cells and mass (the process of marking or tagging involves the use of infections or other agents/factors/products/extracts thereof to mark a previously non-immunogenic tissue with immunologically reactive antigens. Infections may access the dormant cancer center with greater ease if anaerobic or microaerophilic viral infection may infect certain cancer cells with ease as many of the latter lack effective anti-viral responses. A large range of microorganisms may access cancer tissue due to its apparent resistance to immune function. The large number of infections with apparent affinity for cancer would suggest that infections may be found in cancer in its natural state. There have been numerous reports of this observation, ranging from Scott and Glover earlier this century to Livingston-Wheeler in the middle part of the century. Most of the early observers believed that the infections were the cause of the cancer; it is also known, however, that certain infections such as malaria, Newcastle's disease virus and even mumps (1974, Teruo Asada, Cancer 34:1907-1928) may bring about or extend a remission. Many infections are now said to be contaminants whereas some such as schistosomycetes (human bladder); hepatitis (human liver); human papilloma virus and cervical cancer are of recognized importance in the generation of human cancers; viruses are indisputable causative agents of many induced animal cancers.

Infections may therefore be implicated in the cause, the healing or seemingly exert no input on the overall clinical picture. Part of this mystery may be resolved by categorizing all infections that occur in cancer patients so that follow-up of cases can indicate if improvement or deterioration result.

There is also a need for treatments which are capable of inducing into action against an infectious or other agent a broad range of elements in the body's total immune system (e.g., neutrophils, eosinophils, basophils, etc.) which may not be as susceptible to the targeted infection, and which may have the ability to overcome any protective anti-immune barriers established by the targeted disease.

There is a long felt need for therapeutic methods which are effective in eliminating less massive, non-tumor pathogenic cellular materials, such as independent microorganisms contained in bodily fluids. Since chemical application is normally an effective way to contact such targeted cellular material, current therapies for such conditions are generally based on a chemical treatment which focuses on either producing cellular lysis of such pathogenic materials or inhibiting vital nutrient pathways or other processes vital to the life of the targeted disease or diseased material. Unfortunately, current chemotherapeutic treatments which attack diseased cells, also tend to affect non-targeted cells. An example of such treatment is the use of broad spectrum antibiotics which not only affect disease bacteria, but may affect the normal intestinal flora of “friendly” organisms, leading to further pathology such as diarrhea and other malabsorption phenomena.

Most chemically based therapies involve the use of purified non-living substances. Similar to that found in the treatment of tumorous diseases, a well known problem often associated with the treatment of non-tumor pathogenic materials with non-living chemically based therapies, is the tendency of the targeted disease to develop a resistance to the chemical agent. Current methods for manufacturing such chemical agents are both slow in response to the development of such chemical agent resistance, and are cost prohibitive for the specific targeting of a wide variety of diseases. One example of how the prior art deals with the rapidity with which such diseases can develop chemical resistance, is illustrated in the treatment of tuberculosis. Because the development of anti-tuberculosis agents using current methods is expensive, physicians often use a combination of therapeutic agents, hoping that the combined effect of such agents, each of which has become ineffective against the targeted disease when applied singly, will overcome such ineffectiveness. Similar logic has followed in general cancer therapy, where it is common practice to use a combination of chemotherapy with or without radiation. It is interesting to note, however, that on a broad scale such practice has not led to significant increased patient survival, which is indicative that such combination therapy of the prior art is effective in causing extension of survival in cancer patients.

Another example of how current methods for developing anti-disease therapeutic agents can impact society is the development of chemically based remedies targeting viruses. Unfortunately, the time and expense involved in the development of such remedies are too great for duplication across a wide spectrum of known diseases. As a result, drug companies tend to target only the most common diseases in their research and development efforts. Because each such effort may take years using current techniques of drug development, many diseases are allowed to run their course without the introduction of modem medical therapy. Indeed, the general public tends to believe that viruses (which are merely microorganisms too small to be observed through a light microscope) are not generally treatable by the use of modem medicine, only because such treatments have not been made available on a large scale. Such non-living chemically based treatments fail to incorporate the significant advantages attendant through the use of living organisms in the creation of such remedies and therapeutic systems.

Another problem associated with many known anti-disease therapies concerns the inability of such therapies to identify and target a true causative agent of the targeted disease. An example of this may be seen in the treatment of auto-immune diseases such as pemphigus. With pemphigus, there are painful sterile pustules, which are believed to be generated by an increased, altered, or overstimulated immunological response. Since no microorganism has been heretofore associated with the introduction of the pustules. Current anti-pemphigus therapy includes using cortisone for diminishing the increased immune activity. Although this therapy may be palliative and add comfort to a patient, it is by no means curative. A parallel may be drawn between this therapy and that which is commonly used in the treatment of cancer, wherein an unknown agent has stimulated an uncontrolled division of normal cells into cancerous material. Instead of applying steroids for reducing the diseased material, however, chemotherapy and radiotherapy are used, producing results similar to that of the treatment for pemphigus. Because neither therapy actually targets the disease causative agent(s), they are equally unsuccessful in their efficacy against the targeted disease, which efficacy may be only minimal at best.

A related dilemma commonly associated with known cancer therapies is a general inability to rid the body of the key cancer causing factor or factors, even when the cancerous-tissue is removed. Often times, a cancer therapy will include the surgical removal of various diseased cells, in hopes of removing the disease causative factor with such cells. Unfortunately, such surgical “detoxification” of the body is generally capable of possibly slowing down continuing cancer growth. There have been cases of leukemia recurrence following remission and bone marrow transplant where the leukemia cells bore more resemblance to the donor's cells than those of the patient. These were reported sporadically in the late 70's and early 80's in conversations with inventor and colleagues. This suggests a causative agent acting within the patient to convert normal cells to cancer cells as opposed to simple recurrence of the original cancer. The idea that cancer the disease may consist of more than just cancer the cells seemed to be borne out by the failure of leukophoresis to extend significantly patient survival time (leukophoresis is a process of selectively removing white blood cells (largely leukemic ones from the peripheral blood). If the leukemia disease process was largely represented by the leukemic cells then leukophoresis should have been expected to impact more significantly on the disease process. The concept of cancer spread being facilitated by loose cancer cells circulating was also challenged by the process of ascitic shunting.

Ascites is usually a terminal development in the progress of a cancer patient, representing the accumulation of several liters of peritoneal fluid around the cancer which has usually spread extensively in that cavity at the time. Such fluid contains variable quantities of free cancer cells and cancer fragments drainage of such fluid is often done weekly or as needed for patient comfort. Such a process is not only tiring but also cardiovascularly compromising. There is also repeated risk of hemorrhage perforation of some organ infection loculation of fluid etc. Ascitic shunts were designed to minimize patient discomfort by passing a catheter from the peritoneal cavity into the right atrium or major vein. There was a theoretic fear that pumping cancer-rich fluid would result in increased cancer seeding in areas such as the lungs and other organs that may not have been already involved. Cases reviewed by the inventor did not seem to manifest any such changes. There are no widespread reports of this anticipated consequence. It may be that such patients simply do not live long enough for such metastases to manifest but at one autopsy attended by inventor, no evidence of even micrometastases could be found in the lungs although ascitic fluid was rich in cellular content. It appeared that ascitic fluid may somehow alter cancer cells or that cancer cellular showers in the blood were not the only prerequisite for metastases.

Another problem associated with anti-disease therapies of the prior art concerns the diagnosis of various diseases. Because many diseases, especially cancers, have the ability to hide themselves from, or even to suppress a host immune system, such diseases are not detected until such time as prominent symptoms occur and the disease has become an immediate threat to the life of the host. One such disease is adenocarcinoma, or cancer of the ovaries, which has been referred to often by the general population as the “silent killer.” Unfortunately, the diagnosis of such diseases via conventional methods does not include the use of disease-associated agents which can detect the presence of, and therefore facilitate the diagnosis of, such diseases long before they become an immediate threat to human life.

Although a basic assumption in the medical arts is that every disease has at least a causative agent, no one method has been heretofore proposed for the ready identification of such causative agents across a wide spectrum of diseases. Still further, no one method has been proposed for developing effective treatments for the sundry diseases having unknown etiologies. In his research regarding the etiology of various diseases, the inventor has found that many fungal, bacterial and viral organisms which are normally treated as foreign matter by a healthy patient's immune system are often found unchallenged in the bodies of cancer patients. Others have investigated and reported on an apparent antagonistic relationship between certain infections and cancer. (See Microbiology and Cancer Therapy: A Review. H. Christine Reilly, 1953.; Nauts, H. et al., ACTA MED Scandinavia, 278, 145; 1-103, 1953.; Nauts, H. et al., Cancer Research 6, 205-216, 1946.; Beebe et al. JA.M.A. 49, 1495-1498, 1907.; Baroni, Arch Roumaines Path Expert et Micro, 11, 125-142, 1938.; Comsia, Compt Rend Soc de Biol, 99:900-901, 1928.; and Daels, Arch Hyg, 72:257-300, 1910).

In Pathogenesis of Cancer (The Freemont Foundation, 1955), Dr. John E. Gregory discloses findings concerning the potential relationship between cancer and unchallenged foreign microorganisms isolated in the bodies of diseased patients. Dr. Gregory teaches that most of the organisms are merely natural contaminants which may be disregarded as insignificant since, according to Dr. Gregory, there can be only one cancer virus and, thus, only one effective method of conquering a cancer. Since the work of Dr. Gregory, similar conclusions have been drawn by Dr. Virginia Livingston of the San Diego based Livingston-Wheeler Clinic. Dr. Livingston identified a microorganism, Progenitor cryptocides, which she believed is the main cause of cancer.

Although much knowledge has been acquired during the past century regarding the identification of various viral, bacterial and fungal organisms which are somehow related to various diseases, many questions have remained unanswered regarding the relationship between such organisms. The inability of researchers to either link directly organisms to the disease's etiology, or to link directly the various types of such organisms to each other (e.g., linking a certain virus to a certain bacteria, both of which have been identified in patients having the same or similar diseases) has resulted in great confusion regarding the potential use of such knowledge. Indeed, vaccines prepared and used against such organisms have often resulted in only limited success.

Studies have been conducted to determine whether anti-disease characteristics can be transferred into other organisms. A bacterial extract containing Clostridium hystolyticum was incubated with cancer tissue. (See Connell, Canadian M.A. J 33:363-370, 1935.). In 1947, the same organism was used in an attempt to mark cancer cells with bacterial antigens, which were then treated successfully with an antitoxin (see Parker et al., Proc Soc Exper Biol and Med, 66:461-467, 1947.). More recently, Volker Schirrmacher disclosed in U.S. Pat. No. 5,273,745 and German Patent No. 3806565, a similar technique in which inactivated autologous tumor cells are marked by incubating same with similarly inactivated Newcastle Disease Virus (NDV) in a serum-free medium. Because of the natural ability of the NDV to activate tumor specific T-cells, the number of such T-cells which are ultimately activated against the tumor subsequent to inoculation with the NDV vaccine created in accordance with the Schirrmacher teaching, is sufficiently greater than the number of cells the tumor itself is able to activate.

A particular problem which Parker et al. and Schirrmacher attempted to address was the capability of a cancerous malignancy to mask itself from the defenses of an immune system. Parker demonstrated that such a defensive mechanism might be overcome in some circumstances by using an antitoxin which can readily identify tagged cancer cells. Schirrmacher demonstrated that in certain circumstances a portion of the patient's immune system could also be stimulated to react to tagged cancer cells. However, neither the teaching of Parker et al., nor that of Schirrmacher has been replicated effectively in the ongoing war against cancer and other diseases of unknown etiology.

Regarding further Schirrmacher's teaching that a portion of the patient's immune system may be stimulated to react to the tagged cancer cells, such portion which the Schirrmacher vaccine is able to raise comprises less than one percent of the body's total known immune system. The Schirrmacher teaching fails to include eliciting a greater portion of a body's defense system against the targeted disease, or utilizing the benefits of disease-associated therapeutic agents, such as those made directly from a living microorganism for attacking the targeted disease. Furthermore, killed and tagged tumor cells which have been thus inactivated by virus and radiation, as taught by Schirrmacher, may bear only minimal resemblance to the living cancer cells inside a patient; and hence, can have only minimal overlap in resultant immunostimulation. Even if such minimal overlap results in an immune response which is greater than that stimulated by the presence of only the targeted disease, such overlap may still enable the targeted disease to mutate into a clone which is resistant to the anti-disease agents, thereby nullifying the overlapping response. The prior art practice of presenting the body with a vaccine constructed partially of the targeted disease and partially of other agents, without altering the living disease at the same time and without fortifying significantly the patient's immunological abilities, may result in dividing the body's immunological response against both the disease and the vaccine. This could be a more significant problem than that of minimal overlapping antigenicity.

### SUMMARY OF THE INVENTION

A primary objective of the present invention is to provide a method for creating disease- and condition-specific diagnostic, therapeutic and preventative agents from naturally occurring microorganisms, organism extracts or modifications thereof, and from other chemical or physical agents, which overcomes the aforementioned problems associated with therapeutic agents, therapies and methods for producing same in the prior art. Not only does the instant invention lead to maximum overlap between vaccine and living disease tissue, but some embodiments of the invention even cause the tagging of a vaccine directly to living disease tissue, for providing maximum specificity. Also, various embodiments of the therapeutic agents made via the method of the present invention have demonstrated an ability to stimulate an amplification of the host's known immunological functions, both specifically and non-specifically, and have even raised a hitherto unknown immunological therapeutic mechanism which relates to the red blood cells. The following three naturally occurring phenomena are indicative of the sources for the isolation of such disease- and condition-specific therapeutic agents: 1) Spontaneous remission; 2) Organ and Species resistance; and 3) Cellular redifferentiation.

The spontaneous remission of cancer and other incurable diseases in the form of miraculous healing has been described as a “healing crisis” ranging in duration from a few hours to several days, during which time dramatic symptoms of an acute infection (e.g., fever, chills and perspiration) were exhibited. Further, it is known that certain organs and systems of the body, such as the spleen, small intestine and muscular system, are rarely infected by metastatic diseases which readily encroach upon more susceptible bodily organs and systems, such as the lungs, liver and skeletal system. By practicing the method of the present invention, the disease resistance of these bodily organs and tissues as well as microorganisms isolated therefrom may be used in the production of disease-specific therapeutic agents for use in other, more susceptible areas of the body.

Certain non-microscopic, non-human, life forms are known to be resistant to the ravages of cancer and various other diseases. Great difficulty has been found in using anti-disease sera in humans, which has been extracted from animals. The limitation of this method lies in the nonspecificity of antisera developed from non-patient-specie life forms, especially with regard to the tendency of such sera to include high levels of an anti-patient-specie factor, which has often proven to be quite toxic. In practicing the method of the present invention, however, it has been discovered that various tumor specific therapeutic agents can be raised in vivo in different-specie life forms, subsequently “washed” and filtered for removing any anti-patient-specie factors. Indeed, such therapeutic agents have been used by the inventor in the treatment of humans without any attendant anti-human side effects.

Regarding cellular redifferentiation, there are reports of cases in which malignant tumors have suddenly redifferentiated into normal cells. Such reports often have noted the association of the redifferentiating tumors with some other abnormal occurrence within or about the body. For example, such redifferentiation has been reported in leukemia after the occurrence of a staphylococcal infection. Also, redifferentiation of cancer cells has been observed in tumor masses which were placed in proximity to a developing notochord, or grafted onto a salamander's regenerating stump. The association of such diverse occurrences with redifferentiation in various tumor types suggests that cancer cells are not irreversible, but rather, under certain conditions, can be returned to a normal state. (See Laclau, Compt Rend Soc de Biol 92:840-842, 1925; Nevorojkin, Vestnik Roentgenol Radiol 15: 344-345, 1935; Maisin, Compt Rend Soc de Biol 127: 1477-1478, 1938; Protti, Tumori 22:222-229, 1948.; Protti, Tumori 24: 14-24, 1950; Lewisohn, Science 94:7071, 1941; Lewisohn, Cancer Research 1 799-8066, 1941; and Suiguira, AAAS Approaches to Tumor Chemotherapy, 208-213, 1947).

The present invention provides a comprehensive method for identifying various disease-associated agents for use in treating a targeted disease. The agents include organisms, organism extracts and modifications thereof, chemical and mineral elements, and physical forces. The present invention further includes a method for producing various therapeutic agents from the disease-associated agents, and the use of such therapeutic agents in the prevention, diagnosis, treatment, prognosis and monitoring of a targeted disease or other unwanted bodily condition.

The present invention is particularly concerned with the treatment and prevention of diseases which compromise the body's ability to mount an effective immunological response. Such immunologically inhibiting diseases include, but are not limited to, Acquired Immune Deficiency Syndrome (“AIDS”), cancer, pathogenic angiogenesis and vascularization, systematic lupus erythromatosis, rheumatoid arthritis, inflammatory bowel disease, multiple sclerosis, Alzheimer's disease, muscular dystrophy, asthma, chronic fatigue syndrome, A.L.S., I.T.P., etc. Such illnesses which are treatable in accordance with the present invention include those that are caused by either an original infectious agent (e.g., Epstein Barr virus as it relates to Burkitt's lymphoma, Human Immunodeficiency Virus (“HIV”) as it is believed to relate to AIDS), or an opportunistic infectious agent attacking the body of a host which has been already weakened by an original infectious agent (e.g., pneumocystis pneumonia, cytomegalovirus, herpes, etc., as they relate to AIDS). More particularly, this invention relates to the identification and therapeutic use of various organisms, elements and forces which exhibit at least one of the following associative characteristics regarding a targeted disease: 1) a causative affinity toward a targeted disease; 2) a synergistic affinity toward a targeted disease; 3) a neutral affinity toward a targeted disease; 4) an infective affinity toward a targeted disease; 5) a regression-stimulating antagonism toward a targeted disease; or 6) a nemesis antagonism toward a targeted disease.

The present invention is also directed to the use of such organisms, elements and forces in the treatment and prevention of various naturally occurring conditions that are degenerative, for example, bodily conditions which are associated with “old age.” In each of these diseases or conditions, use of the disease-specific therapeutic agents resulting from the method of the present invention appears to exert a regenerative effect on the treated individual, a regulatory effect on the individual's immune system as well as exhibit the desired anti-disease activity. The therapies of the present invention have also demonstrated remarkably few side-effects, especially when compared with existing cancer and immunodeficient therapies, such as adjuvant chemotherapy, the high-dose application of lymphokines or the application of AZT. As such, the general purpose of the present invention is to provide a new and improved method for producing therapeutic agents for use in treating a targeted disease or condition, which method incorporates various disease-associated organisms, elements and forces in the creation of the therapeutic agents for overcoming the aforementioned problems associated with diagnostic, therapeutic and preventative agents and therapies of the prior art.

It is a further object of the present invention to provide a method for producing therapeutic agents which are effective for treating an illness arising from, or exacerbated by, the failure of a human's immune system to mount an adequate immune response to the illness.

It is still another object of the present invention to provide a method for producing therapeutic agents for a patient from various chemical or mineral agents, biological organisms, extracts or modifications thereof, which have been raised in a different specie than that of the patient.

It is still another object of the present invention to provide a method for producing therapeutic agents from various chemical or mineral agents, biological organisms, extracts or modifications thereof, which have been raised in vitro.

It is still further an object of the present invention to provide a method for producing therapeutic agents from various chemical or mineral agents, biological organisms, organism extracts or modifications thereof, which demonstrate antagonistic activity toward a targeted disease, the method including enhancing the antagonism of such agents or organisms toward cells of the targeted disease or diseased tissue such that the effectiveness of the therapeutic agent is increased.

It is still further an object of the present invention to provide a method for producing therapeutic agents from various chemical or mineral agents, biological organisms, organism extracts or modifications thereof, which demonstrate an affinity toward the targeted disease, the method including enhancing the affinity of such agents or organisms toward cells of the targeted disease or diseased tissue, such that the effectiveness of the therapeutic agent is increased.

It is still another object of the present invention to provide a method for producing a disease-specific antagonistic activity in cells with a selected chemical or mineral agent, or biological organism, extract or modification thereof, which agent or organism has not heretofore demonstrated antagonistic activity toward a targeted disease.

It is still another object of the present invention to provide a method for producing regenerative therapeutic agents from various chemical or mineral agents, biological organisms, extracts or modifications thereof, for use in therapies targeting the restoration of various tissues or bodily functions.

It is yet another object of the present invention to provide a method for treating a patient suffering from an illness which arises from, or is exacerbated by, the failure of his or her immune system to mount an adequate response to the illness, the method including the use of at least a therapeutic agent produced in accordance with the method of the present invention.

It is another object of the present invention to provide a method for treating a tumorous disease in a patient, the method including the use of at least a therapeutic agent created in accordance with the method of the present invention.

It is another object of the present invention to provide a method for treating naturally occurring degenerative conditions such as old age, the method including the use of at least a therapeutic agent produced in accordance with the method of the present invention.

It is yet another object of the present invention to provide a method for treating a patient suffering from an illness, the method including the application of various electromagnetic frequencies to the patient, which frequencies are associated with the targeted disease.

It is yet another object of the present invention to provide a method for treating a patient suffering from an illness, the method including the application of various electromagnetic frequencies to the patient, which frequencies are not associated with the targeted disease.

It is another object of the present invention to provide a method for producing beneficial changes to the immune responsiveness of patients having illnesses which compromise the ability of the body to mount an effective immunological response.

It is still another object of the present invention to provide a method for transferring from a microorganism to cells of the human body, various desirable characteristics of the microorganism.

It is still further an object of the present invention to provide a method for producing therapeutic agents for use in vaccinating, or otherwise treating, a patient in order to prevent the patient from acquiring a targeted disease, the therapeutic agent comprising a material which is neither the targeted disease, nor an extract or modification of the targeted disease.

Furthermore, during application of the method of the present invention, it has been discovered that red blood cells may be stimulated to play an active role in a patient's immunological defense system. While current genetic engineering efforts have concentrated generally on the modification of various cells having intricate pre-existing nuclear structure, the red blood cell has been heretofore overlooked by such efforts. Indeed, because the red blood cell has no nucleus and is normally in more abundant supply throughout the body than any other cell, it has been found that this cell is extremely useful as an ideal empty vessel for responding to, or expressing, desirable physical, chemical or genetic information. It is, therefore, another object of the present invention to provide a method for producing therapeutic agents from various chemical or mineral agents, biological organisms, organism extracts or modifications thereof, or physical forces, which therapeutic agents can illicit use of a patient's red blood cells for treating a targeted disease.

It is therefore another object of the present invention to provide a method for producing therapeutic agents from various chemical or mineral agents, physical forces, biological organisms, organism extracts or modifications thereof, which therapeutic agents can illicit use of a patient's red blood cells for inhibiting metastases, attacking cancer and other diseases. It is still further an object of the present invention to provide a method for diagnosing various diseases which have an ability to hide from, or suppress, a host immune system, the method including the use of at least a therapeutic agent created in accordance with the method of the present invention.

It is still further an object of the present invention to provide a system for creating therapeutic agents from various chemical or mineral agents, physical forces, biological organisms, extracts or modifications thereof, which system can be used to effectively anticipate the development of specific resistive changes in a targeted disease, such that therapeutic agents can be created for specifically and timely countering such anticipated resistive changes, thereby allowing for the economic creation and timely delivery of effective disease-specific therapeutic agents to the patient.

It is yet another object of the present invention to provide a method for applying therapeutic agents created in accordance with the method of the present invention that is economical to replicate and use.

These together with other objects of the present invention are pointed out with particularity in the claims herein and form part of this disclosure. The more important objects of the present invention have been outlined rather broadly in order that the detailed description thereof which follows may be better understood, and in order that the present contribution to the art may be better appreciated. For a better understanding of the present invention, its operational advantages and the specific objects attained by its uses, reference should be made to the ensuing descriptive matter and appended illustrations, in which there are disclosed various embodiments of the invention.

Those versed in the art will readily ascertain, however, that the present invention is capable of other embodiments and of being practiced and carried out in various other ways. In this respect, the various embodiments disclosed herein, and the arrangements of the various components of the instant invention set forth in the following description are for illustrative purposes, only, and are not intended to be limiting in scope. Those skilled in the art will appreciate, as well, that the conception upon which this disclosure is based, may be utilized readily as a basis for the designing of other methods and systems for carrying out the several purposes of the present invention. Said other methods may include, but are not limited to, those which include the substitution of other material sources as they become available, and which substantially perform the same function in substantially the same manner with substantially the same result as the various described components of the present invention. It is important, therefore, that the claims appended hereto be regarded as including such equivalent components, methods and systems insofar as these do not depart from the spirit and scope of the present invention.

### BRIEF DESCRIPTION OF THE DRAWINGS

FIGS. 1 and 2 illustrate two vials in which such in vitro reaction tests were conducted.

FIG. 2 shows a lymph node extracted from the same patient and cultured with Penicillium fungus; filaments surrounding the cancer began to develop within days. This organism demonstrated affinity to the cancer and could not be washed off the surface easily within days of co-culture, it could be recovered from the surface and from fragments deep within the mass.

As shown in FIG. 3, a Russel body marked by arrow, such bodies have often been noted in cancer biopsies. Early researchers thought them to indicate blastomycetes, inventor believes them to be yeast or fungal etiology.

FIG. 4 shows several coccoid forms forming a nest in a biopsy of breast cancer. In both FIGS. 3 and 4, note the total lack of any immune response.

FIG. 5 shows a sample of mixed coccal culture which was grown from such a “clean” blood culture of an AIDS patient.

FIG. 6 shows a culture of tubercule bacilli grown from a “clean” skin sample of a scleroderma patient.

FIG. 7 illustrates morphological changes, in which red central blood cells show extending proboscis.

FIG. 8 in which are shown spicules in the membrane of several red blood cells, two of which cells have also taken on a ring-like form with an apparent hole in the center.

FIGS. 9 and 10 show two groups of red blood cells in which inclusions have formed in their cytoplasm where inclusions appear to be matter removed from leukemia cells.

FIG. 14 is a blood sample plate from a 59 year old female having chronic lymphocytic leukemia.

FIG. 15 is a blood sample from the same patient taken within two hours of the FIG. 14, during which time the patient was administered vaccines prepared in accordance with the method of the present invention halving of leukemia count is evident.

FIG. 16 is a blood sample plate from a 24 year old male having granulocytic leukemia.

FIG. 17 is a blood sample from the same patient taken 7 days later, during which time the patient was treated with vaccines prepared in accordance with the method of the present invention showing remission.

FIG. 18 illustrates 2 leukemia cells in proximity to the red blood cells and other blood constituents.

FIG. 19 shows total lysis of leukemic and red blood cells within minutes of addition of antiserum raised against the leukemia cells.

FIG. 20 shows a leukemic cell surrounded by red blood cells.

FIG. 21 shows coccal organisms in a sarcoma biopsy. Antisera raised against these are used to treat the leukemia (also a sarcoma) blood in FIG. 20.

FIG. 22 shows lysis of cancer cytoplasm, membrane and nucleus with no harm to surrounding red blood cells.

FIG. 23 illustrates both precision and potential of this technology in a leukemia cell treated by antiserum raised against genetic and other fragments of associated organisms inducing removal of cancer genetic component.

FIG. 24 shows a large breast cancer with central ulceration as seen in the mammogram of a 72 year old female.

FIG. 25 is the same patient showing dramatic reduction in mass after 2 weeks of therapy. Arrows indicate cancer margins in FIGS. 24 and 25.

FIG. 26 is of a squamous cell carcinoma indicated by the arrow, stretching to the apex of the right lung.

FIG. 27 shows collapse of that mass within 2 weeks of therapy.

FIG. 28 is of a bone scan demonstrating prostate cancer metastases. These can be seen as the dark marks on the ribs indicated by the arrows.

FIG. 29 shows drastic resolution of the rib lesions following 3 weeks of therapy.

FIG. 30 shows brain metastases in the right hemisphere from a small cell carcinoma.

FIG. 31 demonstrates the lung primary referred to above and its mediastinal spread.

FIG. 32 represents a CAT scan of breast cancer metastasized to the left lung with mass and fluid marked by the arrow.

FIG. 33 demonstrates resolution within 5 weeks of therapy.

FIG. 34 is of an adenocarcinoma of the breast.

FIG. 35 demonstrates a metastasis from breast cancer into the liver.

FIG. 36 demonstrates resolution of breast cancer after 2 weeks of therapy.

FIG. 37 is of a primary hepatoma perforating the right hemidiaphragm and surrounding the right lung.

FIG. 38 shows cancer eliminated from the right lung field.

FIG. 39 is of a adenocarcinoma of the breast in a 42 year old female as shown by mammogram.

FIG. 40 shows massive shrinkage after 4 weeks of therapy.

FIG. 41 shows a large mass obstructing the esophagus in a male age 60 suffering from esophageal cancer marked by the arrow. Patient is unable to swallow food or water at this stage.

FIG. 42 shows that after only 4 weeks treatment the cancer shrunk massively. Patient's esophagus is patent, and he is able to eat and swallow easily.

FIG. 43 illustrates giant cell lymphoma in a 32 year old female, 16 cm in diameter as measured on chest x-ray.

FIG. 44 shows the same tumor after 1 week of treatment shrunk to 3 cm.

### DETAILED DESCRIPTION OF PREFERRED EMBODIMENTS

Definitions

Various terms used herein are defined as follows:

Agent: See Disease-Associated Agent.

Antagonistic Agent: A biological, chemical or physical agent which carries anti-disease activity, such that a regression, whether partial or complete, of the targeted disease may be stimulated by the activity. As used herein, the term “antagonistic” refers to the activity of an agent toward a targeted disease, rather than the effect said agent may have upon the host organism. Such antagonistic or anti-disease activity may be either direct, affecting a targeted disease via contact with the disease or diseased tissue, or indirect, stimulating immunological or other disease-inhibitive activity in a host, and thereby indirectly affecting the targeted disease. Such direct anti-disease activity may include cellular membrane-to-membrane contact between a chemical or biological antagonistic agent and a disease or diseased tissue, ingestion of portions of disease cells or diseased tissue by an antagonistic agent which is an organism (an “antagonistic organism”), direct bombardment of a disease or diseased tissue by a physical agent which is radiological in form, or the like. Antagonistic agents may act indirectly against a targeted disease by precipitating immunostimulant activity beyond that which has been already activated in a host's response to the targeted disease. Additionally, an antagonistic agent may stimulate other indirect anti-disease activity, including changes in the host which are physiological, metabolic, biological or chemical in nature. Such changes tend to help antagonistic agents which compete with the targeted disease for substrate, nutrition, or metabolic products. An antagonistic organism may further secrete compounds which may inhibit disease metabolism, function and growth. The term “antagonistic” has also been used broadly herein, to refer to both antagonistic and nemesis agents (see Nemesis Agent, below).

Antagonistic Organism: An antagonistic agent which is an organism. Antagonistic organisms may further include organisms, extracts and modifications thereof, which are normally independent of a patient, such as targeted disease antibodies raised in a non-patient-specie host, or which are normally found in the patient, such as various components of a patient's immune system.

Beneficial Agent: A biological, chemical or physical agent which may demonstrate causative or synergistic activity toward a targeted disease. As used herein, the term “beneficial” refers to the activity of an agent toward a targeted disease, rather than the effect the agent may have upon the host organism. Such beneficial or pro-disease agents may also include those which are infective toward a targeted disease, if, for example, the overall result of the infection is that the effectiveness of the host organism's immune response against the disease is lessened as a result of dealing with by-products of the infective agent.

Beneficial Organism: A beneficial agent which is an organism. Beneficial organisms may further include organisms, extracts and modifications thereof, which are normally independent of a patient, or which are normally found in the patient.

Biological Agent: See Organism.

Causative Agent: A biological, chemical or physical agent which causes or evolves a targeted disease under specified conditions.

Causative Organism: A causative agent which is an organism. Causative organisms may further include organisms, extracts and modifications thereof, which are normally independent of a patient, or which are normally found in the patient.

Detrimental Agent: A biological, chemical or physical agent which exhibits a negative affect on a targeted disease. As used herein, the term “detrimental” refers to the activity of an agent toward a targeted disease, rather than the effect said agent may have upon the host organism. Such detrimental or anti-disease agents may include antagonistic agents, infective agents and nemesis agents.

Detrimental Organism: A detrimental agent which is an organism. Detrimental organisms may further include organisms, extracts and modifications thereof, which are normally independent of a patient, or which are normally found in the patient.

Different-Specie: A specie other than that of the patient or host organism.

Disease: A sickness, ailment or otherwise undesirable bodily condition. This tern is used rather broadly herein to include naturally occurring bodily conditions which may be undesirable, such as those degenerative conditions occurring attendant to old age, and other pathogenic bodily conditions which may have been caused or otherwise stimulated by non-biological agents. This term is used further herein to include both disease-causative agents and diseased tissue.

Disease-Associated Agent: A chemical, mineral or physical agent, biological organism, extract or modification thereof, which demonstrates at least one of the following associative characteristics or activities regarding a targeted disease: a causative affinity toward the targeted disease; a synergistic affinity toward the targeted disease; a neutral affinity toward the targeted disease; an infective affinity toward the targeted disease; an antagonism toward cells of the targeted disease or diseased tissue; or a regression-stimulating nemesis antagonism toward the targeted disease or diseased tissue. Such disease-associative characteristics may be either naturally inherent in the agent, or may be induced in accordance with the method of the present invention. As used herein, this term may further refer to agents which demonstrate at least one of said associative characteristics toward another disease-associated agent, which another disease-associated agent demonstrates at least one of said associative characteristics toward the targeted disease or diseased tissue.

Disease-Associated Organism: A disease-associated agent which is an organism. Disease-associated organisms may further include organisms, extracts and modifications thereof, which are either normally independent of a patient, or which are normally found in the patient.

Disease-Specific Agent: Another term for disease-associated agent.

Host: An organism in which is located either a disease, diseased tissue or a disease-associated organism or condition we wish to alter. At times, this term is used herein as another term for “patient.”

Infective Organism: An organism which lives within a host in an infective relationship to a targeted disease, which relationship may be either harmful to the targeted disease (in which state it is antagonistic toward the targeted disease), beneficial to the targeted disease (in which state it is synergistic toward the disease), or neutral to the targeted disease. For example, Escherichia coli (or “E. coli”) may infect a cancer and cause breakdown of diseased tissue; however, the toxins which are produced from such breakdown may cause further weakening of the host immune system, which weakening may result in assisting the disease process. The E. coli is, therefore, indirectly synergistic toward the disease (and antagonistic toward the host) its infective relationship to the cancer; unless, for example, toxins produced by the E. coli interacting with the cancer can be prevented from affecting the host and E. coli and toxin exert activity only locally in the cancer.

Neutral Organism: An organism which may live within a host in a relationship to a targeted disease, which relationship is apparently neither beneficial nor harmful to the disease. For purposes of brevity, this term is also used in the ensuing description in reference to both neutral and infective organisms. Neutral organisms may further include organisms, extracts and modifications thereof, which are normally found independent of a patient, or which are normally found in the patient.

Nemesis Agent, or Nemesis Antagonistic Agent: A biological, chemical or physical agent which carries curative anti-disease activity. Nemesis agents or factors can often overlap with those of other categories. Similar to the activity of antagonistic agents, such anti-disease activity can be either direct, affecting a targeted disease via direct contact with the disease, or indirect, stimulating other activity in a host which detrimentally affects the targeted disease. Features of nemesis agents which may be shared with antagonistic agents further include the ability to precipitate host immunostimulant activity beyond that which has been already activated in response to the targeted disease. Additionally similar to antagonistic agents, nemesis agents may stimulate other activity against a targeted disease. A distinguishing feature of nemesis agents which are organisms is their high affinity for disease-causing organisms or the disease process. Nemesis organisms may secrete compounds which inhibit disease metabolism, function and growth. Nemesis organisms may be used as a source for a wide variety of biological and chemical disease-associated antagonistic agents.

Nemesis Organism: A nemesis agent which is an organism. Nemesis organisms may further include organisms, extracts and modifications thereof, which organisms are normally independent of a patient, or which are normally found in the patient.

Organism: The ensuing description uses this term in two senses. When reference is made to types of organisms, the term is meant to include all forms of life composed of mutually dependent parts that maintain various life processes, including animal, plant and microscopic life forms. When reference is made to the creation of various therapeutic agents from an organism or organisms, the intention is that any and all components or forms of the organism or organisms in question may be used in such creation, including, but not limited to, the whole organism, extracts or fractions of the organism, modifications of the organism or products of the organism.

Patient: An organism in which is located a disease that is targeted for therapeutic treatment. A patient may be a human, an animal, a plant or other organism.

Patient-Specie: Same specie as the patient.

Raise: The action of isolating antibodies or other organisms which have an affinity toward, or are otherwise antagonistic toward, a particular disease or disease-associated organism, extract or modification thereof. Said isolating may include various in vivo and in vitro techniques which are known or which are otherwise described herein.

Synergistic Agent: A biological, chemical or physical agent which carries activity that is beneficial to a targeted disease. As used herein, the term “synergistic” refers to the activity of an agent toward a targeted disease, rather than the effect said agent may have upon the host organism. A synergistic or pro-disease organism lives within a host in a beneficial relationship to a targeted disease, either within, or at a distance from, disease cells or diseased tissue. Such organisms include those which are usually susceptible to the immunological system of a host when the targeted disease is not present in the host, but which live with impunity with regard to the host immunological system when the targeted disease is present in the host.

Therapeutic Agents: A biological, chemical or physical agent which demonstrates certain therapeutic and other beneficial activity toward a host organism when applied to the organism, including the prevention, diagnosis, treatment, prognosis, and monitoring of a targeted disease. Such other beneficial activity may include the enhancement of various life functions, biological systems and processes of the organism.

The inventor has developed a unique method for identifying and using various disease-associated agents for the production and use of a wide spectrum of therapeutic remedies. Such remedies include the use of therapeutic agents produced from various biological organisms, organism extracts or modifications thereof, which organisms have demonstrated at least one of the following disease-associative types of activity regarding a targeted disease or other unwanted bodily condition: causative activity regarding the targeted disease; synergistic activity toward the targeted disease; neutral activity toward the targeted disease; infective activity toward the targeted disease; antagonistic activity toward the targeted disease; or nemesis antagonistic activity toward the targeted disease. The method of the present invention further includes the use of biological, chemical or mineral agents, such as extracts and products of both biological and synthetic processes, and physical agents, such as radiant energy, which have demonstrated substantially similar disease-associative activities or characteristics regarding a targeted disease. Causative and synergistic agents are beneficial to a targeted disease, while antagonistic and nemesis agents are detrimental toward a targeted disease. Although neutral and infective agents may appear to be neither beneficial nor detrimental toward a targeted disease, they may be ultimately either beneficial or detrimental toward the disease, depending on how the effect they have on the targeted disease balances with the overall operation of the host system. Each of these categories of disease-associated agents is listed in the table, below, from left to right, in order of antagonism toward a targeted disease, from those agents demonstrating the least antagonism to those agents demonstrating the greatest antagonism.

| | | | | | |
|---|---|---|---|---|---|
| Causative | Synergistic | Neutral | Infective | Antagonistic | Nemesis |

Causative agents evolve the disease, while nemesis agents carry activity against the disease of such a nature that the activity is curative.

The present invention further provides for the use of such disease-associated agents in the prevention, diagnosis, treatment, prognosis and monitoring of a targeted disease or other unwanted bodily condition.

The method of the present invention provides for a high degree of specificity with regard to the effects of various therapeutic agents upon selected cellular material in a host, due to the use of a microbial affinity between the therapeutic agents and a targeted disease or other cellular material. Such microbial affinity may be either natural or induced. The method of the present invention further provides for an extremely high degree of therapeutic efficacy, due to the use of living organisms in the creation of therapeutic agents, and to the use of other disease-associated agents whose activity toward a targeted disease can be selected for providing a degree of synergism with other therapeutic agents which has been heretofore unknown.

One embodiment of the method of the present invention comprises the following steps: 1) conducting a spectrum search for various potential disease-associated agents which exhibit at least one of the fore-mentioned associative characteristics regarding a targeted disease; 2) testing each potential disease-associated agent found in step 1) to gauge the type and level of its activity toward the targeted disease; and 3) preparing a therapeutic agent using at least a disease-associated agent.

In accordance with the method of the present invention, the activity of such disease-associated organisms and other agents related to a targeted disease is first identified and then classified into one of the aforementioned associative characteristics. Such identification and classification is accomplished through in vitro or in vivo evaluation of various potential disease-associated agents. Simple models may be designed in vitro to qualify and quantify agents in relation to disease association per given conditions. One simple example of this is the counting of leukemia cells suspended in trypticated soy broth and evaluation of viability after addition of various agents. It may be possible to demonstrate that under such conditions certain staphylococcal phase lysates may cause amplification of numbers and greater cellular viability, whereas others decrease number and/or viability.

Some fungi added to the leukemia suspension will produce agents lethal to the cancer cells. It can therefore be seen that such in vitro systems can address any targeted disease (cancer, bacteria, virus, etc.) and qualify the relationships of other organisms and agents to it by gross measures of survival and numbers; more refined measures such as those of metabolism and infective/invasive aggression may also be evaluated in vitro; however, in vivo testing is more indicated in evaluation of absolute disease association definition. The statement that a certain organism synergizes with or supports a disease, for example, can best be demonstrated by augmentation or aggravation of disease condition upon introduction of organism into the living system. Agents may differ in their in vitro and in vivo classification depending on host effects. Certain chemotherapeutic agents, for example, may cause total cancer destruction in vitro and therefore be classified as nemeses but have strong or even stronger effects against the host and therefore may be in vivo classified as synergistic to the disease by its secondary deleterious effects against the host. There are several living systems where application of chemotherapy may predispose to the generation of cancer in which case it may be termed a causative agent or at least involved in the causative process. Various therapeutic agents with attendant treatment pathways and protocols are then prepared from the disease-associated agents based on the classification. Further in vitro and in vivo evaluation of the various therapeutic agents may be conducted for determining which such agents, pathways, protocols, or combinations thereof, may work best for a particular patient.

Also, such further in vitro testing of the therapeutic agents can provide an accurate basis upon which a prognosis concerning the targeted disease may be made, because many therapeutic agents produced according to the method of the present invention do not depend on stimulating the patient's immune system, but rather have an inherent ability to attack a targeted disease directly, apart from any assistance which may be available via elements of the patient's immune system. For example, in accordance with the instant invention, if a disease-associated organism is classified as being detrimental toward a targeted disease (i.e., the activity of the organism is infective, antagonistic or nemesis antagonistic toward the targeted disease), a form of said organism is then isolated for effective use in targeting the disease. A therapeutic agent is made from the isolated antagonistic or nemesis organism, which agent enhances the patient's existing defensive response against the disease via adding its independent anti-disease activity to the patient's body.

An example here may be made of the use of phages and their use as nemesis organisms in the treatment of bacterial infection. A host suffering of staphylococcal infection, for example, may have such organism cultured and nemesis phages may be identified from stock cultures or from the same staphylococcal culture by exposing it to stress in the form of metabolic (nutrition), oxidative (peroxide, ozone), or any other physical, chemical or biological stress including UV light exposure amongst many other known processes or those to be discovered. The phage isolated will exist in vitro as a nemesis in that phages will be found that will destroy almost all of the bacteria in culture. Phages have been used previously as antibiotic agents but largely were not as effective in vivo. This was at least in part due to the lysogenic potential of many phages despite initial lytic activity and the ability of bacteria to develop immunity to infection. The inventor has created a lysogenic coefficient indicating likelihood and time needed for lysogeny to develop between certain phages and bacteria as well as mechanisms designed to deal with bacterial resistance which include use of pharmaceutical agents such as antibiotics to deal with resistant bacteria and maximize destruction of those sensitive to phages, specific antisera can also be used to do the same and plasmid therapy to mark the bacteria antigenically for antisera etc. and maintain or induce bacterial sensitivity to phage or other therapy. A major difference between this and prior art is the use by the author of lytic phages without significant lysogenic potential so that lytic effect is maintained. Samples of disease are also taken regularly to gauge phage and other sensitivities.

Phages also may be used to prepare and augment immune response to the phage-lysed bacteria by introducing the phage-lysed bacterial fragments into the body so that it may anticipate it and respond strongly to the challenge; phages and phage lysates also are strong immune stimulants and therefore may have both direct anti-disease activity as well as secondary characteristics beneficial to host. Prior preparation of the host against the lysed bacteria and use of multiple generations of lytic phage, the use of plasmids and phage to confer required properties to the target organisms or to prevent the acquisition of unwanted ones along with the use of antibiotics, antisera, etc. (including the use of transfer factor, a cellular extract that can educate the host's immune response), patent introduces the concepts of anticipation (preparing the host for a change about to arise as natural progress of disease or subsequent to treatment), multiphasic therapy where stages of treatment are planned according to resistance that arises in disease; e.g., the use of multiple phases of phage designed to deal with anticipated or actual resistance that develops following the use of each solely or in combination. The invention also introduces multimodal or polyvalent therapy alone or in combination with the polyphasic and anticipatory models to optimize therapeutic efficacy.

Those skilled in the art will recognize that this type of therapy may be significantly more advantageous to a patient than current therapies which merely attempt to stimulate the patient's immune response by vaccinating the patient with fractions of a targeted disease. If, on the other hand, a disease-associated organism is found within the body of a patient, which organism is classified as beneficial regarding a targeted disease (i.e., the activity of the organism is either causative or synergistic toward the disease), a therapeutic agent which is antagonistic toward such beneficial organism may be administered to the patient for aiding the patient's immune system in fighting against such causative or synergistic organism. Organisms have often been reported to exist in cancer patients both in tumor biopsies and in overt disease, in the treatment of a non-Hodgkins lymphoma patient suffering from mixed bacterial pneumonia, inventor prescribed broad spectrum antibiotic cover-Penicillin 1 million units three times a day, chloramphenicol 1.25 g three times a day, and flagyl 400 mg three times a day; the first two by intramuscular route and the flagyl orally. Following one week cancerous lymph nodes shrank and softened suggesting that the antibacterial action of the antibiotics was having anti-cancer activity.

Similar protocols have not altered disease conditions in other patients of same cancer type, suggesting different organism targets in different individuals. Indeed, it has been found that challenging such causative or synergistic organisms often results in simultaneously challenging the targeted disease. Again, the patient's ability to fight the disease is enhanced by administration of a therapeutic agent, such as an anti-causative or an anti-synergistic organism, rather than by the attempted stimulation of what may be an already weakened or suppressed immune response. Although practicing the method of the instant invention involves the application of extracts or other modifications of organisms which are normally pathogenic toward a host of the patient's specie (e.g., they are fractions of another disease), such application is not generally accomplished without the additional application of other disease-associated agents or organisms which are capable of enhancing the patient's own anti-disease response beyond that which is achievable via merely stimulating the patient's immune system with fragments of the targeted or a similar disease. For example, in the case where disease fractions are administered to a patient in accordance with the method of the present invention, it is generally preferred that such disease fragments are either nonpathogenic toward the patient, or that other disease-associated agents or organisms or parts thereto which are antagonistic

toward the disease fractions are subsequently administered to the patient, for neutralizing the pathogenesis of such disease fractions after they have accomplished their intended purpose.

The instant invention further involves manipulation of the affinity between a disease-associated agent and a targeted disease in order to increase the specificity and decrease the toxicity of therapeutic agents made from such disease-associated agents. The method of the present invention also includes the use of disease-associated agents already having a high degree of affinity toward a targeted disease and further includes increasing or enhancing the affinity toward a targeted disease. Still further, the method of the present invention includes inducing such affinity in organisms which initially demonstrate no affinity toward a targeted disease.

The method of the present invention includes decreasing such affinity between a disease-associated agent and other cellular material, which material the therapeutic agent to be derived from the disease-associated agent is not intended to affect. The inventor has found that such manipulation of affinity between disease-associated agents and a targeted disease or other bodily material can significantly increase the specificity of therapeutic agents which are ultimately derived from such disease-associated agents. Simultaneously, there is a decrease in the toxicity of such therapeutic agents, which often results in a significant decrease in the occurrence of undesirable side-effects. As well as increasing the affinity of a disease-associated agent toward a targeted disease, the method of the present invention may also be used to increase the efficacy of various antagonistic and nemesis disease-associated agents by increasing the antagonism of such agents toward a targeted disease, and even stimulating the antagonism in disease-associated agents which initially demonstrate no such activity toward a targeted disease. It has been found that such antagonism enhancing can be accomplished even with components of a patient's own immune system.

As well as manipulating the affinity or antagonism of a disease-associated agent, and even stimulating such characteristics in a disease-associated agent which has not yet demonstrated such activity, the inventor has found that various other characteristics may be transferable between various disease-associated agents and other organisms or agents. Indeed, the method of the present invention further includes transferring various desirable characteristics from a first organism to a second, in order to biologically enhance the second organism. It has been demonstrated that such biological enhancement may be beneficial in assisting an organism in recovering from the ravaging effects of a disease. It has been demonstrated further that such biological enhancement may be beneficial in adding whole new dimensions of capability to an organism.

The method of the present invention further makes use of the affinity of disease-associated agents toward a targeted disease by using disease-associated agents as tagging mechanisms or tagging agents. The inventor has found that the tagging agents may be useful not only for making a targeted disease more visible to a patient's immune system, but that they may be useful also for making the disease more visible to other disease-associated agents or anti-disease factors, which other agents or factors do not have as great an affinity toward the targeted disease and, therefore, may be directed to the targeted disease indirectly via their attraction to the tagging agents. One approach to accomplishing this includes the use of a tagging agent to carry an anti-disease agent with it to a targeted disease. Alternatively, other disease-associated agents may be used which are antagonistic toward the tagging agent, antagonistic toward the tagging-agent-and-disease complex, or antagonistic toward products of the complex for challenging the disease after such tagging agents are in place.

The method of the present invention also provides for the use of radiant energy generating devices in the prevention, diagnosis, treatment, prognosis and monitoring of a targeted disease. The inventor has found that various body tissues resonate at various natural frequencies, which can be identified, enhanced, and monitored. It has been found that tissue which has been affected by disease cannot carry the same dominant frequency patterns as its healthy counterpart. The inventor has developed several devices which capitalize on this phenomenon in treating a targeted disease.

Various embodiments of the invention are described in greater detail, below.

### EXAMPLE 1

Therapeutic agents are produced from organisms, organism extracts or modifications thereof, which demonstrate detrimental activity toward a targeted disease (i.e., the detrimental organisms are antagonistic or nemesis organisms). The organisms may include those which have demonstrated a natural antagonism toward a targeted disease, as well as those in which such an antagonism is induced or raised in accordance with the method of the present invention.

The initial step in producing a therapeutic agent is to perform a spectrum search for potential disease-associated organisms, which demonstrate such detrimental activity. The spectrum search should include an epidemiological search for organisms which are known to exist in geographical areas having a low incidence of the targeted disease. It can be deduced that some disease-inhibiting element or organism exists within the low-diseased areas, and may be identified for use in treating the disease. A wide range of organisms should be reviewed for the search, including bacteria, viruses, fungi, parasites and plants. Correlating the incidence of viral, bacterial, fungal, parasitic and other types of infections with the incidence of the targeted disease on a global geographical basis will yield further information on potential detrimental organisms (suggested by the high incidence of these organisms and their clinical manifestations with the scarcity of the targeted disease). For example, in geographical areas having a high incidence of malaria, cancer is scarce. The incidence of cancer is also known to increase in such areas where malaria is eliminated. This suggests a possible antagonism between malaria and cancer, thereby qualifying malaria as a potential disease-associated organism with regard to cancer.

Also, non-geographical statistical surveys should be performed to identify potential disease-associated organisms which are not normally affiliated with specific pathogenic states. Such non-affiliation may indicate an antagonistic or nemesis activity between such organisms and the pathogenic states with which they are not affiliated. Examples of diseases and microorganisms which have such statistical indications of an antagonism toward various cancers include syphilis (treponema pallidum), measles, mumps (and measles and mumps viruses), and staphylococci often in skin infections, erysipelas (streptococci), tuberculosis (mycobacteria), malaria (plasmodia)—all may in whole or in part possess direct or indirect action and are potential sources for anticancer therapeutic agents. Still other statistical information indicates that cardiovascular disease and cancer are opposing maladies, in that the incidence of one in an individual reduces the likelihood of the other also occurring. Initial work by inventor suggests that similar organisms or mechanisms may be associated with both; cholesterol and triglyceride levels with cancer patients receiving therapy as per patent would usually improve dramatically. Applying the concepts and therapies described here in whole or part drastically reduced cholesterol levels and often reversed cardiovascular disease to some degree.

One particular phenomenon which should be considered during this part of the search is that of biological interference, wherein organisms of the same specie (i.e., virus to virus, bacteria to bacteria, fungus to fungus, cancer to cancer, etc.) normally interfere with each other's ability to survive in a shared host. For example, it is extremely rare for a single individual to be infected with two viruses or two cancers simultaneously. It has also been found that certain plant and animal viruses when used in accordance with the present invention can negatively impact the HIV virus. Indeed, the use of certain organisms like types of Staphylococci and their or other phages in the method of the present invention significantly interferes with the HIV virus. Preparations tested cause considerable interference both with viral replication as well as its ability to destroy T-cells. In a study of ten AIDS patients treated with vaccines made from staphylococcal phage lysates (10 to the 9 plaque forming units per ml; 2.5 cc administered intramuscularly) 8 showed 10 fold drop in their viral load as measured by PCR analysis and greater than 50% increase in their T cell counts over the same time period. Still further, it has been found that some tumors elicit strong responses against unrelated tumors. Accordingly, if the targeted disease is a cancer, the spectrum search should also include a review of other cancers as potential disease-associated organisms, since most cancer hosts carry only one form of cancer.

Regarding concerns about treating one disease with another which will ultimately also need treating, the inventor has discovered that use of non-disease tissue from a host infected with the corresponding disease can carry sufficient anti-similar-disease factor for use as therapeutic agents against the similar targeted disease. For example, it has been found that therapeutic agents made from the ascitic fluid of a breast cancer patient may exert very powerful anti-cancer activity against prostrate cancer, as indicated by the pronounced shrinkage of tumor masses within minutes to hours of application in accordance with the method of the present invention. Ascitic fluid may provide a bank of, amongst other things, human anticancer antiserum. Inventor has found dramatic anticancer activity in others of similar disease and even moreso in some cases of different cancers in vitro, ascites from breast cancer may cause a 100% cancer cell kill in lines of leukemia and melanoma, for example, in vivo cancer-related fluid (some activity has also been observed by author where fluid in tissue space such as pleura or peritoneum have been of cardiac, hepatic or other origin) needs some caution in use as carcinoma-related fluid may cause initial improvement followed by aggravation in sarcoma-bearing animals and vice-versa.

It is preferred that the epidemiological search for potential disease-associated detrimental organisms be a continuous effort for the purpose of creating a library of potential disease-associated organisms which can be readily accessible for therapeutic use against a wide variety of pathogenic agents. Antagonistic organisms which are identified in other aspects of the spectrum search, as described hereafter, should also be added to this library for therapeutic use with future patients having a similar disease. In the inventor's development of such a library, he has found many organisms, including disease-associated extracts and components of host immune responses, which are readily useful in the creation of therapeutic agents against diseases and other non-desirable bodily conditions that have been heretofore classified as incurable. For example, penicillin's usefulness as an anti-bacterial therapeutic agent is well known. By adding the parent organism Penicillium notatum to the library for identifying other potential uses of this organism, the inventor has discovered that enzymes extracted from Penicillium notatum have an apparent therapeutic effect upon the HIV virus. Penicillium notatum cultured whole with or without subsequent physical/chemical or biological lysing exerted 65% inhibition of viral growth in T-cell culture over 48 hours. With specific culture characteristics, to be described in patent, inhibition was raised to 99%; other penicillia and yeasts and fungi are also capable of this.

The spectrum search for potential disease-associated detrimental organisms should also include a search for organisms which may be found in vivo. An in vivo search should include looking within a patient-specie host, especially such a host which is undergoing a remission from the targeted disease or a disease similar to the targeted disease. A blood sample or a biopsy of diseased tissue from the remissive host will likely reveal an organism which is actively attacking the disease, attacking another agent that has “tagged” the disease, attacking a complex comprising a tagging agent and diseased tissue, attacking by-products of such a complex, or attacking another organism that is in a synergistic relationship to the targeted or similar disease. A search in the remissive patient's blood, nose, throat, ears, ascitic fluid, urine and stool should also be made for organisms not normally associated with the patient's body, paying special attention to those organisms for which no apparent immunological activity has been precipitated.

The spectrum search for potential disease-associated detrimental organisms should further include a search for organisms which may be found in vivo within a patient-specie host which is infected by a different disease, which has demonstrated an antagonism toward the targeted disease, as indicated in prior geographical or statistical correlation, or as may be indicated as a result of anticipated biological interference. It has been found by the inventor that effective sources of antigens and disease specific antibodies exist in ascites, plural effusions and other tumor effusions of remissive patients and of still other patients having a similar, or otherwise biologically interfering, disease along with a factor of accelerated cancer cell death, assumed to be but not restricted to a genetic code, possibly a ribonucleic acid termed dsRNA (referring to death sequence RNA). In reality, several death sequence compounds may be defined including enzymes and co-factors. DsRNA was postulated to account for the rapid, non-inflammatory cancer cell death seen upon addition of such fluid to cancer cells in vitro and in vivo. It appears to exist in higher concentration where the fluid is not cell-free. Contents of such fluid also appear to have strong antimicrobial, anti-inflammatory and regenerative capacity. Preliminary work even suggests the possible use of cancer fluid and cancer cell extract in the increase of cellular division beyond the Hayflik limit. Death sequence agents may have counterpart life-sequence agents and may vary in their role depending on target. They may represent the cellular version of bacterial phages and plasmids. Preliminary animal studies point to the possibility of using these agents in disease prevention, treatment and life-extension (death sequence nay represent or promote life sequence in other circumstances and/or targets.

Ascitic fluid from a carcinoma or sarcoma patient has been found to have general anti-cancer activity. In humans, for example, it has been found that therapeutic agents comprising sterile human effusions or ascites from such other human hosts are not only effective against various diseases, but induce only a few side-effects when administered intramuscularly, subcutaneously, intradermally, or intratumorally with doses up to and exceeding 150 cc. It is further believed that treating a patient with a therapeutic agent made from an identical cancer type from another patient-specie host in accordance with the present invention may stimulate the patient's immune system against the foreign tissue. In so doing, the patient's immune system may be induced into recognizing its own cancer cell material which has been previously “hidden” from the immune system as being similar to the foreign material and, thus, attacking the previously hidden cancer cell material.

Rejection of foreign, implanted tissue by the host body has been a long-studied immunologic phenomenon and a dreaded consequence of transplant surgery. Recent success in transplantation has been in part due to better matching of donor but largely due to application of immunosuppressant technology and agents. There has been minimal, if any work on the use of such rejection mechanisms in a therapeutic format.

There have been many attempts to raise an immune response against cancer by various genetic or membrane manipulations to alter its structure to one more antigenic or to secrete/express immune-attracting complexes.

Occasionally, spontaneous remission follows some localized or systemic infection and this has led to the use of viral/bacterial/other micro-organism preparations in the treatment of cancer. Those of note include Coley's toxins, the Newcastle's disease vaccine as well as the recent use of Herpes viruses to sensitize and target cancer cells. There have also been researchers who have claimed that certain organisms cause cancer and designed vaccines to deal with them such workers have included Glover, Scott as well as Livingston in more recent times. The latter two recommended the use of killed “causative” organism vaccines whereas Glover actually developed a specific antiserum-his approach is probably more logical in view of the “clonal depletion” section of this patent (but as all others, limited by one causative target).

Viruses, bacteria and other microorganisms eliciting major immune responses tend to be too deadly for use whereas others are often capable of causing a temporary response but often become incorporated in the matrix of the cancer cell and directly or indirectly aggravate its growth. During the 50's and 60's many researchers experimented on patients with a plethora of viral and other microbial agents to attempt to treat a wide array of cancers. The results never matched the spectacular responses occasionally reported after random infection. The pattern of remission events, the phenomenon of reactivation and one of the most powerful immunological phenomena, graft rejection was to suggest some answers to this apparent paradox.

Remissions from cancer had been observed following a range of viral and bacterial as well as other micro-organism infections (malaria, trypanosoma cruzi, syphilis, etc.). Bacterial and higher organisms have been considered in other parts of the patent, most of the work done with the higher organisms has concentrated on preparations and extracts of the whole and therefore, the experimental situation could not be said to mimic the live infection scenarios; viruses, however, have been used in their entirety in many trials. The culturing and purification of viral organisms and the attempt to increase their efficacy, specificity, etc., by standard culture methods (unlike those of the instant invention) as well as others and their use in ever-increasing concentrations usually failed t duplicate their reported success as a natural event. Even the use of steroids to immunosuppress the patient during the infective phase to allow for optimal viral infection of cancer cells failed to improve efficacy. The paradox, therefore, was that the remissions observed occasionally with clinical viral infections could not be equaled, let alone surpassed by the use of higher infective dose and controlled immunosuppression to allow for higher viral saturation of cancer.

Resolution of the paradox lay in that the occasional tumor-resolving effect of viral infections could not simply be attributed to pure viral effects of pure antiviral immune or other defense mechanisms. Natural infections also did not always result in automatic remission. Other factors must have been involved.

Work in the mid-70s (1974, Teruo Asada, cancer 34:1907-1928) yielded promising results in the use of mumps in the treatment of breast and other cancers. The virus was isolated fresh from human donors and introduced into the patient. Tumor regression occurred within days to weeks. Attempts by the author to duplicate the work using attenuated vaccine failed to yield any positive response. Clearly, freshly donated virus carried factors not contained in the attenuated vaccine. Interestingly, regression occurred even in patients previously immunized or had suffered mumps in their youth. The ability to infect the cancer cells appeared unaffected and/or the infective dose was not an essential factor (explaining the failure of steroid therapy to improve effect).

There appeared to be a fundamental difference in infections acquired naturally which could cause remissions and attenuated forms of the same virus as used in vaccines or repeatedly passed through cancer cells in order to stimulate specificity and efficacy. The latter two preparations share more than just inefficacy. Most attenuated viral vaccines are cultured on either embryonic cells or immortal (cancer) cell lines. This is done so as to minimize the risk of introducing strong foreign antigens by the culture medium, amongst other reasons.

Many infective agents cannibalize fragments of their host and may express them in an antigenically significant form in the initial phase and/or throughout the infection. Cancer cells may also carry other infections or mechanisms for amplification of such antigens once introduced into the system-this may be of assistance but is not an essential requirement of the theorized model; it however, may explain how effect may be elicited by inactive or dead extracts of infection or of foreign tissue it also opens a new realm of possibilities including genetic manipulation of cancer to induce expression of foreign antigens.

The one hallmark, repeatable experiment in cancer immunology is the grafting of cancers across species or even in different members of the same species. The greater the difference genetically between recipient and donor, the more aggressive the rejection phenomenon. Prior art in application of this observation involved the implanting of one person's cancer into another cancer patient. The implant would be quickly reflected and it was hoped that the patient's own tumor would suffer from some form of crossover effect where an angered immune response hopefully recognizes some common “cancer antigen” in both rejected graft and recipient's tumor and then attacks the patient's cancer.

Upon studying cases treated in this manner, it is easy to see where this theory is flawed.

1. There appears to be almost instant destruction of transplant as would be expected from a graft refection yet the patient's own tumor also may undergo rapid breakdown (within hours). This would suggest a much more rapidly activated phenomenon then a crossover effect. Part may be due to dsRNA and other DS factor activity, part may be due to a direct cancer versus cancer activity or even antibodies/other immune response transplanted with tumor. Work earlier this century involving injection of ascitic fluid into cancer patients led to significant tumor regression and improvement both in length and quality of life (Ill and Miningham, 1912; Hodenpyl, 1910). Ascites and other effusions such as pleural cancer-related effusions have more recently been shown to carry significant titres of anticancer antibodies. It is thought that in cell-suspension form that the cancer cells express antigens more openly and antibodies are therefore easier to manufacture (non-cancer effusions, however, also seem to carry anticancer activity). It appears that ascitic fluid from long term survivors carried more beneficial effects than that isolated from others. in vitro data demonstrated that several ascitic fluids (breast, bowel, hepatoma) could destroy cancer cell cultures; both leukemia and melanoma cells were tested at the University of Colorado School of Medicine.

Work by inventor demonstrated that in vivo results were best if the fluid used was from the same broad group of sarcoma or carcinoma as the treated animal. Despite efficacy of fluid from either source against a wide range of cancers from both classifications in vitro; treatment of sarcoma with carcinoma fluid or vice-versa met with very transient alleviation of symptoms followed by rapid growth and death of animal. The relative shortage of sarcoma fluid allowed for heavier documentation of catastrophe following sarcoma treatment by carcinoma fluid in view of the in vitro findings, it appeared that sarcoma cells and associated fluids are capable of destroying carcinoma cells and vice-versa to at least equal efficacy as various carcinomas and related fluids and sarcomas and related fluids could. The in vivo data seems to indicate that sarcoma and carcinoma may destroy each other factors lead to shortened life span, perhaps the unchecked release of disease cause. Then a carcinoma-related fluid or a sarcoma-related fluid causes shrinkage of a same-group cancer effects in vivo are longer lasting-and more dramatic. Inventor has also found evidence that interchange of cellular structures is more marked when dealing within the same broad heading of cancer type.

2. Ascites/pleural effusions etc. from a patient who is stable or who is undergoing tumor regression have been shown to exert beneficial action on patients of similar cancers to whom it was administered. It is likely that the mechanism involves antibody responses of some potency in the fluid and one would therefore expect optimal responses from patients with identical tumor types and related antigens.

If the above was the only mechanism involved then the following observation should not be possible; fluid from cancer patients doing poorly also exerted an antitumor effect; most marked when the tumors belonged to the same broad heading of carcinoma or sarcoma but were markedly different within those headings.

A case demonstrating this was the rapid disappearance of a 5 cm diameter skin metastasis from a prostate cancer patient upon application of ascitic fluid from an ovarian cancer patient. Its disintegration and resorption occurred over a few hours. Other, distant sites began to recede over a few days. Ovarian cancer and prostate cancer are not antigenically related in any manner worthy of such a response. it should be further noted that even in absence of ascitic fluid, even washed cancer cells from one patient were able to cause at least temporary tumor regression when injected into another. Part of this may be explained by the Death Sequence Factors as described previously. Even should the theory of spillover from immunological angering be a factor; antigenic stimulation of implanted tumor is greater, the greater the difference from one donor to host (effects have been noted by inventor both with implants of the same of different species, however, same specie implants may have greater spillover effect). Cancer-related organisms from one patient may infect the cancer of the host and may be recognizable antigenically by the new host).

It is also possible that cellular exchange or donation of antigen occurs between implanted cancer and that of the host leading to the cancers both being rejected as foreign. While it is also possible that we are witnessing some cancer versus cancer phenomenon or even antitumor antibody more specific to causative organisms than cancer cell type the more the cancer antigens vary.

There is evidence that cancer-specific antibodies/compounds exist secreted against or even by cancer cells in body fluids. The ability of ascitic fluid to effect cancer breakdown in-vitro without other blood factor indicates a direct effect yet the inability to produce carcinoma fluid versus sarcoma or vice-versa benefits in vivo as opposed to excellent responses in vitro indicates that the antibody component may not be the major therapeutic one.

Work by the inventor demonstrating the efficacy of male versus female tumors and tumor responses suggests that rejection phenomena may be active.

Use of the graft rejection response may help explain the difference in efficacy between natural infection and vaccines attenuated and cultured. Other than the implications of vaccine attenuation on cancer cells (or continuous cell lines as is common practice) leading to weakened anticancer ability (see cancer attenuation factor); natural infections carry antigens of their previous host and upon infecting cancer may express both viral and graft rejection antigens. The following pictures demonstrate a zone of separation between cancer cells and normal tissue suggestive of graft rejection following inoculation of Newcastle's virus in its natural form; suggestive of graft rejection.

Prior art in this field includes the passaging of virus/other microorganisms through cancer tissue cultures to attempt to increase specificity and efficacy. This has usually been either without effect or, worse, induced tolerance, e.g., pushing a virus into a lysogenic form. (See cancer attenuation factor-also discovered by inventor).

A simple explanation for the logic to follow is that when the existing options are attenuation or extinction, synergistic or attenuated forms of the virus will be selected for a system to isolate specific and destructive viruses must therefore provide conditions which promote its survival and propagation. Serial passages through cancer cultures will select for virus that can synergize with the cancer cell unless the capacity for cancer cell lysis is selected for by culture technique serial or continuous passaging or even static culture mechanisms are unlikely to select for aggressive viral strains or amplify them; the viral strain that survives best in this format is one with inherent or acquired cancer synergy and amplification and survival occur best for viruses which do not destroy their cellular host.

One technique for selection of deadly viral particles (DVP) is Timed Culture Techniques (TCT). Theory here assumes that the rate of growth and viral release is higher in aggressive viruses than in synergistic ones. A times aggression coefficient can be defined whereby studies indicate the length of time needed for cell lysis to start and collection of viral particles from culture medium at this point yields high titres of destructive virus. This, however, does not rule our fast replicating synergistic viruses that bud or release without cancer cell destruction.

The above problem is resolved simply by repeated centrifugal separation of lysed cellular body and/or filtration and culture of associated virus. Separation along a centrifugal or sugar or other gradient should separate living cells from dead ones and even fractionate the divisions. Viruses existing within the living cells can also be used as antigen carriers particularly upon cellular stress. This model applies equally well for bacteria as it does for cancer and other cells.

The in vivo portion of the spectrum search for potential disease-associated agents should include isolating organisms or other disease-related factors which are residing within the patient. This aspect of the search should include looking for any organisms which may be found in non-infected portions of the patient's own body. Areas of the body not infected by the targeted disease would strongly indicate the existence of a disease-associated organism or other immune agent, for example, an immune factor produced by a disease-associated organism, or even an immune factor which is produced by the uninfected body portion. As previously noted, there are often many parts of a patient's body which normally remain unaffected by a particular disease.

With regard to cancer in humans, unaffected areas often include the spleen, small intestine and muscular system. It has been found, for example, that Peyers Patches from a human patient or donor may be extremely useful in the method of the present invention, due to the inherent ability of such tissue to withstand a large variety of diseases. It has still further been found that extracts of a muscle biopsy when activated by enzymatic stimulation, biological fortification or amplification by any other means exhibit a strong anti-cancer therapeutic effect. Challenging balb-c mice with mop-c myeloma at the same time as injection of lysed rat muscle extract delayed the period of onset of disease. Furthermore, intramuscular injection of a hypo-osmotic solution also releases some components of muscle cells so that they exert some anti-cancer therapeutic action. If, however, it is not otherwise desirable to investigate such disease resistant areas of the patient's body, areas of another patient-specie organism may be examined for the disease-associated organisms or immune factors.

The in vivo portion of the spectrum search for potential disease-associated detrimental organisms should further include a search for organisms occurring in, or raised in, the bodies of hosts of a different specie than the patient, such as non-human hosts when the patient is human. Different-specie hosts include those which are susceptible to the targeted patient-specie disease, or a similar disease, and may further include those hosts which are known to be resistant to the targeted disease. Although a wide variety of non-human hosts may be useful in this regard, the inventor has found especially useful hosts selected from the group of non-human host orders or families consisting of: Bovidae, Canidae, Cricetidae, Equidae, Felidae, Lagomorpha, Muridae, Primates, Suidae, and Tayassuidae.

A very accessible type of detrimental organism which can be raised in different-specie hosts which demonstrate an immune response to the targeted disease are antibodies created by the host for attacking the targeted disease. Such targeted disease antibodies may be raised in a first non-patient-specie host by inoculating said first host with any targeted disease related elements which have been extracted from either the patient or another patient-specie host that is infected with the targeted or a similar disease. Such extracted disease related elements may include, but should not be limited to, the following: 1) cells of the targeted or similar disease; 2) lysed fragments of diseased tissue (especially that which is from cellular membrane, mitochondria, golgi apparatus, lysosome, or the like); or 3) any disease-associated beneficial or synergistic organisms which may be identified (as discussed below in greater detail).

After being inoculated with such disease related elements, the first different-specie host will raise antibodies against the inoculated disease related elements. After such antibodies to the inoculated disease related elements are raised, sera containing such antibodies should then be extracted from the first different-specie host.

The next step in preparing a therapeutic agent from antibodies raised in a different-specie host is to “wash” any anti-patient-specie factor from the first host sera. A method of conducting such washing is to inoculate a second different-specie host of the same specie as the first host with normal, uninfected cells of the patient or other patient-specie organism. This will raise antibodies to the normal cells of the patient or patient-specie organism, which antibodies can then be extracted and applied in vitro to the first host sera, for precipitating any anti-patient-specie factor out of the first host sera, thereby leaving the anti-disease antibodies in the sera for use as a therapeutic agent for the patient. The remaining anti-disease antibodies may be further washed repeatedly against healthy cells of the patient for precipitating out any remaining anti-patient factor.

An alternative method of washing any anti-patient-specie factor from the antibodies to the targeted disease which were raised in a different-specie host is to inoculate a third different-specie host with the antibodies to the targeted disease raised in the first different-specie host. Such inoculation will transfer memory of anti-targeted-disease activity to the third host immune system. The third different-specie host should then be intentionally challenged at a later date by a second disease which is common to the different-specie, which second disease is also substantially similar to the patient-specie targeted disease. The initial “memory” immune response of the third different-specie host against the second disease will be anti-second-disease activity. However, such immune response may also include anti-patient-specie targeted-disease activity, and may also, therefore, include antibodies or other factors which may be effective as a therapeutic agent prepared against the patient-specie targeted disease. Of course, the actual activity of said immune response antibodies or factors should be tested to determine the specific type and level of its activity toward the targeted disease and the patient-specie before being made or used as a therapeutic agent. The selected antibodies and immune factors may be further washed repeatedly against healthy cells of the patient, for precipitating out any remaining anti-patient factor.

Another alternative method of raising in a non-patient-specie host antibodies to a targeted disease, which antibodies are free of any anti-patient-specie factor, is to inoculate a first non-patient-specie host with normal, uninfected cells of the patient or other patient-specie organism. This will raise antibodies to the normal cells of the patient or patient-specie organism, which antibodies can then be extracted and applied in vitro to patient specie sera which contains targeted disease related elements that have been extracted from either the patient or another patient-specie host that is infected with the targeted disease or a similar disease. This will precipitate any patient-specie factor out of the sera, and thereby leaving in the sera disease related elements which are free of any patient-specie-factor. Some of the sera which has been thus “washed” of patient-specie-factor is then inoculated into a second non-patient-specie host, for raising antibodies to the disease related elements in said sera. The antibodies raised in the second non-specie-host should then be useful for producing a therapeutic agent for applying to the patient against the targeted disease. Such antibodies may be further washed repeatedly against healthy cells of the patient for precipitating out any remaining anti-patient factor.

Raise organisms having a high affinity toward healthy cells either in vitro or in vivo. Once raised, wash such organisms against organisms having an affinity toward a targeted disease to ensure that any affinity between the diseased-cell-affinitive organisms and healthy cells is eliminated prior to introduction of therapeutic agents made from the diseased-cell-affinitive organisms into the patient. Furthermore, the diseased-cell-affinitive organisms, extracts or modifications thereof, may be similarly washed repeatedly against disease cells in order to extract from the sera only those cells which have the greatest affinity toward diseased cells. The diseased cells may then be removed from such cells having the greatest affinity toward the disease via chemical, physical or biological separation methods which leave the diseased-cell-affinitive organisms, extracts or modifications thereof, intact and substantially ready for introduction into the patient. Such diseased-cell-affinitive organisms, extracts or modifications thereof, raised and washed in this manner will have both a high affinity toward the targeted disease and a high specificity regarding the type of cells to which they are attracted. Indeed, it has been the inventor's experience that patients treated with such diseased-cell-affinitive organisms, extracts or modifications thereof, have developed only minimal, if any, side effects.

Once the spectrum search has determined the existence of various potential disease-associated organisms which are detrimental to the targeted disease, tests should be conducted to determine the type and level of activity which exists between the organisms and the targeted disease. Perhaps the simplest tests to conduct are antibiograms and antichemograms, in which various potential disease-associated organisms and chemical agents are blotted on an enriched or minimal media which includes elements of the targeted disease. Such media blotting or petri dish tests will yield results which are both quick and reliable.

Another test which is most helpful in gauging the affinity of potential disease-associated organisms toward a targeted disease is an in vitro reaction test using cell homogenate of the targeted disease in combination with each potential disease-associated organism. Indeed, the inventor has found that the in vitro tests may be a reliable indicator of disease-associated organisms when no other indication of a particular potential disease-associated organism exists, beyond the statistical indication that a particular geographical area may be a potential source of such a disease-associated organism. In such case, a reliable indicator concerning particular disease-associated organisms can be found by preparing antibiograms or antichemograms, or otherwise conducting in vitro reaction tests using cell homogenate of the targeted disease, in combination with dense complexes of organisms as may be readily available in the indicated geographical area. Such organism complexes may include plate soil, manure, compost samples, or the like.

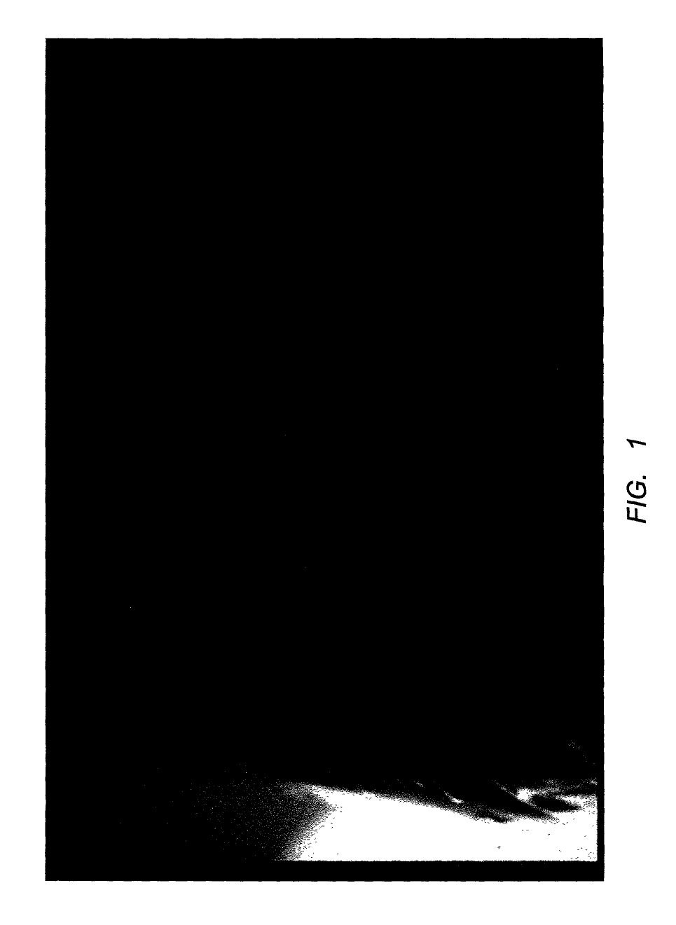

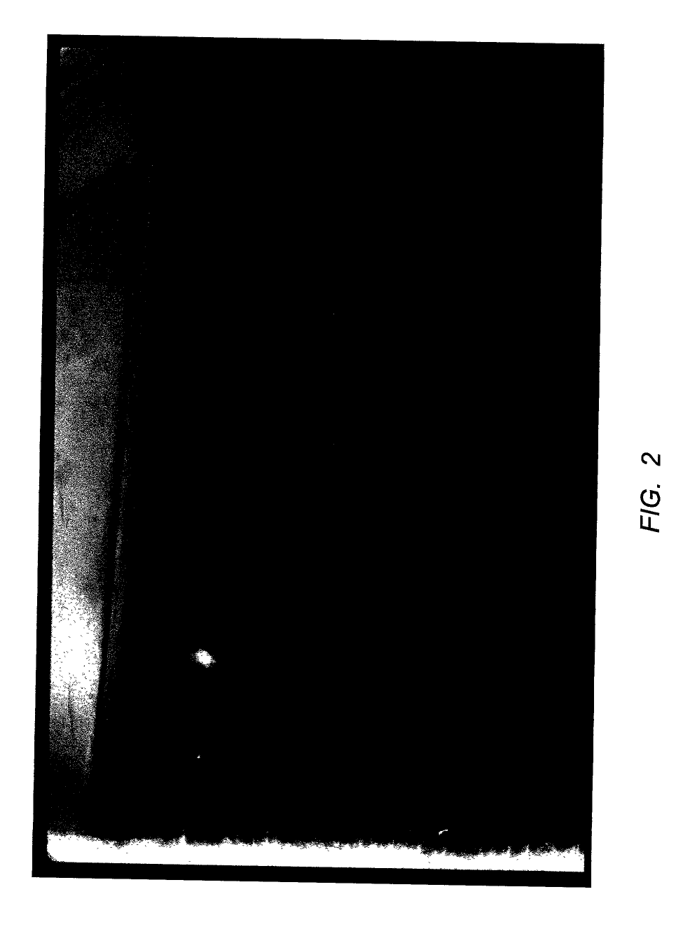

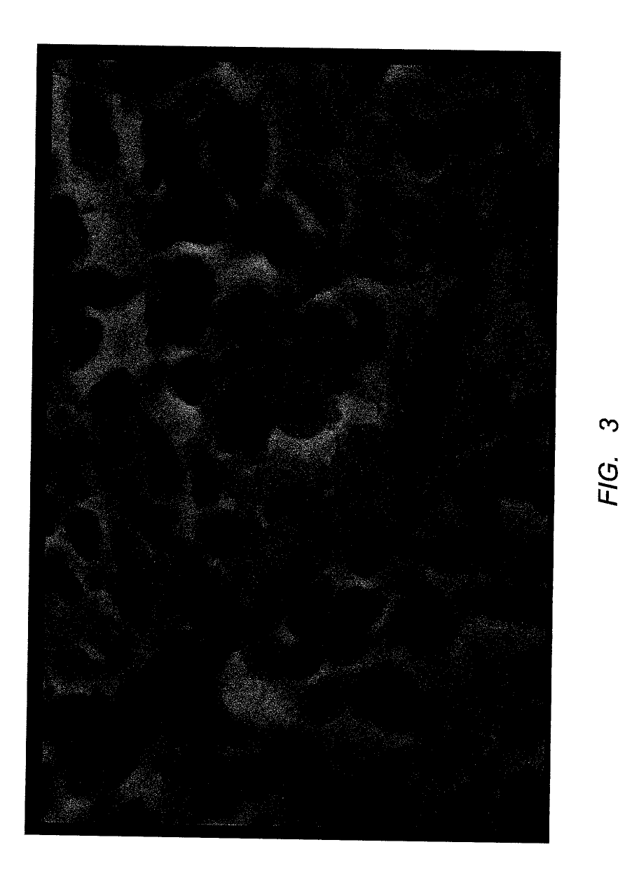

Similarly, diseased tissue samples co-incubated with such organism complexes in vitro may also indicate some disease-associated organisms. FIGS. 1 and 2 illustrate two vials in which such in vitro reaction tests were conducted. FIG. 2 shows a lymph node extracted from the same patient and cultured with penicillium fungus; filaments surrounding the cancer began to develop within days. This organism demonstrated affinity to the cancer and could not be washed off the surface easily; within days of co-culture, it could be recovered from the surface and from fragments deep within the mass. Note in figure one a lymph node biopsy in a patient with non-Hodgkins lymphoma, floating freely in a test tube containing several microorganisms of low cancer affinity. After 48 hours, incubation and three washes with sterile saline, no organisms could be recovered from the tumor mass.

Samples of the patient's blood or diseased tissue should also be tested in vitro against the potential disease-associated organisms. Any precipitation may indicate the presence of free-floating antigens associated with the disease, and may further indicate a potential association between such disease and the tested disease-associated organism. Also, in vitro testing of antibodies from the patient's immune system may indicate that some of the patient's own antibodies may be useful as disease-associated organisms against the targeted disease. Indeed, the antagonistic activity of such antibodies may be enhanced in vitro, as discussed in greater detail herein, and subsequently re-inoculated into the patient for transferring such enhanced antagonistic activity to the patient's immune system.

Unusually low titers from the in vitro tests of potential disease-associated organisms with the patient's blood or diseased tissue may indicate that the patient's immune system has been suppressed with regard to a particular potential disease-associated organism. If so, the organism should be considered as a possible synergistic or causative organism. Such indication would be confirmed with significant antigen titers in serum. Conversely, high titers of antibody (or dermal reactivity, when tested in vitro) may indicate previous exposure of the patient to the potential disease-associated organism, which may again be indicative of a possible disease-associated organism. Confirmation of this possibility would be given by the occurrence of significant antigen titers in serum.

In tests where there is a low antigen titer, a high reactivity may indicate the presence of a very useful tagging or immuno-stimulating organism. Titers of antibodies or antigens in response to any of the potential disease-associated organisms are indicative of the presence or susceptibility of the patient to another disease which is associated with such organisms. Thus, in vitro testing of components of the patient's body may be further useful as a method for monitoring the potential threat to the patient by a previously undiagnosed disease.

In vitro testing may be used still further to diagnose the presence of various diseases in a patient's body, which diseases can be subsequently treated by use of various identified disease-associated organisms including those which may be in an existing library of known disease-associated organisms. For example, various anti-bacterial antibodies which have been raised or developed for known diseases may be added to a patient's blood sample. Any precipitation will indicate the presence of a bacteria or its products in the patient's blood stream). The extent of the precipitation reaction can indicate the extent of the disease. Such in vitro testing may be used even further to monitor the status of a targeted disease in a patient.

Once the spectrum search is complete, and various tests have indicated various disease-associated organisms, it may be desirable to enhance the affinity of one or more of the disease-associated organisms toward the targeted disease. Such affinity enhancing may be accomplished through in vitro or in vivo techniques. It has been found, for example, that co-incubating organisms in alternating minimal and enriched media can be an effective method of stimulating various desirable organism characteristics, such as an increased affinity or antagonism between such co-incubated organisms.

In cases where no apparent attraction exists between a neutral organism and a targeted disease, such attraction may be trained or bred into the organism via co-incubating the neutral organism with cells of the targeted disease in a minimal or partially deprived media. It has been found that after the nutrition in the media is exhausted, the neutral organism will attempt to adapt to its surroundings and develop any attraction which may be needed for procuring nutrients which might be available to it by some activity toward the targeted disease. Such nutrients may be made available to the once neutral organism through ingestion of the disease, as in the case of the development of antagonistic activity in the organism, or the nutrients may be made available through the development of some type of synergistic relationship with the disease.

It has been found, for example, that some bacteriophages will emerge from various bacteria which have been partially ingested by another organism, and will then infect the organism that destroyed the original host bacteria. The attacking organism acquired the nutrients of the ingested bacteria, and the bacteria-phage acquired a new host. This particular characteristic of phages makes them especially powerful when used as therapeutic agents. When a phage that demonstrates anti-agonistic activity toward a targeted disease is used as a therapeutic agent, its nucleic acid may actually move into the body's lymphocytes, allowing the phage's behavior toward the disease to reside as part of the body's long-term immune memory. The body is then able to regenerate either the phage or activity in the lymphocytes similar to that of the phage whenever the body is subsequently exposed to the targeted disease.

After the disease-associated organism has demonstrated such an antagonistic or affinitive activity toward the disease in the minimal media, it should be transferred to an enriched media in which there mayor may not be cells of the disease, for stimulating growth in the now changed organism. It is preferred that such media enriched cultures are also stimulated by various mutant-precipitating factors in order to induce continued changes on the part of the disease-associated organism. After sufficient time for allowing the desirable growth by the disease-associated organism, various portions of it should be tested again for identifying the portions having the greatest affinity or attraction for the disease cells or diseased tissue. Such affinity testing may include those tests described earlier herein, or other tests. It is preferred that such portions having the greatest attraction be still further subjected to co-incubation with fresh cells of the disease in minimal media, for stimulating an even greater attraction toward the disease. It has been found that several iterations of this minimal-media-to-enriched-media cycle can induce a significant antagonism or affinity in a disease-associated organism toward a targeted disease.

Further, it has also been found that such bred-in attraction becomes a substantially permanent characteristic of the disease-associated organism. This method may be useful for both enhancing the degree of any antagonism or affinity which may already exist in a naturally antagonistic or affinitive organism, and for creating a disease-specific antagonism or affinity in cells of a selected organism, which cells have not heretofore demonstrated such antagonism or affinity toward a targeted disease. Also, the creation or enhancing of such antagonistic or affinitive activity toward a targeted disease may be accomplished with cells of the patient's own immune system, as will be described later, herein. A variation of affinity enhancing involves similar testing and raising techniques in vivo with animals not having natural immune systems, such as skid rats and nude mice. Such in vivo testing is especially useful when it is desirable that the ultimate therapeutic agents do not depend at all upon the patients immune system for overcoming the targeted disease.

It has also been found, regarding the purposeful increasing of disease-affinity or antagonism in a disease-associated organism, that a plurality of mutations may occur in the targeted disease, resulting from the disease's attempt to defend itself from the disease-associated organism. Such stimulated in vitro to in vivo mutating on the part of the disease has been found to be quite useful in preventing the occurrence of similar mutations in the patient, and in establishing an accurate basis upon which a prognosis regarding the targeted disease can be made. Further, the inventor has found that continuing the minimal-media-to-enriched-media cycle will provide a plurality of mutants to a disease-associated organism which may be useful as therapeutic agents against corresponding mutants of the targeted disease. When such agents are used in coordination with versions of the disease-associated organism having a strong attraction for current forms of the disease, it has been found that the targeted disease has great difficulty in developing a resistance to the disease-associated organism. In military terms, it is not dissimilar from placing mine fields and ambush teams along the potential paths of a retreating enemy.

It has also has been found that vaccines prepared from detrimental disease-associated organisms which do not have a great affinity toward a targeted disease, can be effective when administered via direct injection into diseased tissue. This type of administration may also be helpful for insuring that a patient's immune system is not able to block a particular therapeutic agent from contacting the disease. The therapeutic agents which have a low affinity toward a targeted disease may also be linked to other organisms which have been found to have a strong microbial-affinity to the targeted disease, as is addressed further, below.

The next step in the method of the present invention is to prepare a therapeutic agent or agents from the disease-associated organisms. It is preferred that each found and tested detrimental organism is crushed, lysed or otherwise fractioned via means which are known (e.g., physical lysis, chemical lysis, biological lysis, radiological lysis or the like) in order to reduce each organism to fractions which are sufficiently small such that when placed inside the patient a general systematic immune response against the antagonistic organism is prevented. For example, with human patients it is desirable to filter any non-human sera via means which are known or which have been described herein, for preventing any potential allergic reactions. It has been observed that when prepared as a vaccine, therapeutic agents made in accordance with the present invention and having no component thereof which is greater in size than 0.2 microns, will elicit only little, if any, systematic immune response. A further advantage beyond that of size which may be gained in not using entire disease-associated organisms in a vaccine, includes the ability to have a more controlled breakdown or die-off rate of the targeted disease, and the attendant occurrence of fewer side effects resulting from such breakdown.

Prior to full application of the therapeutic agents, it is preferred that they are tested in vivo for potency. Phage-lysed Staphylococcus albus, for example, has been found to be well tolerated when used as a therapeutic agent in doses of 2.5 cc every second or third day. Should the attending medical practitioner have experience with the particular targeted disease, he may wish to begin such testing with therapeutic agents of disease-associated organisms that experience has indicated precipitate strong antigenic responses. It is recommended that initial in vivo testing be conducted intradermally, in order to observe the cutaneous reaction to the vaccine. Initial dosage should be in the 0.1 cc range. This dosage may be increased by doubling daily until the size of the local reaction is six inches or greater. The patient's temperature will often rise to 103° F. and last a few hours.

Depending on the size and activity of the compounds, continuing administration of each therapeutic agent may be accomplished via a wide variety of administration means which are known. Decline of the targeted disease, such as tumor shrinkage, should be demonstrable in blood tests within a few days after the initial vaccination. Tumor shrinkage should be demonstrable on X-ray within two or three weeks. If such disease decline is not observed, the same treatment should be repeated with alternate therapeutic agents or vaccines from other of the cultured pathogens until such effect is observed. A more detailed explanation of what actions are available in accordance with the method of the present invention for overcoming a highly resistant disease, is provided below.

As discussed, therapeutic agents prepared in accordance with the method of the present invention may be introduced to a patient by any of a wide variety of administration means, such as those selected from the group consisting of topical administration, injection directly to diseased tissue, oral administration, rectal administration, intraurethral administration, intravesical administration, inhalation, intradermal administration, intrathecal administration, catheter administration, intravenous administration, intramuscular administration, subcutaneous administration, intravenous administration, and intradermal administration. Those skilled in the art will recognize other means of administration which may also be used in accordance with the present invention.

Toxicity studies have failed to demonstrate oral toxicity at 5 mg/kg in mice. Similar safety is demonstrated for agents prepared in accordance with the instant invention in subcutaneous, intramuscular, or intravenous application at 1 mg/kg. The intradermal method is preferred when dealing with lysed bacterial fragments, filtered extracts of bacteria or other organisms which have been biologically, chemically or physically lysed. When whole organisms are utilized as therapeutic agents, it is preferred that they are administered orally or applied extradermally. Oral administration may be in the form of enhanced foods and food supplements.

Extradermal administration may be in the form of a cosmetic. For example, the inventor has combined in one cosmetic solution an organism which is antagonistic toward human dead skin cells, and fragments of another organism which has rejuvenating characteristics. The antagonistic organism works as an exfoliant, taking off layers of dead and hardened skin which many exfoliants cannot touch. Simultaneously, the organism gives a rejuvenating effect to the living skin cells below the outer layer of dead skin. Since the exfoliant in this cosmetic is a living organism, its effect upon the skin tends to be much longer lasting than other skin conditioners.

### EXAMPLE 2

Another method for producing therapeutic agents according to the present invention involves the identification and use of either those organisms which demonstrate a beneficial relationship toward a targeted disease (i.e., causative or synergistic organisms) or those organisms whose activity toward a targeted disease is substantially neutral (i.e., neutral or infective organisms). Like most antagonistic and nemesis organisms, beneficial and neutral organisms usually demonstrate a microbial affinity toward cells of a targeted disease or diseased tissue. However, some beneficial and neutral organisms may demonstrate a type of synergism toward a targeted disease which is remote from the disease, and which synergism may not be demonstrated by a microbial attraction between cells of the beneficial or neutral organisms and cells of the disease or diseased tissue. Rather, the remote synergism is generally demonstrated in that such beneficial and neutral organisms live in an apparent harmony with the patient's body, even though such beneficial and neutral organisms may be foreign to the patient and may normally elicit an immunological response in such a patient-specie host.

As shown in FIG. 3 a Russel body is marked by arrow. Such bodies have often been noted in cancer biopsies. Early researchers thought them to indicate blastomycetes. The inventor believes them to be yeast or fungal etiology.

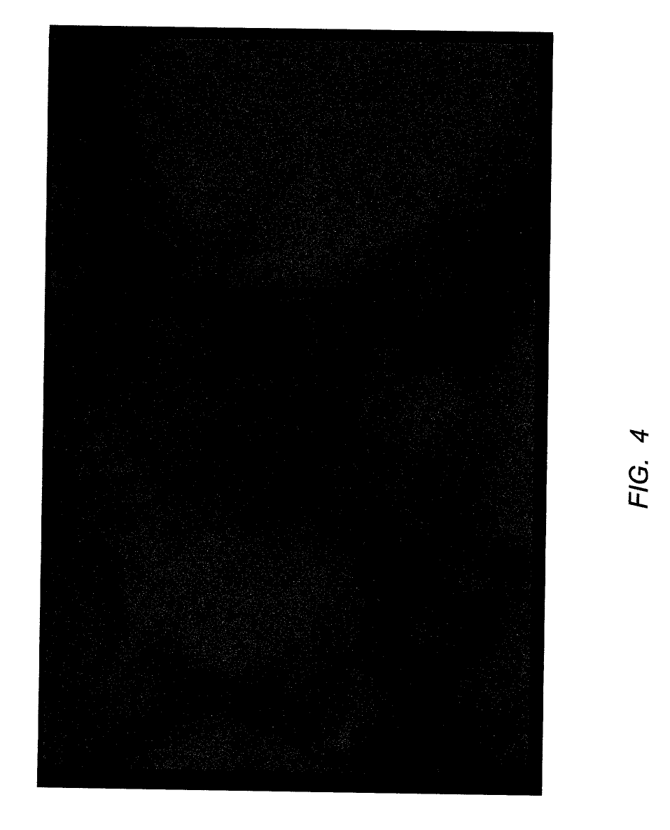

FIG. 4 shows several coccoid forms forming a nest in a biopsy of breast cancer. In both FIGS. 3 and 4, note the total lack of any immune response. Although such an observation might be expected in AIDS patients as a result of dealing with an exhausted or otherwise suppressed immune system, substantially similar observations have been made by the inventor in a wide variety of other diseases. It is believed that such harmony between this living foreign matter and the patient's body is due to a shared immune barrier between the beneficial or neutral organisms and the disease. Furthermore, the inventor has discovered, that breaching such barrier by attacking the beneficial or neutral organisms can also result in destruction to a targeted disease. Accordingly, the spectrum search for potential disease-associated beneficial and neutral organisms should include looking throughout the patient's body, or the body of another patient-specie host infected with the same or a similar disease, for any organism which is not naturally associated with such a host.

A spectrum search for beneficial and neutral organisms should be conducted simultaneously with the spectrum search for detrimental organisms, and should also include an epidemiological search. However, the epidemiological search for beneficial and neutral organisms should focus on agents which thrive in geographical areas having a high incidence of the targeted disease. Statistical indicators which point to higher correlations of various agents and a targeted disease should also be noted. An in vivo search should follow, beginning in the patient's body, and extending to the bodies of other patient-specie hosts having the same or a similar disease. As noted, above, a blood sample or biopsy of the patient's diseased tissue may reveal a beneficial or neutral organism which is attracted to, or otherwise living in a synergistic relationship with, disease cells or diseased bodily tissue. Because an effective attack made against beneficial or neutral organisms may also be effective in defeating anti-immune factors which may be present in the targeted disease, special attention should be given to such organisms found in a patient's body which are not being attacked by the immune system. In looking for such beneficial or neutral organisms, it should be noted, that some stains will not readily differentiate particular bacteria from diseased tissue, while other stains have no difficulty in providing a sufficient visual difference for such differentiation.

Beneficial or neutral potential disease-associated organisms usually can be found within the protective encapsulation of diseased tissue such as a tumor. As noted earlier, such beneficial or neutral potential disease-associated organisms often can be found in locations of a patient's body which are remote from the targeted disease.

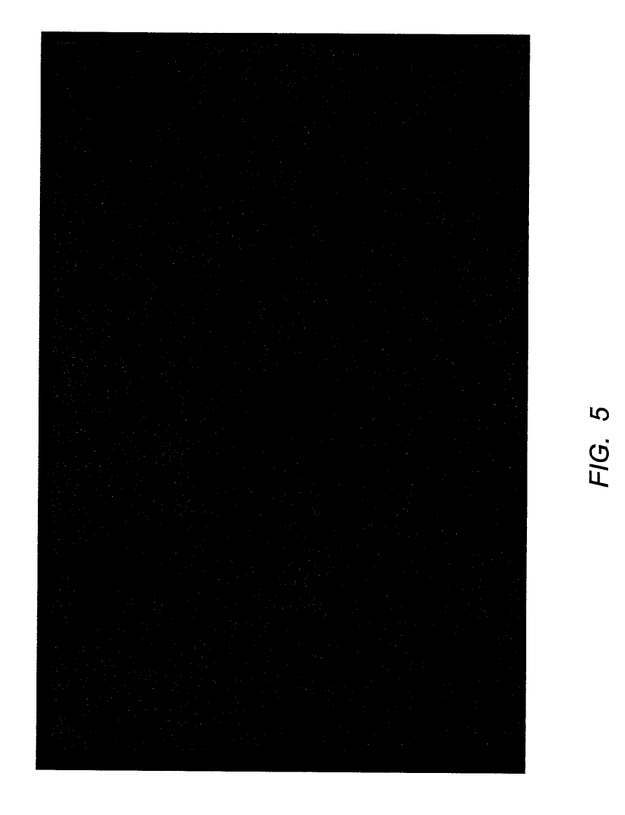

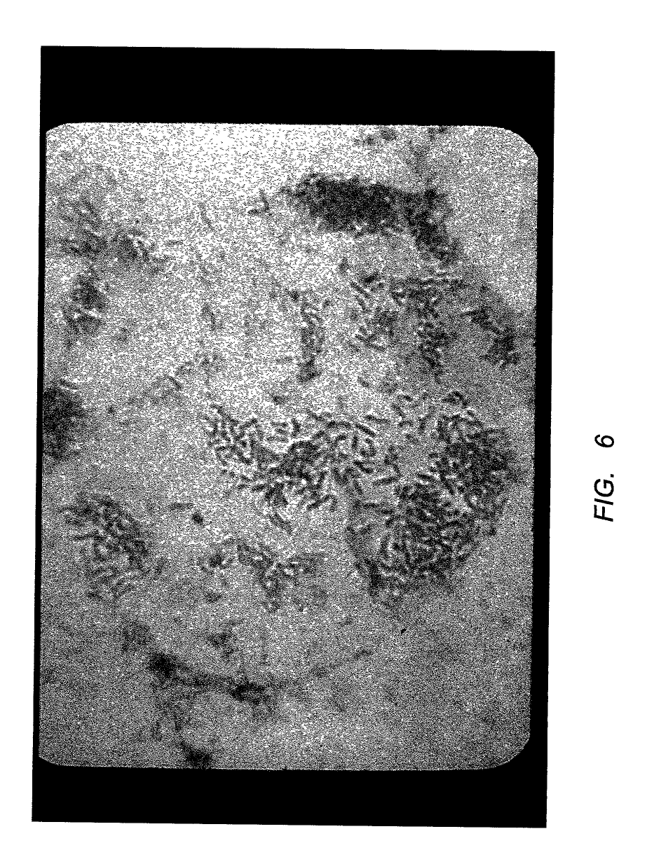

Still further, the inventor has found that such synergistic harmony between a targeted disease and a beneficial or neutral organism which is not eliciting an immunological response, may also exist outside of physically apparent boundaries. For example, it has been found that the bacteria, staphylococcus albus will grow readily from what appears to be a “clean” tissue sample of a patient having carcinoma. When the inventor first noticed such an occurrence, the tissue sample had been fractioned and filtered through a 0.2 micron filter, which is sufficiently small to prevent the transfer of any staphylococcus albus cells. For example, FIG. 5 shows a sample of mixed coccal culture which was grown from such a “clean” blood culture of an AIDS patient. FIG. 6 shows a culture of tubercule bacilli grown from a “clean” skin sample of a scleroderma patient. Repeated occurrences of this phenomenon have led the inventor to believe that various diseases may be transmitted, or even may lie dormant, in non-cellular forms, such as in the form of cell-wall deficient bacteria or of genetic information in various phages or plasmids, and that such forms will ultimately materialize as cellular structure once the right conditions exist. Accordingly, patient blood samples and biopsies of diseased tissue should be cultured by culture techniques such as those which are described elsewhere herein in order to isolate any potential disease-associated organisms which may not be readily identifiable. When the targeted disease is of the type which precipitates the production of blocking antibodies for coating diseased cells in order to protect them from the remainder of a host's immune system, the search for such beneficial or neutral organisms should include attempts to isolate the blocking antibodies using pH precipitation techniques. As described further herein, “disease-friendly” antibodies of a host's immune system which are attracted to a targeted disease may be used even to carry antagonistic agents to cells of the targeted disease.

Once such beneficial or neutral potential disease-associated organisms have been found, in vitro or in vivo testing of the type described earlier should be performed to verify that the organisms are indeed disease-associated organisms of the targeted disease, and to further verify or determine the type and level of the affinity or synergism the organisms may demonstrate toward the targeted disease. Therapeutic agents using the beneficial or neutral disease-associated organisms may then be prepared and utilized.

A general therapeutic approach in using beneficial or neutral disease-associated organisms involves identifying other disease-associated agents which demonstrate antagonistic or nemesis activity toward such beneficial or neutral disease-associated organisms, including the extracts, by-products or modifications of the beneficial or neutral organisms. The disease-associated agents therapeutic agents which are useful for attacking the beneficial or neutral agents in order to break down the targeted disease's anti-immune factors which may be shared with the beneficial or neutral disease-associated organisms. It has been found that such other therapeutic agents can often be used to mount an effective attack against the beneficial or neutral agents, and that such an attack can also result in destruction to the targeted disease.

The next step in the method is to identify disease-associated agents which are antagonistic toward the beneficial or neutral disease-associated organisms. The disease-associated agents can normally be located through epidemiological searches, in vitro testing or in vivo raising in different-specie hosts. Once such agents are found, the next step is to determine the type and level of activity such agents demonstrate toward the beneficial or neutral organisms. In making this determination, it is preferred to use substantially the same type tests as described in the first embodiment of the method of the present invention. Any affinity or antagonism a disease-associated organism may demonstrate toward a beneficial or neutral organism may be increased by the incubation of the disease-associated organism in alternating minimal and enriched media with cells of the beneficial or neutral organism, or cells of other organisms it may be desirable to increase such affinity or antagonism toward. For example, these may include disease-associated organisms that are useful as tagging agents for the disease due to their existing high affinity toward the disease. If, for example, a spectrum search results in the identification of a disease-associated organism which is only slightly antagonistic toward a beneficial organism found living with impunity inside a large tumor mass, it may be desirable to increase the microbial affinity and antagonism between the disease-associated organism and the beneficial organism. This enhances the ability of the disease-associated organism to penetrate the outer walls of the tumor for attacking the beneficial organism, thereby causing collateral destruction to the tumor mass. As an alternative to petri dishes and in vitro reaction test vials, in vivo testing with animals not having an immune systems should also be considered as a viable means to further accomplish such affinity or antagonism enhancing.

Once the disease-associated organisms and other agents have been isolated and any desirable level of affinity or antagonism between such agents and other organisms has been attained, the next step is to prepare a therapeutic agent or agents from each of the disease-associated organisms, using methods substantially similar to those described in the first embodiment. As with the first embodiment, it is preferred that each vaccine is prepared from organism fractions rather than from whole organisms. This reduces the possibility of any systematic immune response against the vaccine, even though there are times when the use of whole organisms in various suitable applications may be desirable.

Administration of the therapeutic agents may be accomplished by means which are substantially similar to those of the first embodiment. For protocols involving the administration of both therapeutic agents made from beneficial or neutral organisms and therapeutic agents which are antagonistic to such beneficial or neutral agents, it is preferred that the patient is administered the beneficial or neutral agents in a sufficient amount of time before the administration of the antagonistic agents. This will allow a sufficient synergism to develop between the beneficial or neutral therapeutic agents and the targeted disease for allowing attacks against the beneficial or neutral agents by the antagonistic agents to be most effective in producing collateral damage to the disease. A sufficient amount of time may be anywhere from about several hours to about several days, depending on the level of synergism which is desired. Once the antagonistic organisms are effective in breaking through any immune factor which any beneficial or neutral organisms may have shared with a targeted disease, it has been found that the immune system of the body will also be stimulated to help fight both the beneficial or neutral organisms and the targeted disease.

### EXAMPLE 3

A third embodiment of the method for producing therapeutic agents according to the present invention involves using therapeutic agents made from disease-associated organisms having a microbial affinity toward the disease cells or diseased tissue as tagging agents. Such affinitive disease-associated agents, or disease-affinitive agents, include any of the disease associated organisms already discussed (i.e., causative organisms, synergistic organisms, neutral organisms, infective organisms, antagonistic organisms and nemesis organisms), including whole, part or extracts thereof. Any of these organisms, extracts or modifications thereof, which demonstrate a microbial affinity or attraction for the targeted disease or diseased tissue may be useful as tagging agents. Such tagging agents may be useful for making disease affected areas of a patient's body more visible to the patient's own immune system or to another therapeutic agent which is antagonistic toward the tagging agent, the tagged material or by-products of the tagging agents or the tagged material. Similar to organisms which have developed a symbiotic relationship with a targeted disease, it has been found that attacks which are instigated on such tagging agents by either the patient's immune system or other antagonistic therapeutic agents, can also result in destruction of the tagged disease or diseased tissue. Also, tagging agents may be useful for carrying other agents (such as those which are antagonistic toward the targeted disease but which lack a microbial affinity toward the disease. Other tagging agents may include those which have been used without great specificity in conventional therapy, such as chemotherapeutic agents or radioactive isotopes) or intracellular information directly to disease cells.

The initial steps of this embodiment include identifying or raising disease-affinitive organisms by techniques substantially similar to those used in identifying and raising detrimental and beneficial organisms. The steps of identifying and raising disease-associated organisms should include looking for and raising both organisms which have a high affinity for diseased tissue and organisms which have a high affinity for healthy cells, via minimal and enriched media culturing techniques, substantially similar to those techniques earlier described herein. In raising organisms having a high affinity toward healthy cells via this manner, organisms which are antagonistic to these healthy-cell-affinitive organisms are subsequently raised either in vitro or in vivo. Once raised, the anti-healthy-cell-affinitive organisms may be applied, or “washed,” against organisms having an affinity toward a targeted disease, for insuring that any affinity between the diseased-cell-affinitive organisms and healthy cells is eliminated prior to introduction of the diseased-cell-affinitive organisms into the patient. It is preferred that the diseased-cell-affinitive organisms, extracts or modifications thereof, are washed in this manner repeatedly against the anti-healthy-cell-affinitive organisms, extracts or modifications thereof, for insuring that any cells of the diseased-cell-affinitive organisms are eliminated prior to introduction of the diseased-cell-affinitive organisms into the patient. Furthermore, the diseased-cell-affinitive organisms, extracts or modifications thereof, may be similarly washed repeatedly against disease cells in order to extract from the sera only those cells which have the greatest affinity toward disease cells. The disease cells may then be removed from such cells having the greatest affinity toward the disease via chemical, physical or biological separation methods which leave the diseased-cell-affinitive organisms, extracts or modifications thereof, intact and substantially ready for introduction into the patient. Such disease-cell-affinitive organisms, extracts or modifications thereof, raised and washed in this manner will have both a high affinity toward the targeted disease and a high specificity regarding the type of cells to which they are attracted. Indeed, it has been the inventor's experience that patients treated with such diseased-cell-affinitive organisms, extracts or modifications thereof, have developed only minimal, if any, side effects. Also, it may be desirable to raise the affinity of such affinitive organisms toward the targeted disease via the in vitro and in vivo methods already discussed. As well, oncolysates of the disease (which are discussed in greater detail further herein) may be used to enhance the affinity of a tagging agent for a targeted disease, via allowing fragments of the targeted disease to be attached to the tagging agents in vitro before the tagging agents are introduced to the body.

Since a main purpose of tagging agents is to make the targeted disease more visible to the patient's immune system, or to other therapeutic agents which are antagonistic toward the tagging agents or tagging-agent-disease complex, the next step is to raise an immunity in the patient against such affinitive organisms or against a combination of such affinitive organisms and tissue of the targeted disease, including any of their extracts or by-products. Raising such immunity in the patient may include stimulating the production of various antibodies to the tagging agents in the patient's immune system, or, as in cases in which a patient's immune system is already severely taxed, adding to the patient therapeutic agents made from organisms which are antagonistic toward the tagging agents or the tagging agent and targeted disease complex. Stimulating the production of antibodies may include the conventional means of vaccinating the patient with dead or inactivated forms of the tagging agents prior to inoculating the patient with active forms thereof. in vitro or in vivo testing may reveal, however, that such vaccination is not necessary; or, physician judgment may determine that stimulation of the production of patient antibodies against the tagging agents may not be warranted at this time. Organisms or agents which are antagonistic toward the selected tagging agents or complexes of the tagging agents and diseased tissue, may be identified and tested via methods substantially similar to those already described herein. Enhancing the antagonism of such antagonistic agents toward the tagging agents or tagged diseased complex should also be explored, via methods described earlier herein.

Once the disease-associated organisms and other tagging and anti-tagging agents have been isolated and any desirable level of affinity or antagonism between such agents and other organisms has been attained, the next step is to prepare a therapeutic agent or agents from each of the disease-associated organisms, using methods substantially similar to those described earlier herein. Similar to the first embodiment, it is preferred that each vaccine is prepared from organism fractions rather than from whole organisms, in order to reduce the possibility of any systematic immune response against the vaccine; although there are times when the use of whole organisms in various suitable applications may be desirable.

Administration of the therapeutic agents may be accomplished by means which are substantially similar to those of the first embodiment. It is preferred that the patient is administered the tagging agents in a sufficient amount of time before the administration of the antagonistic agents, in order that a sufficient synergism be developed between such tagging agents and the targeted disease for allowing attacks against the tagging agents or the tagged disease complex by the antagonistic agents to be most effective in producing collateral damage to the disease. The amount of time may be anywhere from about several hours to about several days, depending on the level of synergism which is desired. Rather than using general inoculation techniques as earlier described herein, it may be desirable to inject such tagging agents directly into disease tissue, or such anti-tagging agents directly into the tagged disease complex, if, for example, it is found that the patient's immune system will yield an extremely high antibody response to the selected tagging agents or anti-tagging agents. Such direct injection would prevent the patient's immune system from blocking such affinitive agents from the diseased tissue. For tagging purposes, the amount of the tagging-agent which is used may be sufficiently small (e.g., a tenth or even a hundredth of the normal dose of an antagonistic agent) so as to avoid stimulating an immune response against it before the targeted disease is tagged. Once the disease is tagged, a higher amount or concentration of the tagging-agent may then be applied to the patient, with or without adjutants such as silica or Friend's adjuvant, to stimulate the body's immune system against the tagging agent and the tagged disease.

With regard to the use of using tagging agents for carrying other agents or intracellular information to disease cells or diseased tissue, it has been found that smaller antagonistic organisms, such as viruses and phages, may be carried readily to targeted disease cells by combining them with larger affinitive disease-associated organisms, such as bacteria and fungi. Such combining may be accomplished by culturing in diet restricted media the organisms which are antagonistic to the targeted disease (or extracts or modifications of these antagonistic organisms) with organisms that are attracted to the targeted disease. Elements of the smaller antagonistic organisms may be ingested by the larger organisms, and thereby be available for transport by such larger organisms directly to disease cells. If such carried antagonistic organisms are phages, the phage will often infect the larger affinitive disease-associated organism while in vitro, and be carried to the targeted disease by the larger disease-associated organism upon the application of such organism to the patient. When the affinitive disease-associated organism makes membranous contact with the disease cell, the phage should be transfused from the disease-associated organism, through the membrane of the diseased cell and into the cytoplasm of the targeted cell. It is believed, further, that tagging agents may be useful for carrying other specific nuclear information to disease cells, such as RNA or DNA coding which directs the cells to stop reproducing, to self-destruct, or the like.

### EXAMPLE 4

### Chemotherapy and Antibiotic Therapy

A fourth embodiment of the method for producing therapeutic agents according to the present invention involves chemotherapeutic agents which have been used therapeutically by the inventor with some positive results against a targeted disease. A common problem associated with known chemotherapeutic agents is the non-specificity of the agent.

The specificity of such chemotherapeutic agents can be enhanced by incorporation in their application of the use of affinitive organisms which have been identified as having a high affinity for the targeted disease. Such affinitive organisms include disease-associated organisms selected from the group consisting of causative organisms, synergistic organisms, neutral organisms, infective organisms, antagonistic organisms, and nemesis organisms. Instead of vaccinating a patient directly with a chemotherapeutic agent, such agent is rather applied in vitro to an affinitive organism, thereby tagging the affinitive organism with the chemotherapeutic agent. A vaccine of the tagged affinitive organism is then prepared in accordance with the method of the present invention and administered to the patient. The affinitive organism, extracts or modifications thereof, used in the vaccine will then carry the chemotherapeutic agent directly to cells of the targeted disease. Should it be desirable to further limit the effect of such chemotherapeutic agent on the patient's body, organisms which are antagonistic to the chemotherapeutic agent may be raised for preparing vaccines in accordance with the method of the present invention, which vaccines may be used to shorten the life of the chemotherapeutic agent inside the patient's body.

### EXAMPLE 5

A fifth embodiment of the method for creating therapeutic agents according to the present invention involves in vitro phage destruction of antagonistic organisms, beneficial or neutral organisms, cells of the disease, diseased tissue or any combination thereof, for yielding an oncolysate which may be useful as a therapeutic agent against the targeted disease. It has been found that denaturing of such antagonistic organisms, beneficial or neutral organisms, cells of the disease, diseased tissue or any combination thereof will yield oncolysates that have a high affinity toward cells of the targeted disease. Such phage destruction or denaturing can be stimulated via a variety of means, including, but not limited to, the following:

1) Allowing each isolated organism, disease cell or diseased tissue to grow in vitro in limited media until a phage arises spontaneously out of the culture.

2) Allowing each isolated organism, disease cell or diseased tissue to grow in vitro and inducing a stress upon the culture for precipitating a phage. Such stress may be administered via any of a wide variety of means, including physical, chemical, thermal, biological, ultraviolet light bombardment, pH stimulation, or the like.

3) Allowing a combination of targeted disease cells and an organism which demonstrates antagonistic activity toward these cells to grow in vitro until an oncolysate is formed as the targeted disease cells are consumed.

4) Allowing cells of the targeted disease to grow in vitro until the nutrition of the media is outgrown and natural degeneration of the disease cells occurs.

Each type of phage or lance fragment resulting from the above noted procedures should then be tested in vitro for its response to the targeted disease. The response may be further enhanced by methods discussed earlier herein. The resulting disease-associated agents should then be prepared as a therapeutic agent for administration to the patient in a manner similar to that of the first and second embodiments. It has been found that presentation of such therapeutic agents made of products of phages and other oncolysates can stimulate an effective immunological function and other therapeutic response concerning the targeted disease.

### EXAMPLE 6

A sixth embodiment of the method for producing therapeutic agents according to the present invention, concerns incorporating the use of erythrocytes, or red blood cells, into the human immunological system. It has long been assumed that the red blood cell has only a minor role, if any, in the body's immune system. This has perplexed the inventor, since the red blood cell is the most abundant cell in the human body. Because the mature red blood cell has a very flexible structure with no nucleus and appears to be little more than a highly resilient empty vessel for carrying water and hemoglobin throughout the circulatory system, the red blood cell appears fully capable of responding to, and even expressing, a wide variety of chemical, physical or genetic information, such as that carried by certain microbial extracts.

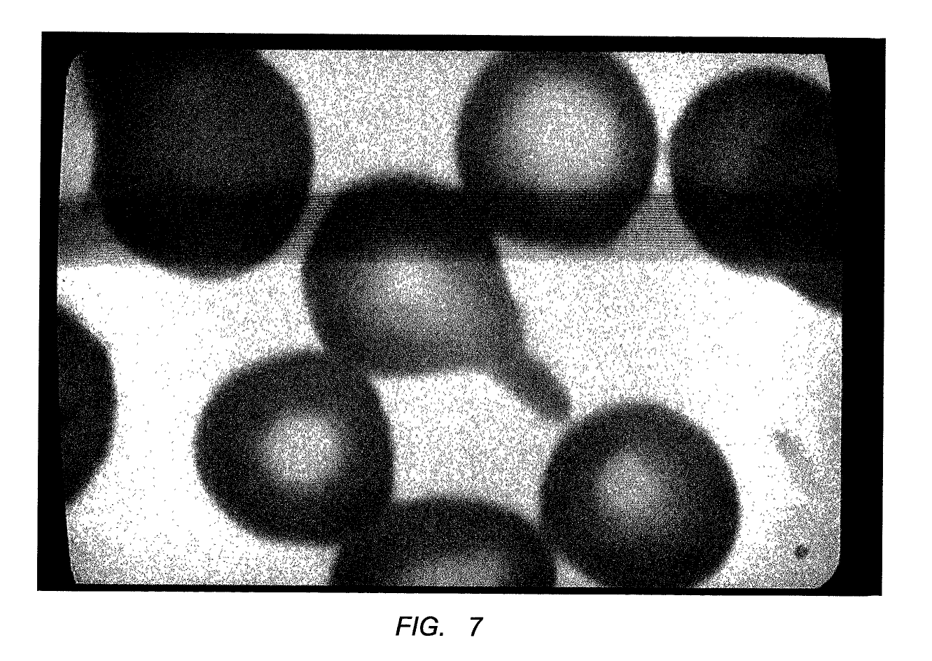

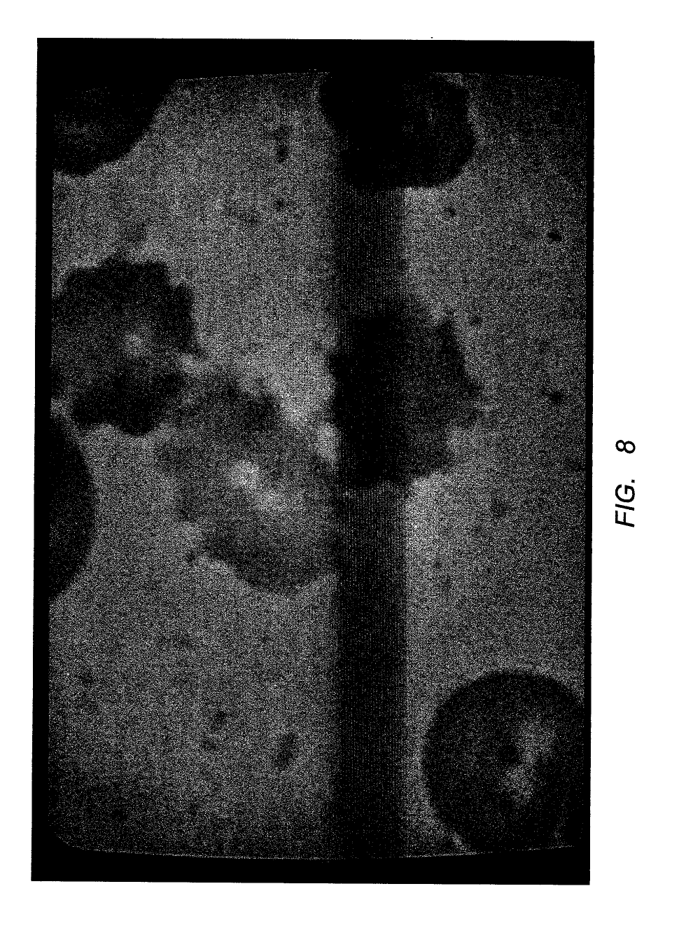

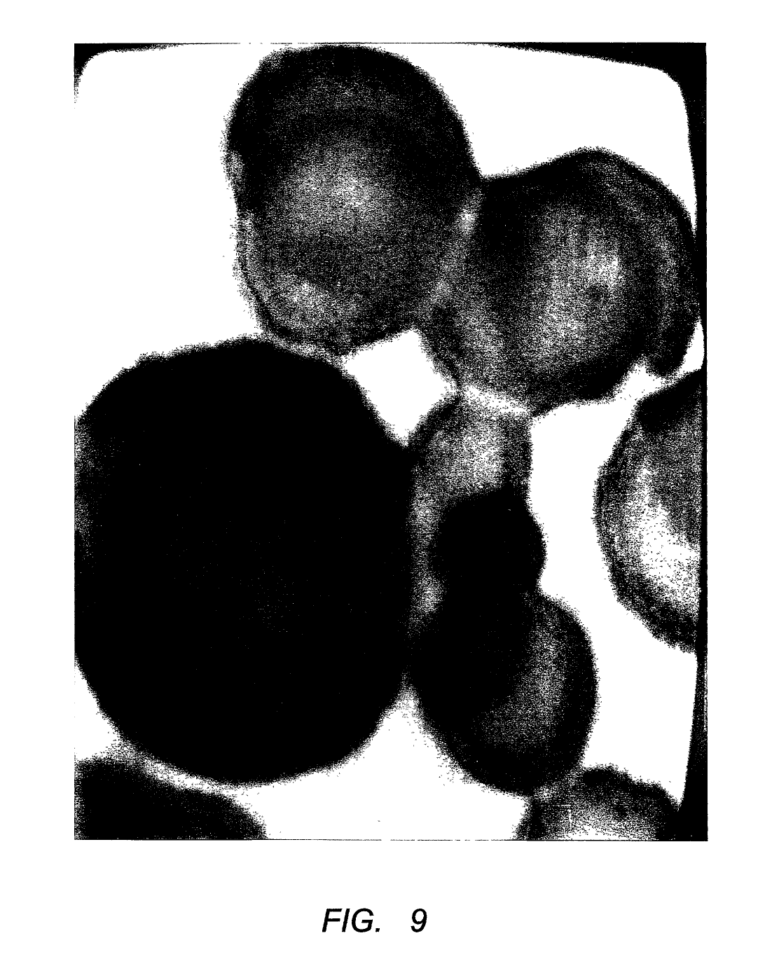

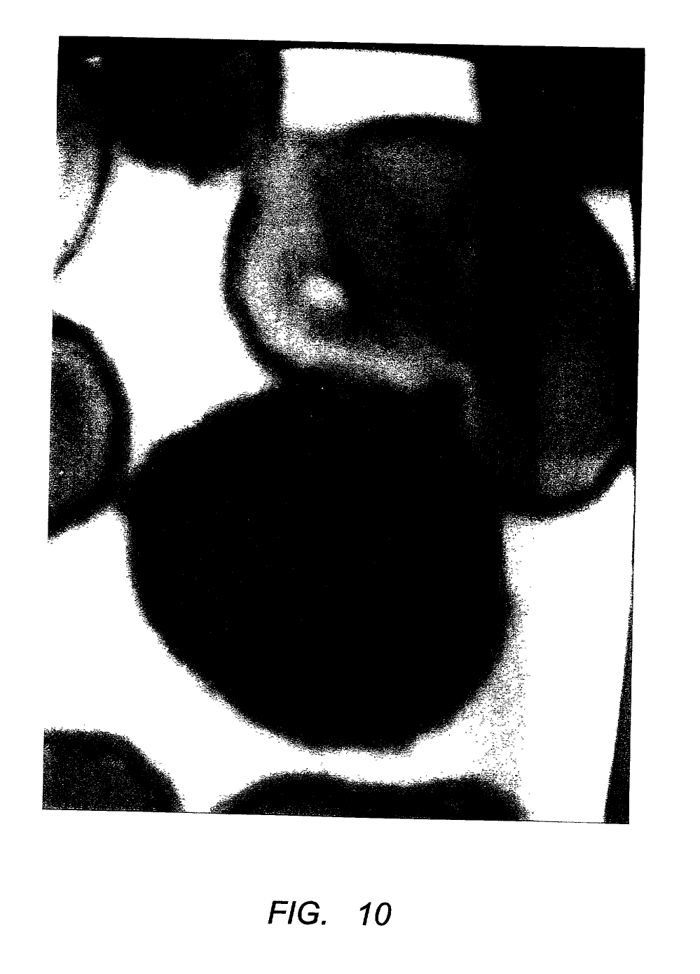

Indeed, it has been found that certain microbial extracts are capable of producing morphological changes in the appearance of red blood cells which appear to have a therapeutic effect with regard to a targeted disease. Such morphological changes are illustrated in FIG. 7, in which are shown red blood cells with tentacles formed in the membrane, and FIG. 8, in which are shown spicules in the membrane of several red blood cells, two of which cells have also taken on a ring-like form with an apparent hole in the center. Such morphological changes as those seen in FIG. 9 occurred apart from the presence of any such acidity, and were only temporary, resolving within a period of between a few hours and a few days. Although it has been known that red blood cells react to a high level of acidity by swelling into spiked balls (called echinocytes), similar transformation apart from such acidity has not been heretofore observed. The inventor believes that such changes may be functional, such as for example assisting in the movement of the red blood cells or assisting in the perforation of cancer cell membrane. FIGS. 9 and 10 show two groups of red blood cells in which inclusions have formed in their cytoplasm. Inclusions appear to have been extracted from attached leukemia cells.

An additional phenomenon which has accompanied such red blood cell changes as those noted above, is illustrated in FIGS. 9 and 10, in which is seen cell membrane fusion between adjoining red blood cells and between red blood cells and adjoining leukemia cells. Such points of contact between red blood cells and leukemia cells demonstrate areas of cellular fusion whereat the membrane boundary appears to dissolve, and cytoplasmic and nucleic contents of the leukemia cells appear to empty into the red blood cells. (See FIGS. 9 and 10). These red blood cell changes and activity in these several microphotographs were observed after application of vaccine created from the feline panleucopenia virus. Substantially similar morphological changes and anti-Leukemia cell activity has been observed after application of vaccine created from canine distemper virus. Both of these viruses were identified as disease-associated organisms regarding leukemia and subsequently prepared as vaccines by the inventor, according to the method of the present invention.

During in vitro testing, complete destruction of a sample of leukemia cells in a patient's blood sample was accomplished within a span of between two and three hours, without any pejorative effect whatsoever on the patient's normal cells. The results of subsequent treatment have included marked improvement and complete remission of the leukemia. Application of the therapeutic agent (i.e., the vaccine) in each case was sufficient to raise an effective immunological response in the patient, which response included the noted morphological changes and anti-Leukemia cell activity of the red blood cells.

It appears that information which caused such red blood cell activity was either transferred to the red blood cells by the applied therapeutic agents, or was awakened from a genetic memory within the red blood cells via administration of said agents. Such results indicate that the red blood cell may be actively manipulated into functioning as a member of the immune system via genetic programming, even such programming as that involving the transfer of genetic information between cellular materials which are in vivo. As earlier noted, it has been observed that a high number of cases of spontaneous remission have been reported which follow an acute malarial infection, an infection which could feasibly do more to the red blood cell than merely parasite it. It is possible that genetic information from the malarial infection, similar to that transmitted by the feline panleucopenia and canine distemper viruses, can transform red blood cells into an active arm of the immune system. Similar vaccines have shown dramatic preliminary results against HIV in vitro as well as in vivo testing even where no living organisms were used. Even dead extracts may cause therapeutic effects by a process of insertion, reactivation and expression as well as possible direct or indirect interference. If living or dead microorganism body or fragment may enter the disease target cell, they may interfere directly with the disease organism's ability to enter and/or multiply in the target cell by competition for binding sites, enzymes, co-factors involved in metabolism and replication of host or disease etc. Other host-related changes may also be induced which may also be therapeutic. Dead microbial extracts may, also be inserted or incorporated into the disease organism and/or diseased cell and serve as a direct marker antigenically or otherwise. Incorporation of microorganism fragments including nucleic acid fragments into the disease may give it characteristics of the organism used for therapy. Successful insertion of living microorganism into disease locale may bring about cross exchange of characteristics and properties or result in direct interference of one with the other. There is a risk of conferring undesirable capacity or resistance on the disease; this is reduced by the use of dead microbial extracts in therapy. Dead extracts may be reactivated to living form where microbial repair and multiplication apparatus occurs; this can often be found in the disease-infected cell. Reactivation of inanimate material and subsequent expression at the site of disease may provide for effective tagging and interference. Application of an antiserum to the reactivated micro-organism can eliminate both it and the diseased cell in a cycle that may be repeated.

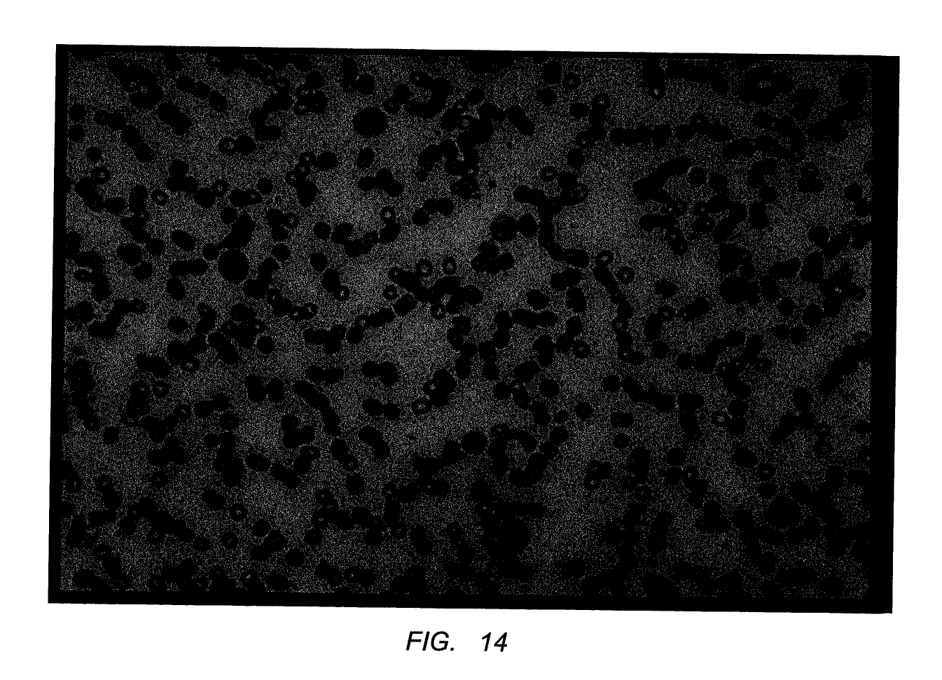

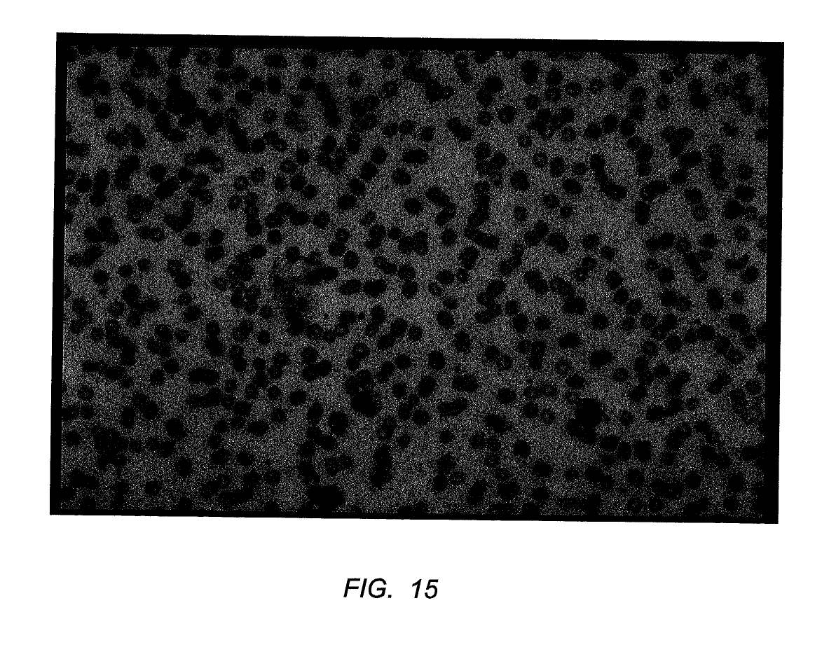

FIG. 14 is a blood sample plate from a 59 year old female having chronic lymphocytic leukemia. The dark blots are leukemia cells. FIG. 15 is a blood sample from the same patient taken within two hours (not a misprint) of the FIG. 14, during which time the patient was administered vaccines prepared in accordance with the method of the present invention. The white blood cell count has more than halved. A complete remission further occurred in this patient within several weeks. It is believed that the therapeutic agents made in accordance with the method of the present invention which precipitated these results, also changed many of the cancer cells into normal cells, resulting in the persistence of a higher red blood cell count until such time as the transformed cells had lived out the life-span of a normal cell, which is why the complete remission did not occur sooner.

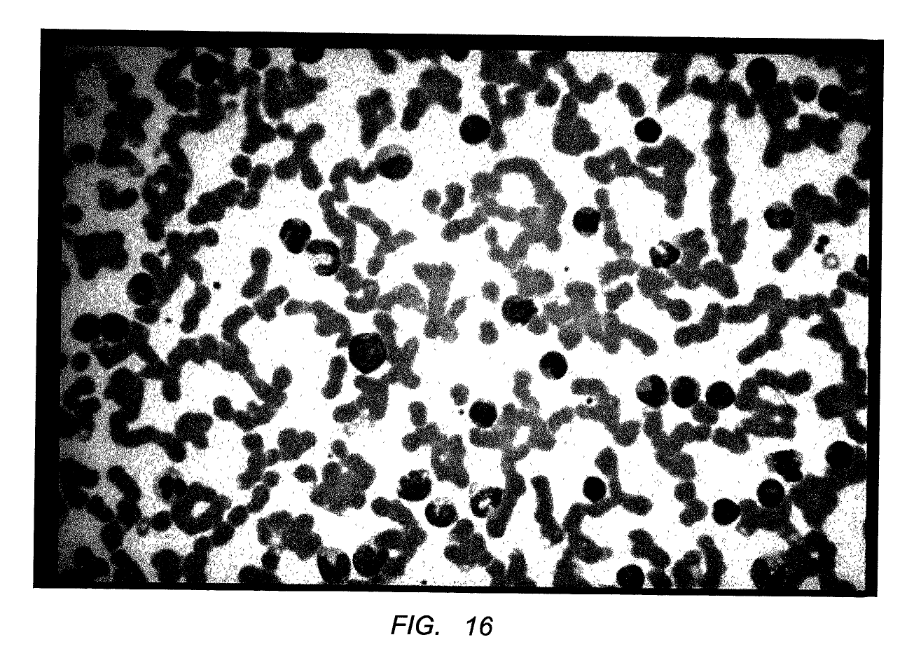

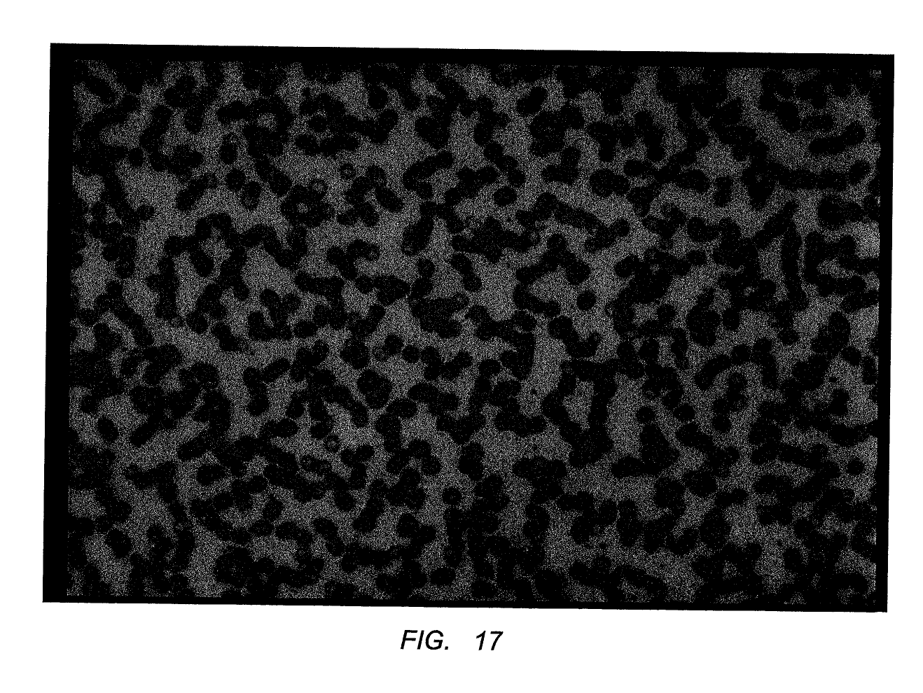

FIG. 16 is a blood sample plate from a 24 year old male having granulocytic leukemia. FIG. 17 is a blood sample from the same patient taken 7 days later, during which time the patient was treated with vaccines prepared in accordance with the method of the present invention.

The latter plate reveals a complete remission of the disease. Such a rapid and drastic reduction in the number of white blood cells and cancer cells in these two case histories cannot be explained apart from red blood cell activity, since such reduction had to involve cells of a number which surpassed that of the white blood cells and leukemia cells.

### EXAMPLE 7

A seventh embodiment of the method for creating therapeutic agents according to the present invention involves identifying the genesis of other chemotherapeutic agents which have been used with some positive results against a targeted disease. Although such agents may have been prepared using conventional time-consuming and expensive drug development techniques, an understanding of how said agents were prepared will point most often to an original disease-associated organism from which a particular chemotherapeutic agent was derived. If such an original disease-associated organism can be identified, the original organism may be utilized to develop still other derivatives for therapeutic use against various modifications of a targeted disease that may develop as cells of said disease respond to use of the chemotherapeutic agent by evolving into forms which are resistant to said chemotherapeutic agent. An antagonism in the original organism from which the chemotherapeutic agent was derived may be either raised or enhanced via methods earlier described herein pertaining to the culturing of a targeted disease with a disease-associated organism in alternating minimal and enriched media. As mentioned earlier, the disease cells will attempt to mutate further during such culturing process, in order to prevent annihilation by the detrimental disease-associated organism. Unlike non-living therapeutic agents, the detrimental disease-associated organisms will likewise evolve during such process in order to retain an efficacy against the disease cells. Therapeutic agents using the evolved detrimental disease-associated organism, extracts or modifications thereof, may then be used effectively against the associated mutated strains of the targeted disease. Those skilled in the art will readily recognize that accomplishing such mutations in vitro is an economical and an effective method of preparing therapeutic agents having a continuing efficacy against potential in vivo mutations of a targeted disease.

Indeed, it was the inventor's recognition of the unique ability of microorganisms to adapt quickly to their environment which led him to many of the therapies and methods for creating therapeutic agents according to the present invention. As the inventor has observed, the speed at which disease-associated organisms can adapt to environmental changes, may be used to prepare a library of disease-associated organisms, extracts and modifications thereof, which can be useful for preventing a targeted disease from gaining a perpetuating resistance to therapeutic agents. The inventor has further discovered that the simultaneous, or near-simultaneous, use of such therapeutic agents which are effective against several mutations of a targeted disease, can be very effective in overcoming the disease.

### EXAMPLE 8

An eighth embodiment of the method for producing therapeutic agents according to the present invention involves the genetic transfer of characteristics which have been identified as desirable, from a microorganism to a patient. It is known that various bacteria, viruses, parasites and other simple organisms have capabilities beyond those which are currently available in the body of man. For example, some bacteria have been identified as having sufficient heat resistance for allowing the bacteria to thrive within boiling volcanic pools. Other bacteria have sufficient thermal resistance to allow them to live in arctic regions. Still other bacteria have shown a high resistance to radiation. Yet still other bacteria are capable of photosynthesis, or the ability to transform simple sunlight into oxygen and forms of energy. It may be further deduced that the extra-regenerative capabilities of the salamander can be ultimately identified and linked to either a micro-organism or another biological factor. It is the belief of the inventor that such characteristics can be transferred across specie via methods outlined in the present invention, and thereby create whole new families of therapeutic agents.

Indeed, it has already been observed that certain regenerative characteristics from microorganisms having an extra-regenerative capability, such as those organisms which are thermally resistant or those which are radio-resistant, can be transferred from one specie to another. For example, the inventor has prepared vaccines from various bacteria which demonstrate such an extra-regenerative capability and further which have a high resistance to radioactivity.

When these vaccines were administered to laboratory rats, the survival stamina of the rats, as demonstrated by their ability to stay afloat unsupported in a body of water, was increased from an average of 45 minutes to well over six hours. Such a vaccine was also administered to a human patient who had undergone extreme digoxin (from digitalis) poisoning, and exhibited extensive damage to the heart and other portions of the circulatory system. Within twenty four hours, the patient had undergone a complete recovery, and demonstrated no sign of damage or scarring in either the heart and or any other portion of the circulatory system. Two lasting side effects have further been exhibited in this patient. The first involves the patient's normal energy level. Prior to undergoing the single dose treatment of the radioduran vaccine, the patient exhibited average sleeping patterns and habits. For three years since the treatment, however, the patient has been able to stay awake continuously for periods of multiple days without any apparent loss of alertness or other mental function, as that which loss normally accompanies prolonged periods without sleep.

The second lasting side effect exhibited in the patient also concerns his energy level, but as it is associated with his athletic ability. Without any increase in normal physical activity, such as exercise, the patient has repeatedly demonstrated the ability to lift an amount of weights which exceeds the maximum amount he was able to lift prior to the treatment by as much as two times. Still further, although lifting such amounts of weight has caused tears in various involved muscle groups, such tears have been completely healed within a couple of days.

The inventor has observed similar effects in terminally ill cancer patients using vaccines made from extracts of organisms which demonstrate similar extra-regenerative characteristics. Two symptoms which are common during the final stages of a cancer are a lack of energy and a high level of pain. When such patients have been treated with vaccines prepared from organisms exhibiting such extra-regenerative characteristics in accordance with the method of the present invention, the patients have exhibited an exceptional loss of pain and a significant increase in overall energy levels. No negative side effects of the treatment have been observed. The inventor has observed that such effects of this treatment have been highly regarded by both patient and loved ones of the patient.

Transfer of beneficial effects may be by total or partial incorporation of organism into host and may not necessitate genetic transfer to host.

### EXAMPLE 9

A ninth embodiment of the method for producing therapeutic agents according to the present invention involves another common “malady” which the inventor believes may be readily treatable via the method of the present invention: old age. An embodiment of a method for treating a patient against many of the effects of old age in accordance with the method of the present invention involves the initial step of aging in vitro, via methods which are known, various cells which have been extracted from a patient's body. After such cells have been aged, the next steps of this embodiment include: isolating any factors from the aged cells which may be associated with aging; identifying or raising organisms which are antagonistic toward the identified aging factors; preparing therapeutic agents from said antagonistic organisms in similar fashion as with earlier described embodiments of the present invention; testing the therapeutic agents; and treating the patient with the agents.

One method of identifying aging factors and raising organisms which are antagonistic toward these factors involves the use of at least a different-specie host. According to this method, a first different-specie host is inoculated with patient cells which have been aged in vitro. Once antibodies against the aged cells are raised in the first different-specie host, sera containing the antibodies is extracted via means which are known. In order to “wash” any anti-patient-specie factor from said sera, a second different-specie host of the same specie as the first different-specie host is inoculated with normal, non-aged cells from the patient. This will raise anti-patient-specie antibodies in the second different-specie host, which can then be extracted and applied in vitro to the sera extracted from the first different-specie host, for precipitating the anti-patient-specie factor out of the first host sera, and thereby leaving anti-age-factor antibodies in the sera for use in preparing therapeutic agents for the patient.

Another method which may be used to monitor the effectiveness of such anti-age-factor therapy in accordance with the present invention involves use of the Haeflic limit, which limit is the maximum number of times a cell may multiply or divide. According to this method, cells from various components of the patient's body are first aged and tested in vitro via means which are known, in order both to determine the number of cell divisions remaining in each cell, and to determine a base mean time-interval between each cell division. As the patient undergoes treatment in accordance with the present invention, which treatment is directed at overcoming aging factors that may appear in the patient's body, such Haeflic limit determination tests are subsequently in repeated intervals for identifying any change in either the number of cell divisions of particular cell types, or in the mean time-interval between such cell divisions. Such Haeflic limit tests will indicate the effectiveness of the anti-age-factor therapy.

### EXAMPLE 10

A tenth embodiment of the method of the present invention involves the creation and therapeutic use of vaccines. A vaccine is typically composed of the same or similar material as a disease, such as a fragment of the viral membrane, in order to raise an immune response to the disease. For example, popular vaccines such as tetanus, whooping cough or pertussis are made from heat-killed, or otherwise killed, extracts of tetanus, whooping cough or pertussis. Sometimes related organisms are used to create a vaccine rather than the actual disease, such as is done with the smallpox vaccine, which is made from cow-pox.

Unfortunately, it is not known how some of the targeted diseases actually work, which means that there may be great risk that vaccines which use extracts of these diseases are ultimately more harmful to the body than good. For example, current AIDS vaccines are being made from genetically engineered forms of the HIV envelope protein, in order to stimulate the immune system against the AIDS virus. Unfortunately, as previously mentioned, the immunostimulant capability of the HIV virus is highly specific, targeting only T-cells, the very cells which the HIV virus uses to further infect the body. Although the T-cell count in an AIDS patient might be temporarily increased when such vaccines are used therapeutically, the end result is not, therefore, considered by many to be therapeutic.

It is questionable, also, whether such vaccines are useful as a preventative. With HIV, the inherent risk associated with using any part of the HIV virus as a vaccine against the disease, is especially great, since there is yet a universally accepted model which explains how so small amount of a virus can so inhibit a host's immune system. To challenge the immune system of individuals who have not yet been exposed to such a deadly agent, by inoculating them with fragments of such agent, is believed by many to be quite foolish.

Indeed, because of the highly resistant nature of the HIV virus, to vaccinate an individual with HIV fragments might lead to the growth of multiple HIV mutations which will each have to be targeted for treatment, resulting in a division of effort in an already deficient immune system. Furthermore, there is a growing number of both medical practitioners and laymen who believe that such vaccine exposure to any pathogen is not good for the immune system.

A situation similar to AIDS exists with the use of current vaccines for cancer. Like AIDS, the immunostimulant activity of cancer is generally highly specific, activating only a portion of the host immune system which can be used to the benefit of the disease. The portion of the immune system which is generally stimulated by cancer comprises antibodies that are used by the disease to coat the diseased cells, for preventing the diseased cells from being visible to the immune system as foreign matter. Therefore, to stimulate the further production of such coating or blocking antibodies, is to promote further proliferation of the cancer, and thereby increasing the load of diseased tissue in the body, which may lead ultimately to threshold inhibition of the immune system. Current research work with cancer involves attempts to extract out from diseased tissue certain proteins and compounds which are capable of inducing a strong immunological response. Unfortunately, by the time the cancer is denatured into extracts which are immunologically active, such extracts bear little resemblance to the diseased tissue in the body which is inherently immunologically inactive. Therefore, the immune response that is ultimately raised by vaccines of such extracts will logically attack the vaccine, but may not associate the cancer with such vaccines. However, even should the cancer be slightly recognized and attacked by the immune system, such attack is likely to be sufficiently minimal so as to result in the cancer mutating into more resistant strains, rather than being inhibited significantly.

With the method of the present invention, however, a person's immune system can be effectively prepared for dealing with a potential biological threat by inoculation with vaccines which are not comprised of portions of the threatening target disease. Such vaccines are rather comprised of disease-associated organisms, extracts or modifications thereof, which have been raised against the target disease. For example, it has been suggested that the HIV virus blocks CD-4 receptor sites on white blood cells, and therefore, that treatments which block these sites for preventing the HIV virus from attaching to them might have some efficacy. However, again, we do not know what beneficial function such CD-4 site blocking activity might inhibit. Via the method of the instant invention, a Nemesis organism might be isolated for accomplishing such blocking by distorting the shape of the CD-4 receptors, rather than by blocking them completely. Such distortion could inhibit the attachment of the HIV virus without completely blocking the receptor sites. We could also place CD-4 receptor decoys into the patient's blood stream by fragmenting a multitude of CD-4 receptors from the cell membrane of a plurality of white blood cells and inoculating the patient with said CD-4 receptors. As each cell of the HIV virus attaches to such a CD-4 receptor decoy, it becomes enabled from attaching to a live white blood cell. A further method of preventing the attachment of HIV virus to white blood cells by using decoy cellular material includes the step of enucleation, in which the nucleus of a plurality white blood cells is spun out of each cell via centrifugal in vitro techniques which are known. The resultant nucleus-free white blood cells are then inoculated into the patient's blood stream as decoys for the HIV virus. Because there is no nuclear material in such white blood cells, when cells of the HIV virus attach to their CD-4 receptors, the HIV will be inhibited from causing the white blood cells to transform into diseased tissue. One may also insert into such nucleus-free white blood cells, material which is antagonistic to the HIV virus, such as a chemical or an anti-body, for destroying the HIV cells which attach to the cell decoy. The nuclear-free white blood cells may also be tagged with disease-associated organisms or extracts thereof for making such cells visible to the immune system.

One version of such method involves placing bacterial antigens inside the nuclear free white blood cell, so that when a subsequently attached HIV cell begins to destroy the white blood cell, the bacterial antigens mark each of the CD-4 receptor sites on a surface oppositely disposed to the HIV cell, for the host immune system to identify and destroy. Similar tagging techniques can be used with the decoy HIV membrane fragments mentioned earlier. The use of Nemesis and antagonistic organisms in vaccine therapy, in accordance with the present invention, depends on the following: 1) the Nemesis phenomenon; 2) an interference phenomenon; and 3) a regenerative rerouting or utilization of more appropriate organism response phenomena.

In accordance with the method of the present invention, causes of diseases are looked for; therapeutic mechanisms which have not been heretofore available are used; and immunological mechanisms may be regenerated, regulated and rerouted, or even created. Furthermore, in using the method of the present invention, some portions of the host body may be stimulated to perform an entirely different function than that which has heretofore been associated with said body portions. For example, some portions of the blood system which heretofore have not been associated with immunological activity may be stimulated to act immunologically; and, other cells or organs which have not been heretofore associated with the production of hormones, may be stimulated to begin such hormone production in the absence of the main body organ normally associated with such hormone production.

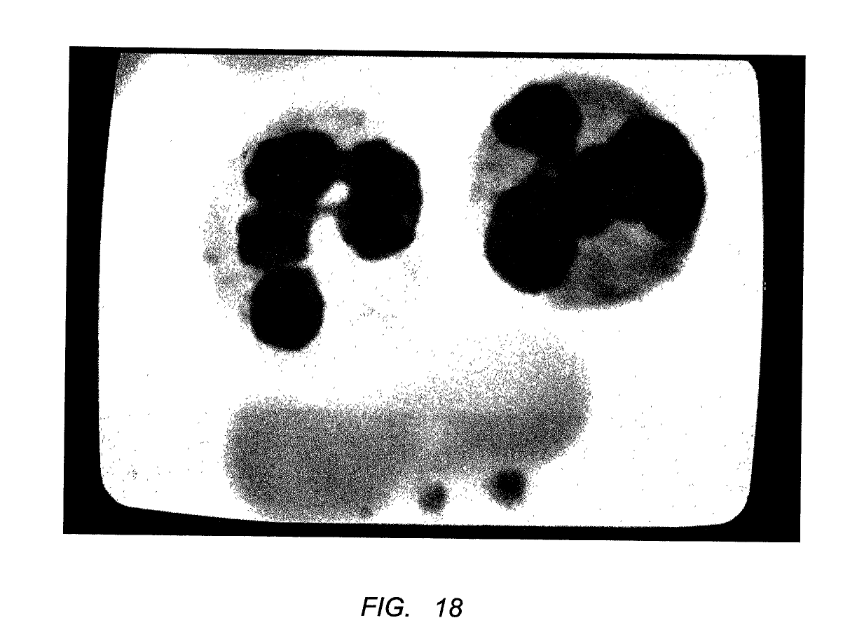

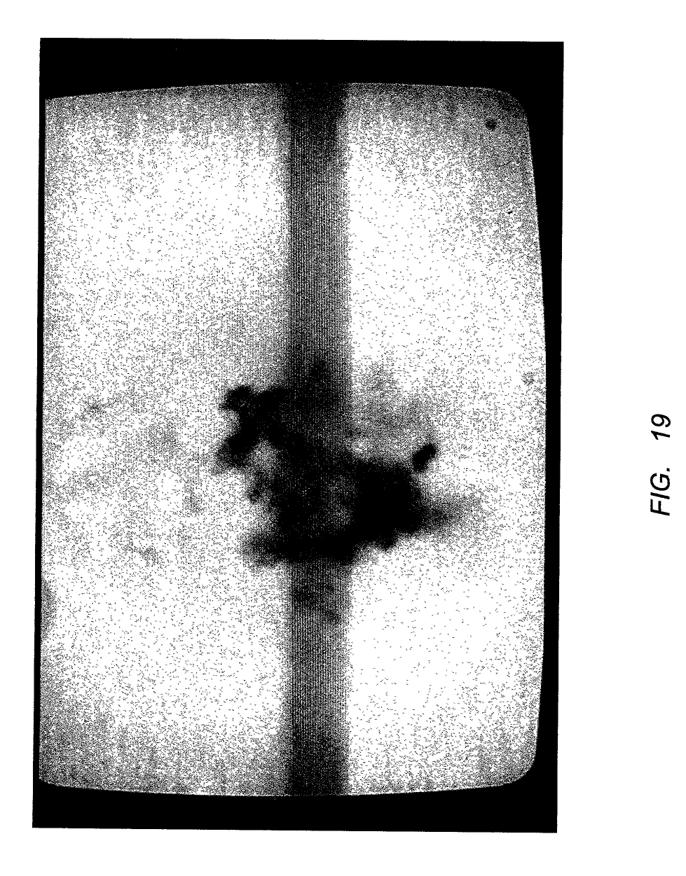

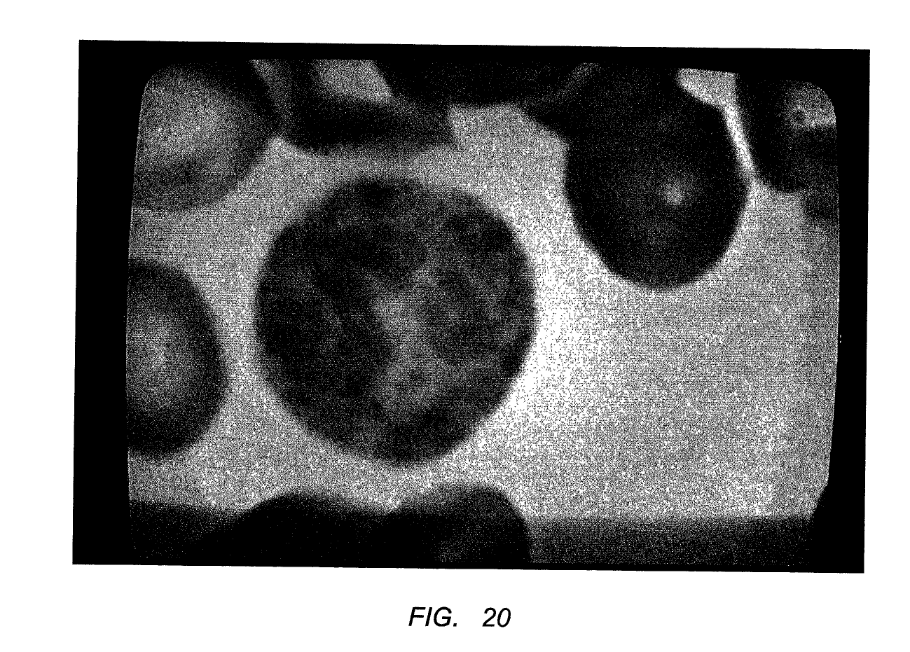

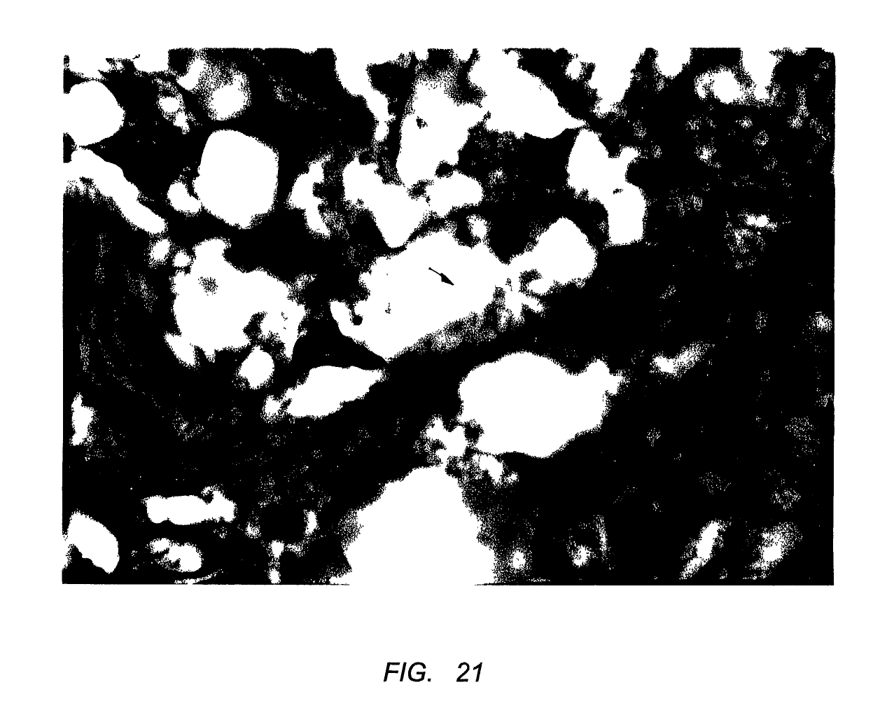

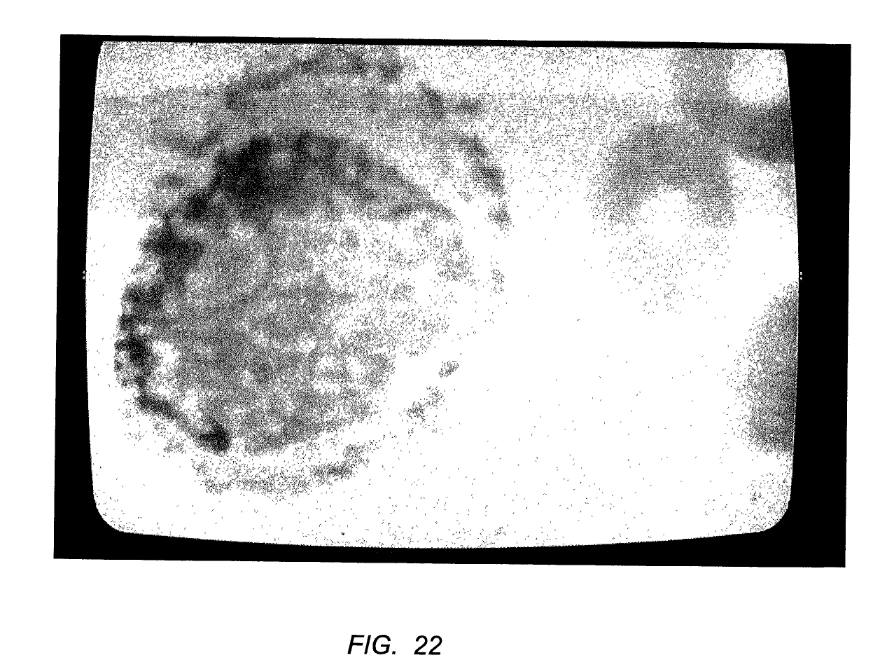

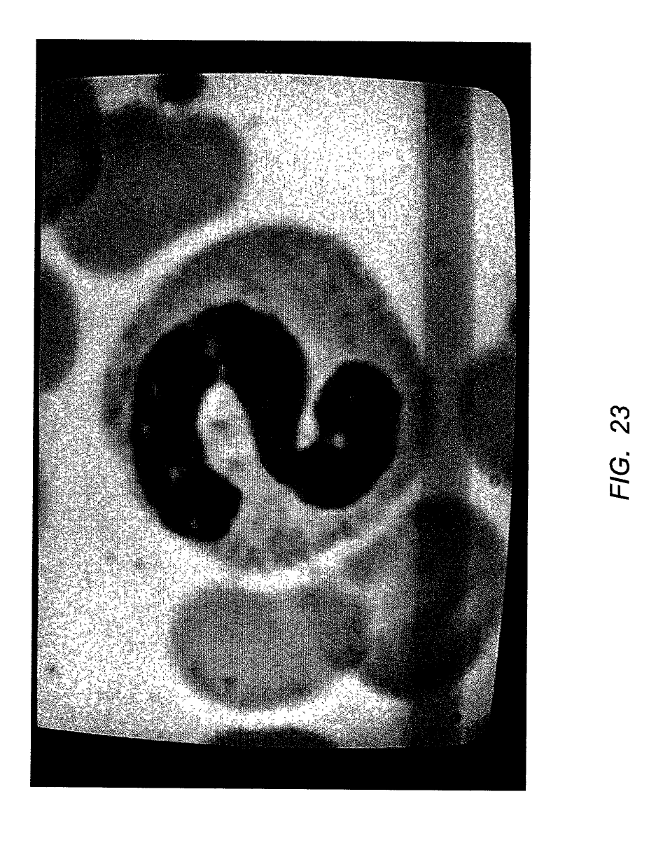

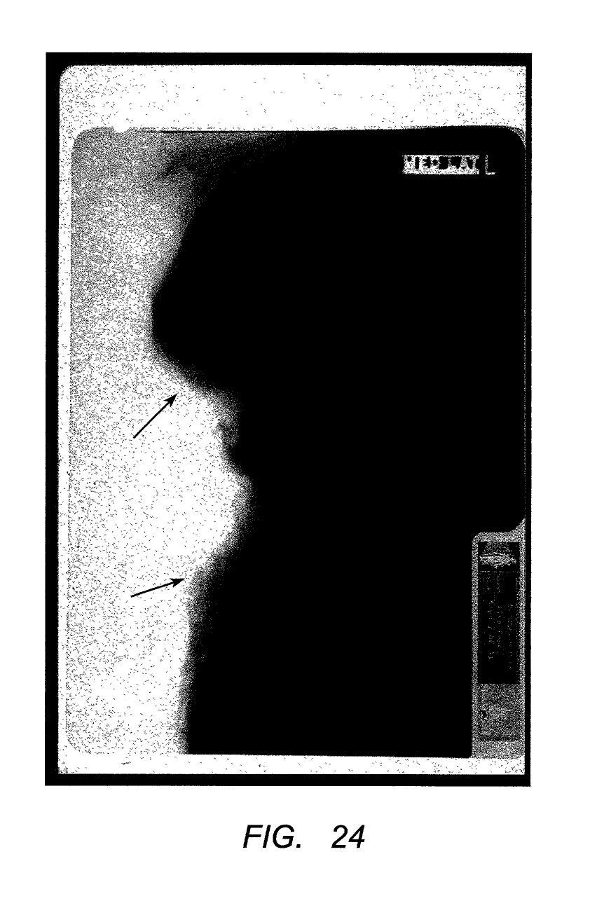

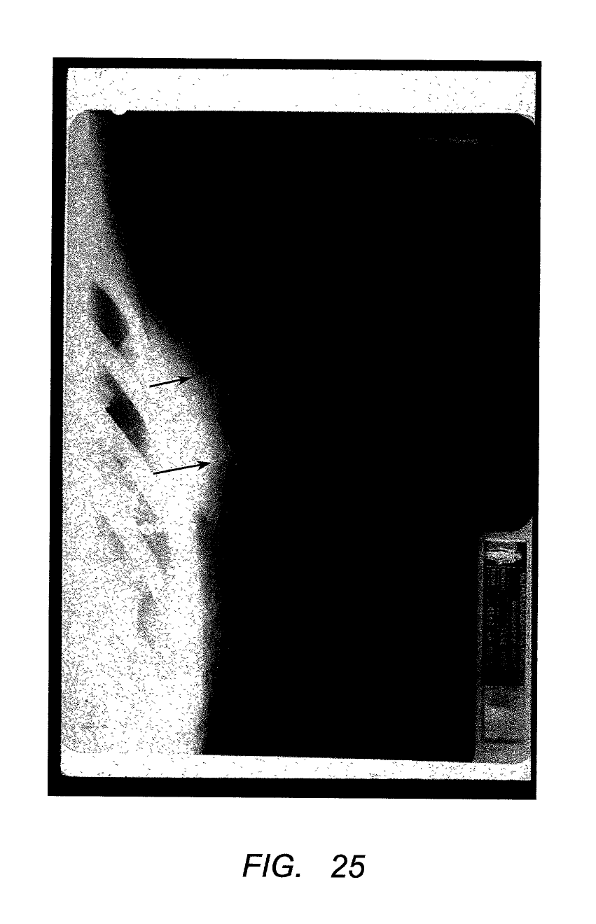

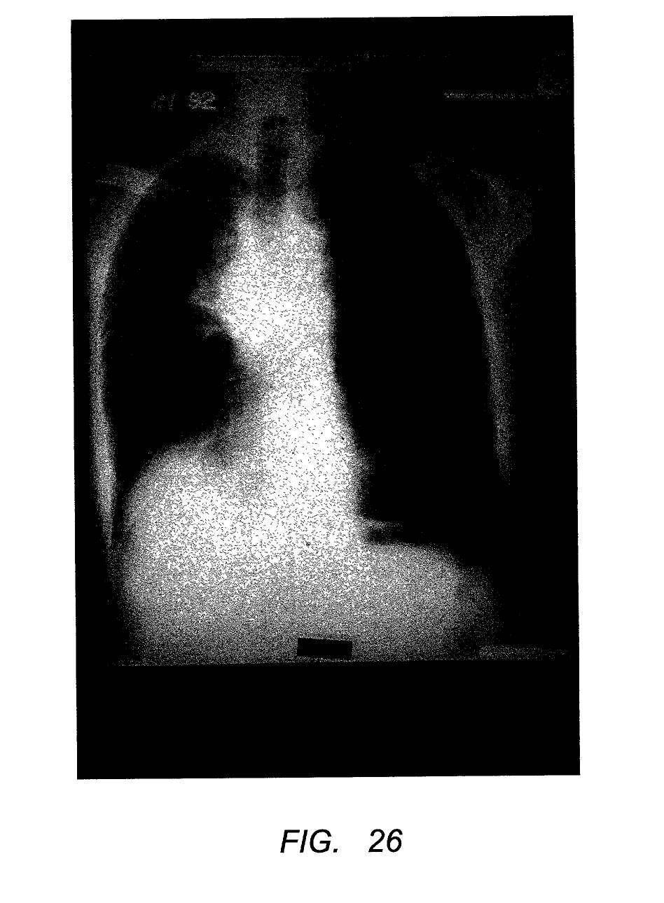

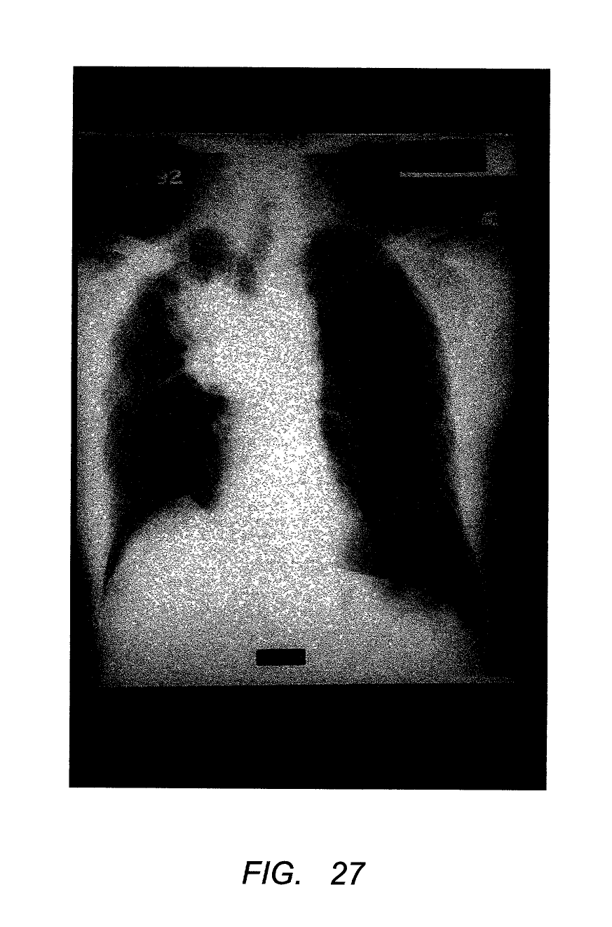

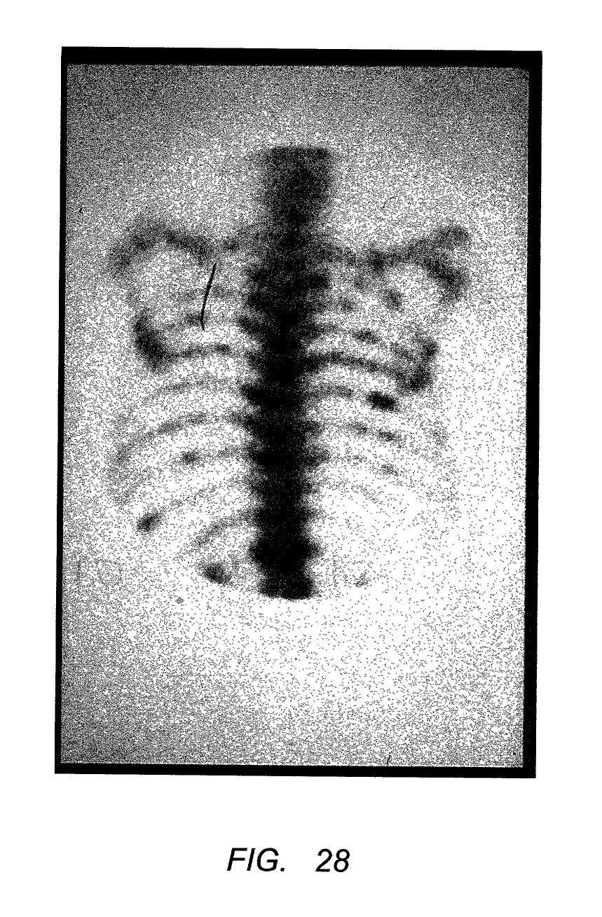

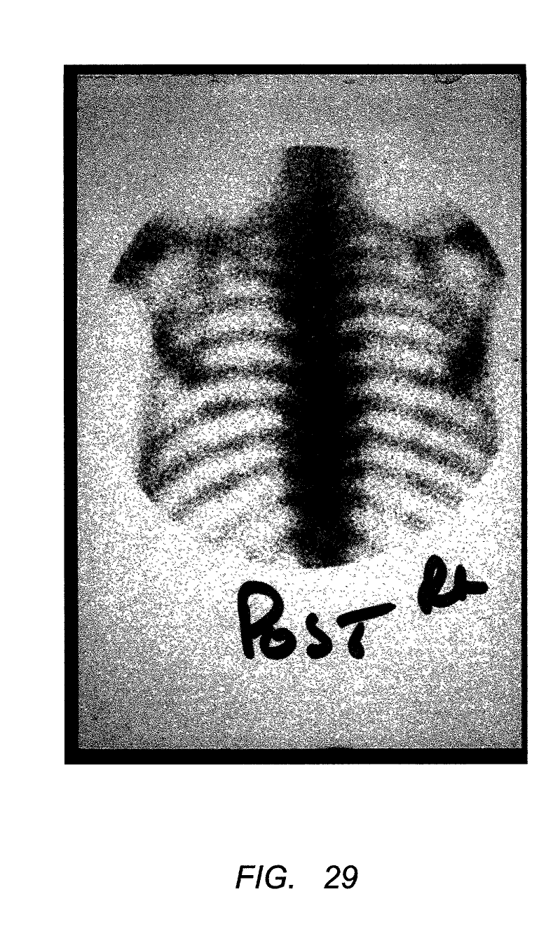

Examples of specificity and efficacy of this technology are provided by the figures. FIG. 18 illustrates 2 leukemia cells in proximity to the red blood cells and other blood constituents. When antiserum is prepared against the leukemia cells by prior art teachings, the antiserum will often contain anti-human antibodies and result in total cell lysis of both leukemia and normal cell constituents. FIG. 19 shows total lysis of leukemic and red blood cells within minutes of addition of antiserum raised against the leukemia cells. FIG. 20 shows a leukemic cell surrounded by red blood cells. FIG. 21 shows coccal organisms in a sarcoma biopsy. Antisera raised against these are used to treat the leukemia (also a sarcoma) blood in FIG. 20. FIG. 22 shows lysis of cancer cytoplasm, membrane and nucleus with no harm to surrounding red blood cells. This change occurred within minutes. FIG. 23 illustrates both precision and potential of this technology in a leukemia cell treated by antiserum raised against genetic and other fragments of associated organisms. When the antiserum is prepared against the organisms associated with leukemia or even certain other sarcomas such as those shown in FIG. 3 and the cocci shown in FIG. 20. Nuclear vacuolation can be seen where presumably abnormal cellular genetics have been removed. Cells in culture will now behave more normally. Direct extracts from nemeses and antagonistic organisms may also result in similar changes. FIG. 24 shows a large breast cancer with central ulceration as seen in the mammogram of a 72 year old female. FIG. 25 is the same patient showing dramatic reduction in mass after 2 weeks of therapy. Arrows indicate cancer margins in FIGS. 24 and 25. FIG. 26 is of a squamous cell carcinoma indicated by the arrow, stretching to the apex of the right lung. FIG. 27 shows collapse of that mass within 2 weeks of therapy. FIG. 28 is of a bone scan demonstrating prostate cancer metastases. These can be seen as the dark marks on the ribs indicated by the arrows. FIG. 29 shows drastic resolution of the rib lesions following 3 weeks of therapy.

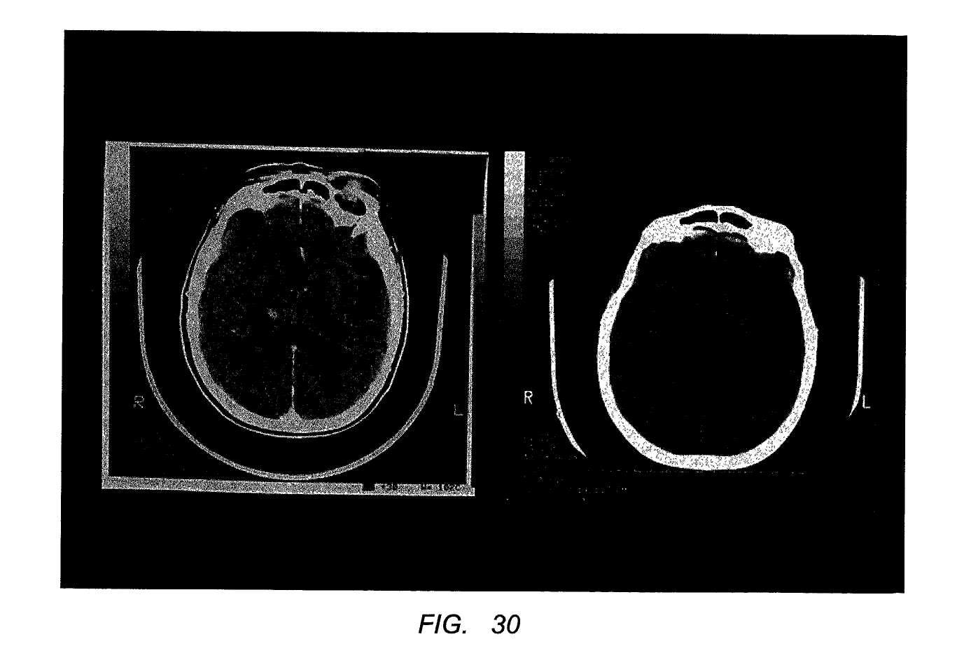

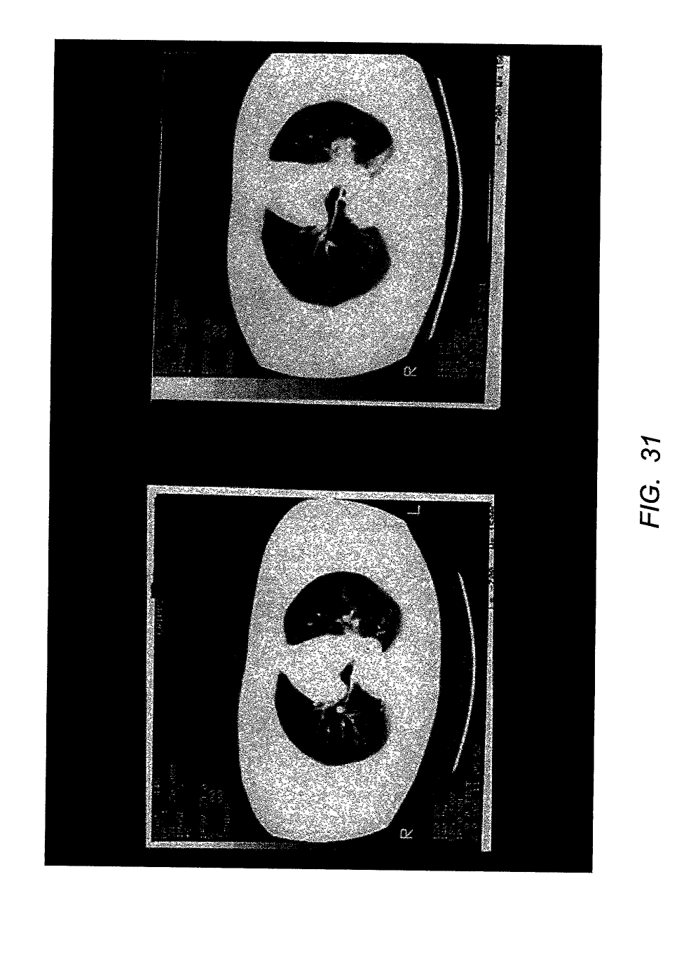

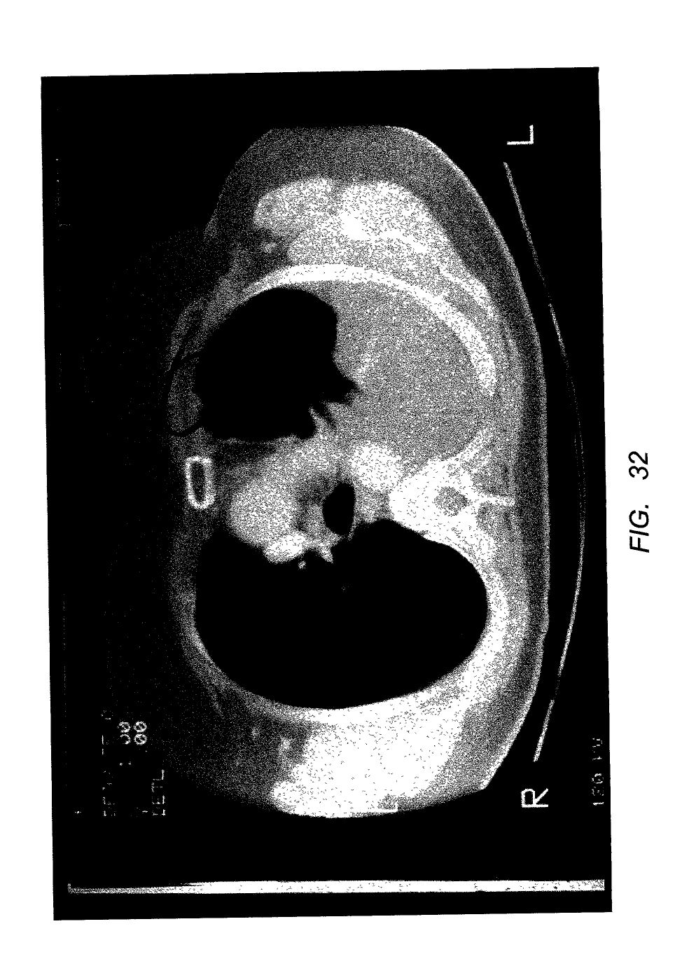

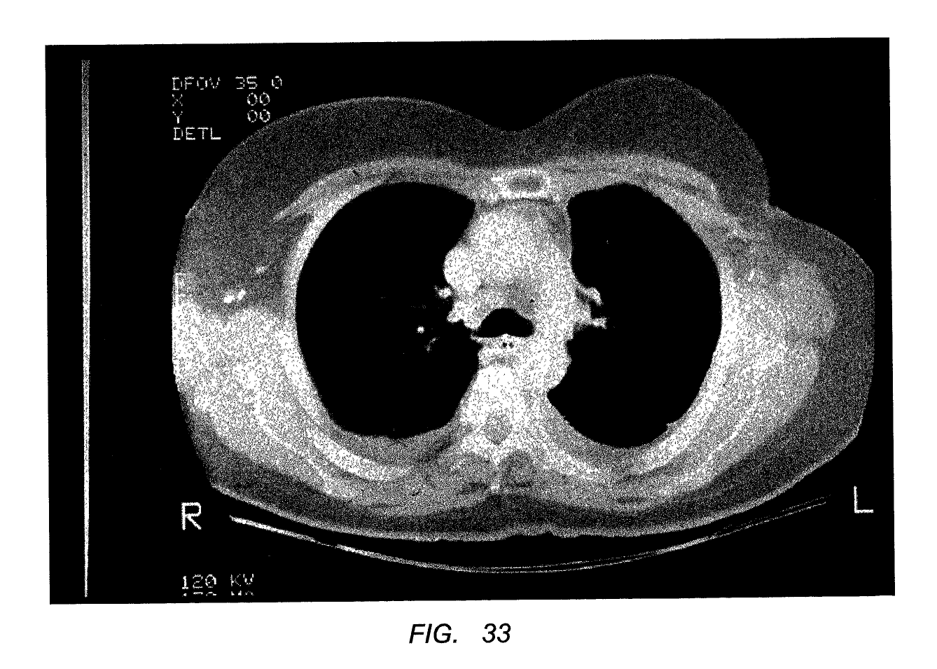

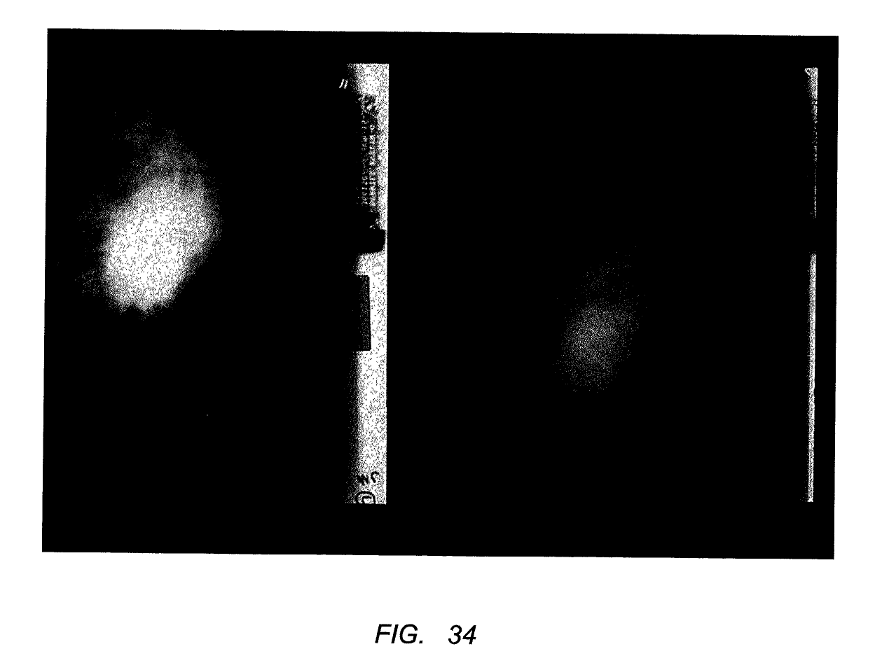

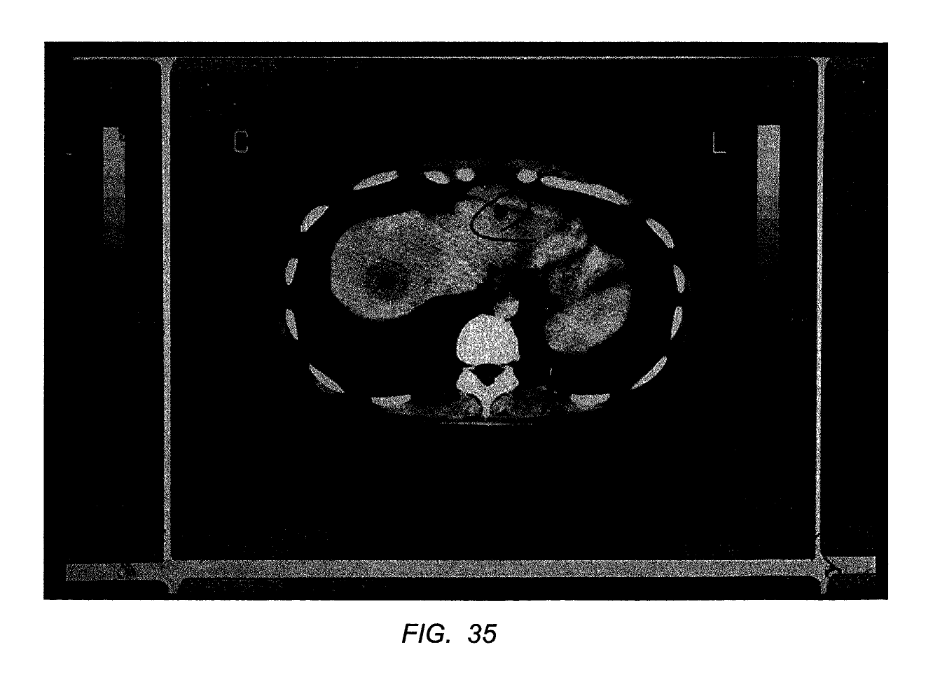

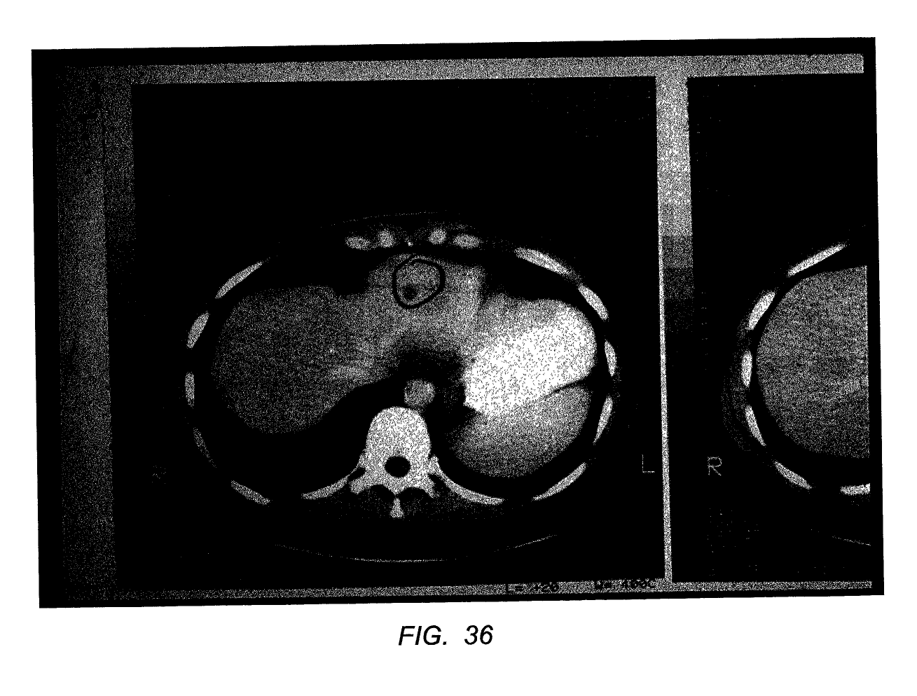

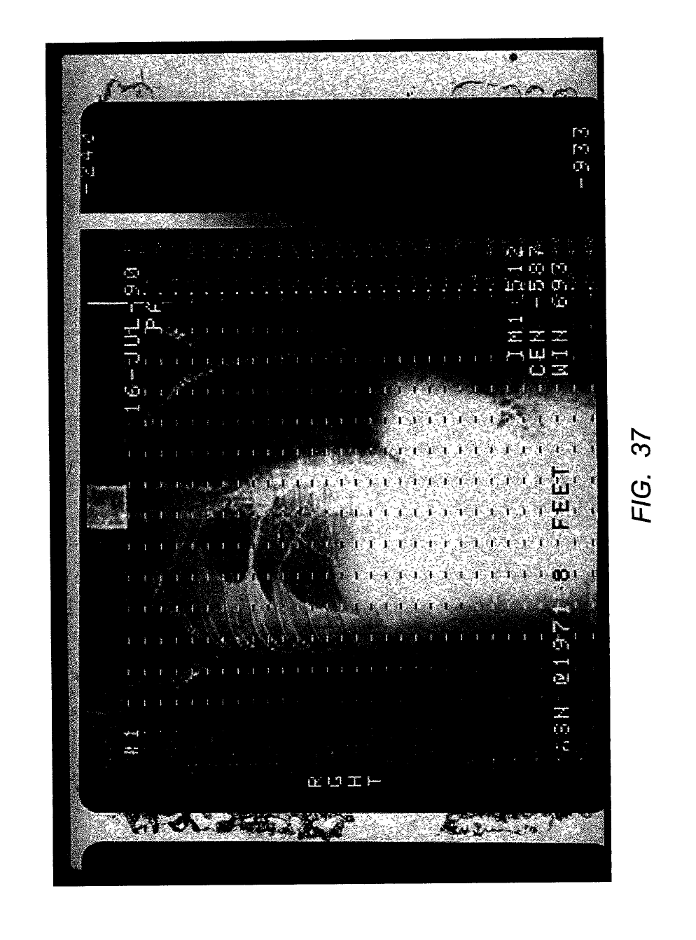

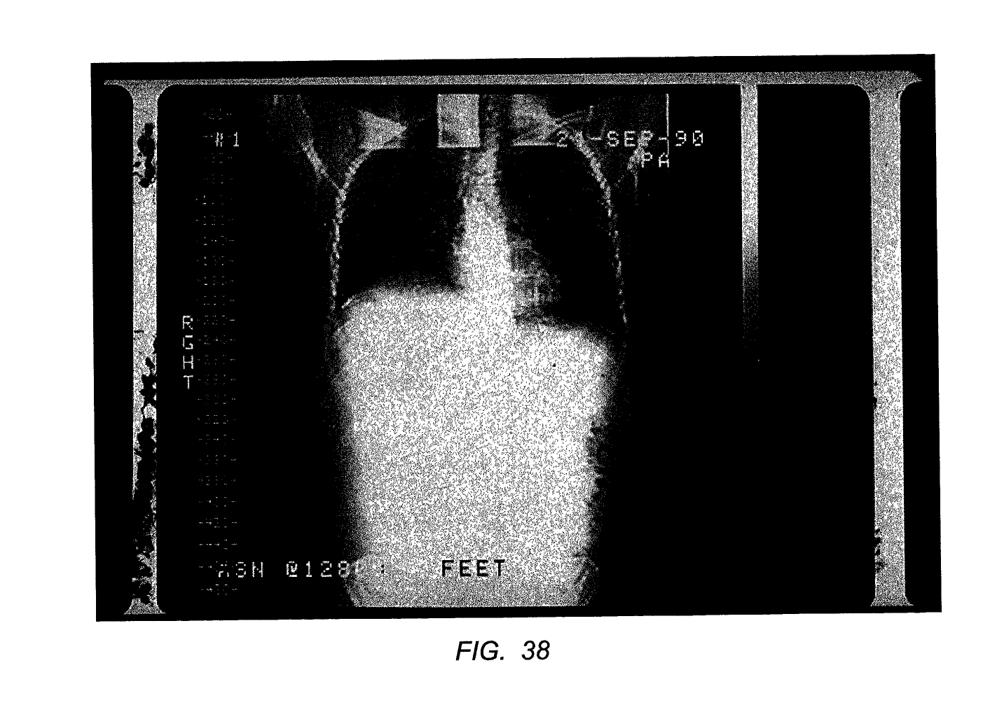

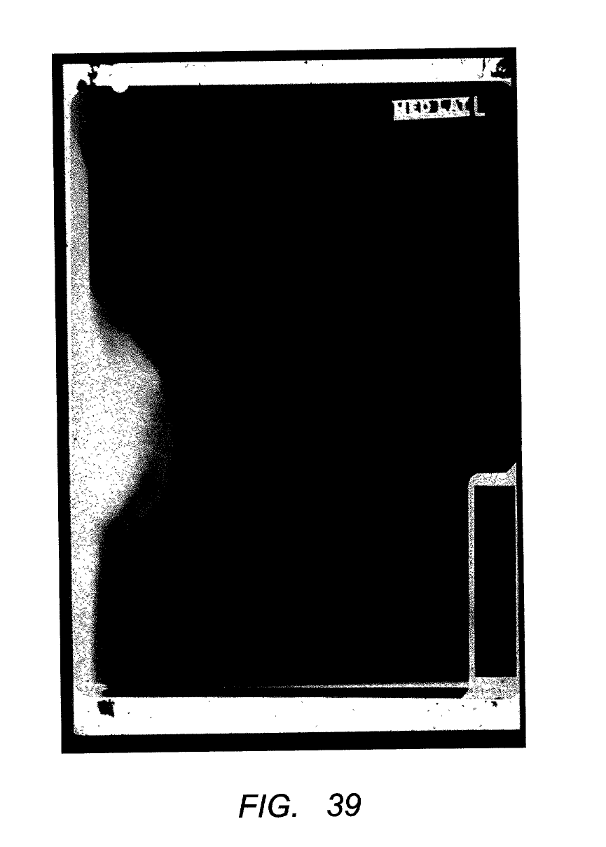

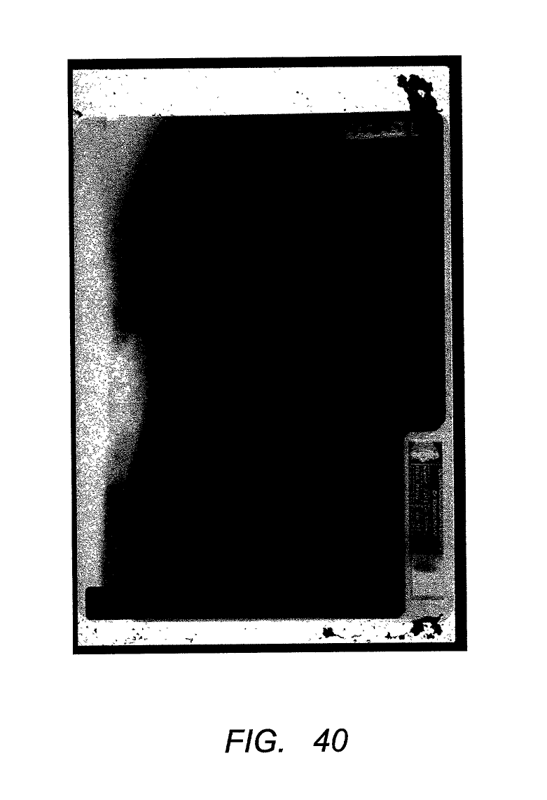

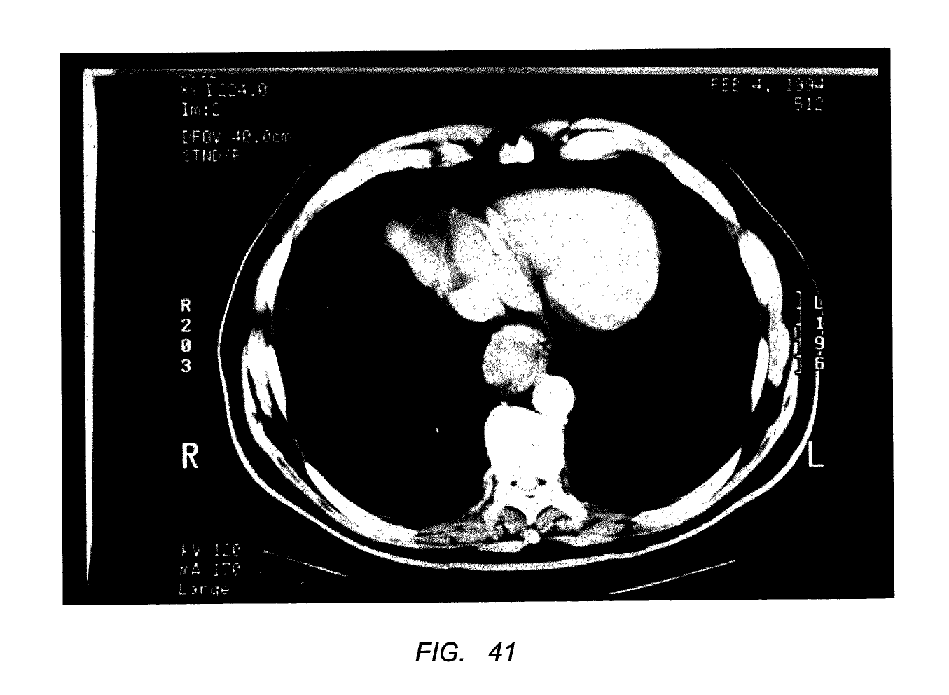

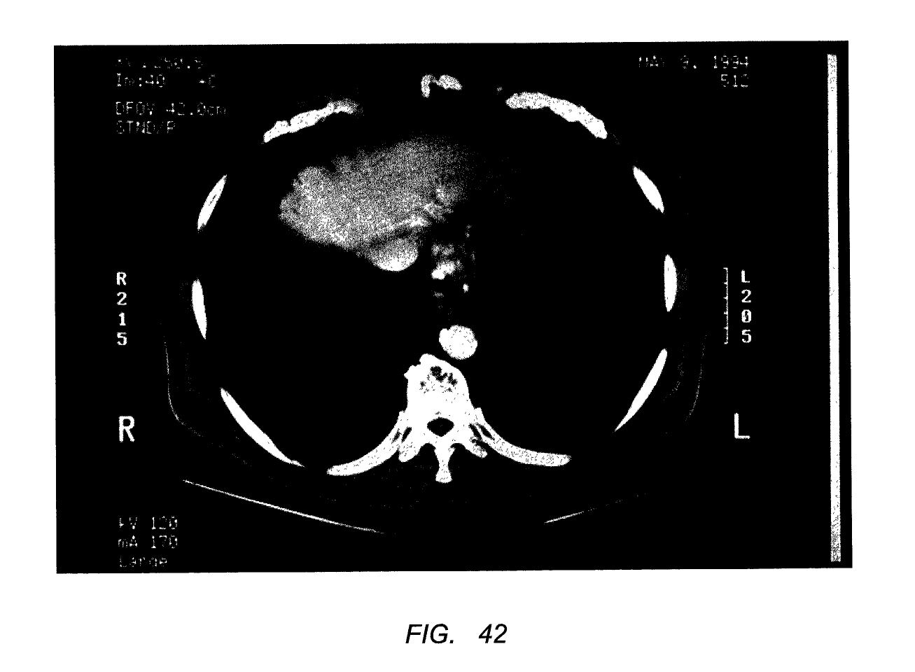

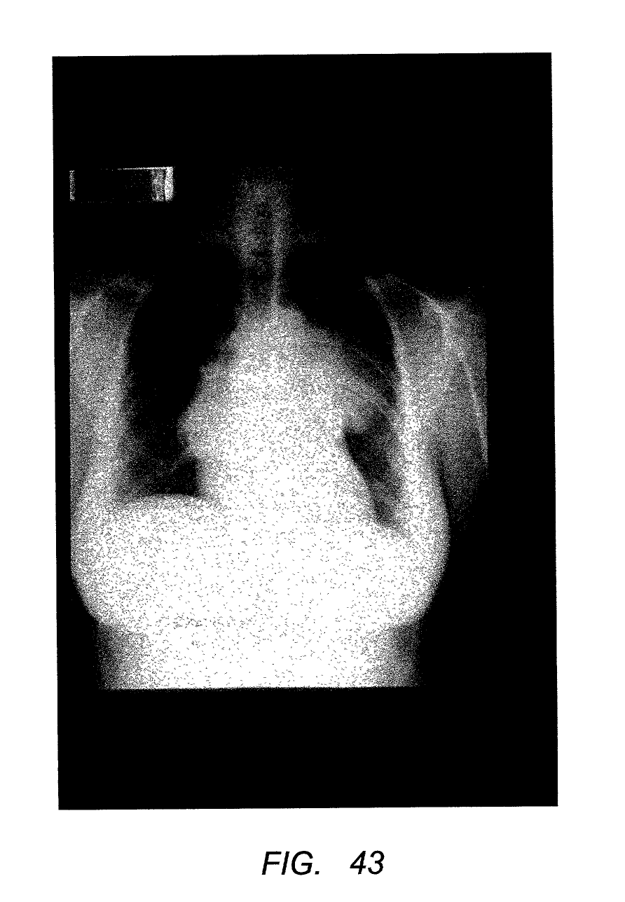

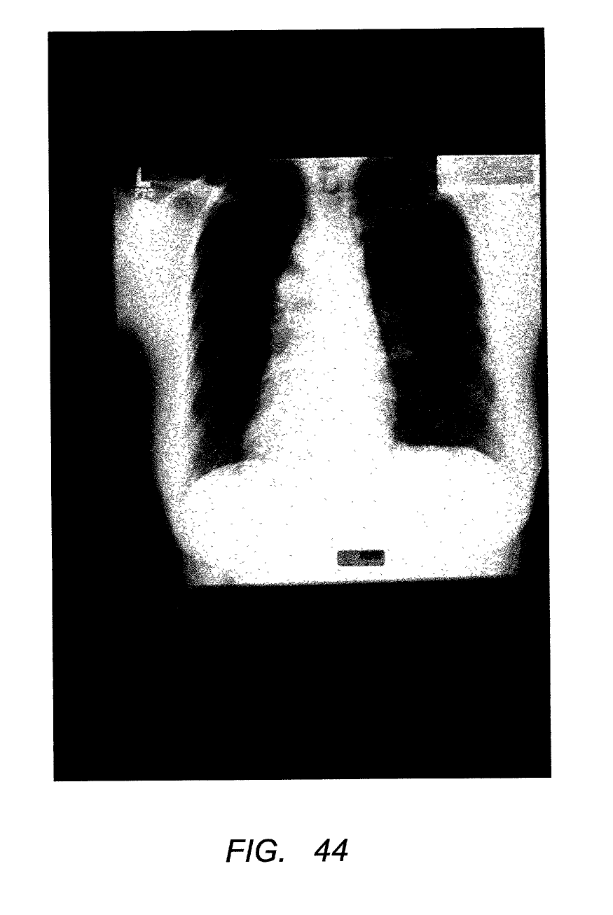

FIG. 30 demonstrates brain metastases in the right hemisphere from a small cell carcinoma [lung primary] indicated by arrows. Picture showing resolution is beside it and to the right. Total disappearance of the one lesion and shrinkage of the other occurred within 2 months of therapy. FIG. 31 demonstrates the lung primary referred to above and its mediastinal spread. Picture to the right demonstrates resolution within 2 months of therapy. FIG. 32 represents a CAT scan of breast cancer metastasized to the left lung with mass and fluid marked by the arrow. FIG. 33 demonstrates resolution within 5 weeks of therapy. FIG. 34 is of an adenocarcinoma of the breast. Mammograms show marked shrinkage within 1 month. FIG. 35 demonstrates a metastasis from breast cancer into the liver. FIG. 36 demonstrates resolution of breast cancer after 2 weeks of therapy. FIG. 37 is of a primary hepatoma perforating the right hemidiaphragm and surrounding the right lung. FIG. 38 shows cancer eliminated from the right lung field. FIG. 38 shows an adenocarcinoma of the breast in a 42 year old female as shown by mammogram. FIG. 39 shows massive shrinkage after 4 weeks of therapy. FIG. 40 shows a large mass obstructing the esophagus in a male age 60 suffering from esophageal cancer marked by the arrow. Patient is unable to swallow food or water at this stage. FIG. 41 shows that after only 4 weeks treatment the cancer shrunk massively. Patient's esophagus is patent, and he is able to eat and swallow easily. FIG. 42 illustrates giant cell lymphoma in a 32 year old female, 16 cm in diameter as measured on chest X-ray. FIG. 43 shows the same tumor after 1 week of treatment shrunk to 3 cm. The above cases are marked not only by the dramatic response time, but also by the fact that most cancers represented had already failed from conventional radiotherapy and chemotherapy. The inventor has given a non-limiting description of several embodiments of the present invention, to which many changes may be made without deviating from the spirit of the inherent inventive concept. While this invention has been described with reference to such illustrative embodiments, this description is not intended to be construed in a limiting sense. Various modifications and combinations of the various embodiments as well as other embodiments of this invention will be apparent to a person skilled in the art upon reference to this description. It is therefore contemplated that the appended claims cover any such modifications and/or embodiments that fall within the true scope of the present invention.

I claim:

1. A method for identifying at least one therapeutic agent for the treatment against a targeted disease comprising:
developing a database of biological, chemical, and physical agents and organisms, without regard to any known therapeutic value, which database includes activity levels of said agents and organisms toward diseases;
selecting a targeted disease;
determining a prescribed activity level for said targeted disease;
conducting a spectrum search of said database to determine all agents and organisms meeting said prescribed activity level for said targeted disease; and
choosing at least one of said agents and organisms which best satisfies a predetermined treatment strategy for use as a therapeutic agent.

2. The method of claim 1, wherein said selected at least one agent and organism is itself a disease-causing agent or organism.

3. The method of claim 1, further comprising using the identified therapeutic agent as a preventative agent, a vaccine, a diagnostic agent, a prognostic agent, a monitoring agent or an energizing agent.

4. The method of claim 1, wherein said spectrum search includes an in vivo search.

5. The method of claim 1, wherein said spectrum search includes an in vitro search.

6. The method of claim 1, wherein said spectrum search includes a search among organisms found within a patient with the targeted disease.

7. The method of claim 1, wherein said spectrum search includes a search among organisms found within a host organism substantially similar to a patient with a targeted disease.

8. The method of claim 7, wherein said host organism has the targeted disease but said targeted disease is in remission.

9. The method of claim 7, wherein said host organism is a different specie than the patient.

10. The method of claim 7 further comprising:
raising in a second host organism at least one antibody to normal cells of a patient with the targeted disease; and
applying said antibody in vitro to the targeted disease for precipitating any anti-patient-specie factor out of the antibody raised in the first host organism.

11. The method of claim 1 further comprising infecting said chosen agent or organism with at least one phage having a microbial affinity toward the targeted disease whereby the targeted disease is subjected to infection by the phage.

12. The method of claim 1, wherein said spectrum search includes a search among agents precipitated by an immunological response of a patient with the targeted disease.

13. The method of claim 1 further comprising testing the affinity of at least one agent or organism toward the targeted disease whereby said targeted disease may be tagged to be more visible to the immune system of a patient with the targeted disease.

14. The method of claim 13 wherein said testing includes testing radioisotopes.

15. The method of claim 1 further comprising administering the identified therapeutic agent orally, rectally, by injection directly into diseased tissue, intradermally, intraurethrally, intravesically, by inhalation, intradurally, by catheter, intravenously, intramuscularly, subcutaneously, extrademally, as a food or as a dietary supplement.

16. A method for making a therapeutic complex for use in treating a patient having a targeted disease or condition comprising selecting a disease-associated agent from the group consisting of a causative, synergistic, neutral, infective.
